(19) United States
(12) Patent Application Publication
(10) Pub. No.: US 2025/0134130 A1
(43) Pub. Date: May 1, 2025

(54) RECOMBINANT PROTEIN RECOVERY METHODS AND USES THEREOF

(71) Applicant: Clara Foods Co., Daly City, CA (US)

(72) Inventor: Aniket KALE, Pleasanton, CA (US)

(73) Assignee: Clara Foods Co., Daly City, CA (US)

(21) Appl. No.: 18/907,140

(22) Filed: Oct. 4, 2024

Related U.S. Application Data
(60) Provisional application No. 63/587,977, filed on Oct. 4, 2023.

(51) Int. Cl. A23J 1/00; A21D 2/26; A23J 3/22; B01D 21/26; B01D 61/14; C07K 14/77; C12N 15/80

(52) U.S. Cl. CPC A23J 1/008; A21D 2/267; A23J 3/227; B01D 21/262; B01D 61/145; B01D 61/147; B01D 61/149; C07K 14/77; C12N 15/80

(57) ABSTRACT

The present disclosure relates to methods for increasing recovery and purity of a secreted protein of interest, recovered proteins from the methods, and uses of the proteins.

### 1. BACKGROUND

The recombinant proteins can be produced by precision fermentation by expressing in multiple host systems such as bacteria, yeast, and fungi. Fungal systems have an inherent issue in that they express both a protein of interest and native fungal proteins. Thus, when using fungal expression systems, additional, complicated, time-consuming, and expensive steps are needed for recovering and purifying proteins of interest. There remains an unmet need for simplified recovery and purification of proteins of interest from fungal expression systems.

### 2. SEQUENCE LISTING

The instant application contains a Sequence Listing which has been submitted via Patent Center and is hereby incorporated by reference in its entirety. Said .xml copy, created on Sep. 26, 2024, is named 41522-60654_US.xml and is 98,877 bytes in size.

### 3. SUMMARY

The instant application discloses methods and compositions for recovery and/or purifying a secreted protein of interest. Methods of compositions of exemplary food items containing the recovered or purified secreted protein of interest are also disclosed.

In one aspect, disclosed is a method for increasing recovery and purity of a secreted protein of interest, the method comprising steps of: obtaining recombinant fungal cells capable of expressing a secreted protein of interest; culturing the recombinant fungal cells under conditions that promote expression and secretion of the recombinant protein of interest into a culturing medium; introducing ammonium sulfate to the culturing medium to achieve an ammonium sulfate concentration above 200 g/l, thereby precipitating the secreted protein of interest; recovering the precipitated protein of interest; and diafiltering and/or ultrafiltering the precipitated secreted protein of interest.

The method may comprise a step of centrifuging the culturing medium to remove the recombinant fungal cells and other cellular components prior to introducing ammonium sulfate. In some embodiments, the centrifuged culturing medium is not microfiltered prior to introducing ammonium sulfate.

In accordance with any of the embodiments, the method comprises a step of microfiltering (MF) the diafiltered (DF) and/or ultrafiltered (UF) protein of interest. The microfiltering May comprise a filter capable of capturing fungal cells and other cellular components. In some embodiments, the filter is a 0.1 μm, 0.2 μm, 0.3 μm filter, or larger.

In accordance with any of the embodiments, the method comprises adding an agent to maintain the pH at about 4.5 to about 6.5 after centrifuging the culturing medium. In some embodiments, the centrifuged culturing medium is maintained at about pH 6.0. In some embodiments, the agent is an acid or a base. In some embodiments, the acid is phosphoric acid, e.g., 85% v/v phosphoric acid. In some embodiments, the base is sodium hydroxide.

In accordance with any of the embodiments, adding the agent occurs before introducing the ammonium sulfate. Adding the agent may occur after introducing the ammonium sulfate, or adding the agent is contemporaneous with introducing the ammonium sulfate.

In accordance with any of the embodiments, the method comprises recovering the precipitated protein of interest in a medium having a pH of about 4.5-6.5. The precipitated protein of interest can be recovered at a pH of at least 4.5, at least 5.0, at least 5.5, or at least 6.0. In some embodiments, the precipitated protein of interest is recovered at a pH of about 4.5, about 5.5, or about 6.0. In some embodiments, the precipitated protein of interest is recovered at a pH of about 6.0.

In accordance with any of the embodiments, the method comprises a step of drying the further microfiltered protein of interest, thereby obtaining a dried protein product. The method may further comprise a step of solubilizing the precipitated secreted protein of interest with water, e.g., DI water, to obtain a solubilized protein of interest prior to ultrafiltering the precipitated secreted protein of interest.

In accordance with any of the embodiments, the ammonium sulfate concentration is above 200 g/l, the ammonium sulfate concentration for precipitating the protein of interest is above 300 g/l, or the ammonium sulfate concentration is above 400 g/l. In some embodiments, the ammonium sulfate concentration is about 200 g/l, the ammonium sulfate concentration is about 300 g/l, or the ammonium sulfate concentration at is about 400 g/l. In some embodiments, the amount of recovered protein of interest is greater than the recovery that is obtained from a method that does not comprise adding ammonium sulfate to the culturing medium to achieve an ammonium sulfate concentration above 200 g/l.

In accordance to any of the embodiments, recovery of the protein of interest is at least about 40% w/w, is at least about 45% w/w, is at least about 50% w/w, is at least about 55% w/w, is at least about 60% w/w for the final dried product, is at least about 65% w/w for the final dried product, is at least about 70% w/w for the final dried product, is at least about 75% w/w for the final dried product, is at least about 80% w/w for the final dried product, is at least about 85% w/w for the final dried product, or is at least about 90% w/w for the final dried product, wherein the recovery is the weight of the protein of interest recovered in the final product relative to the weight of the protein of interest prior to introducing ammonium sulfate.

In accordance to any of the embodiments, recovery of the protein of interest is at least about 40% w/w, is at least about 45% w/w, is at least about 50% w/w, is at least about 55% w/w, is at least about 60% w/w, is at least about 65% w/w, is at least about 70% w/w, is at least about 75% w/w, is at least about 80% w/w, is at least about 85% w/w, is at least about 90% w/w, for the final dried product, wherein the recovery is the weight of the protein of interest recovered in the final product relative to the sum of weight of the protein of recovered and the weight of the protein remaining in the supernatant following introducing ammonium sulfate.

In some embodiments, the method does not comprise use of a purification resin and/or a purification column. In some embodiments, the method does not consist of use of a purification resin and/or a purification column.

In accordance with any of the embodiments, the fungal cells are of the species selected from Agaricus bisporus; Agaricus spp.; Aspergillus awamori; Aspergillus fumigatus; Aspergillus nidulans; Aspergillus niger; Aspergillus oryzae; Aspergillus oryzae; Aspergillus spp.; Colletotrichum gloeosporiodes; Colletotrichum spp.; Endothia parasitica; Endothia spp.; Fusarium graminearum; Fusarium solani; Fusarium spp.; Komagatella pastoris; Komagatella phaffi; Mucor miehei; Mucor pusillus; Mucor spp.; Myceliophthora spp.; Myceliophthora thermophila; Neurospora crassa; Neurospora spp.; Penicillium (Talaromyces) emersonii; Penicillium camemberti; Penicillium canescens; Penicillium chrysogenum; Penicillium funiculosum; Penicillium purpurogenum; Penicillium roqueforti; Penicillium spp.; Pichia angusta; Pichi pastoris; Pichia pastoris; Pichia Pastoris “MutS” strain (Graz University of Technology (CBS7435MutS) or Biogrammatics (BG11)); Pichia spp.; Pleurotus ostreatus; Pleurotus spp.; Rhizomucor miehei; Rhizomucor pusillus; Rhizomucor spp.; Rhizopus arrhizus; Rhizopus oligosporus; Rhizopus oryzae; Rhizopus spp.; Trichoderma altroviride; Trichoderma reesei; Trichoderma spp.; Trichoderma vireus; Yarrowia lipolytica; and Yarrowia spp.

In some embodiments, the fungal cells are Aspergillus cells. In some embodiments, the Aspergillus cells are of the species Aspergillus spp., Aspergillus awamori, Aspergillus fumigatus, Aspergillus nidulans, Aspergillus niger, or Aspergillus oryzae. In some embodiments, the Aspergillus cells are Aspergillus niger cells.

In accordance with any of the embodiments, the protein of interest is a food protein. The food protein can be used as nutritional, dietary, digestive, supplements, such as in food products and feed products. The food protein can be a plant protein or an animal protein.

In some embodiments, the animal protein is an egg white protein. The egg white protein can be selected from ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

In some embodiments, the egg white protein is an ovalbumin (OVA) that comprises the amino acid sequence of a chicken OVA, a goose OVA, a quail OVA, an ostrich OVA, or a duck OVA.

In some embodiments, the egg white protein has at least 70%, 75%, 80%, 85%, 90%, 95%, 96%, 97%, 98%, or 99% sequence identity to one of SEQ ID NO: 1 to SEQ ID NO: 74.

In some embodiments, the food protein is a recovered recombinant OVA (rOVA). A glycosylation pattern of the rOVA can be devoid of N-linked galactose units. The amino acid sequence of the rOVA may lack an N-terminal methionine. In some embodiments, the rOVA is glycosylated. In some embodiments, the rOVA is non-glycosylated.

In accordance with any of the embodiments, the recovered protein of interest is suitable for use in a food product. The food product may have an additional characteristic equivalent to or better than a similar baked item made with a natural egg white or a natural whole egg, wherein the characteristic is selected from the group consisting of foam capacity, foam stability, hardness, chewiness, guminess, and springineness. The food product can be selected from the group consisting of a cake, pound cake, cookie, bagel, biscuit, bread, muffin, cupcake, scone, pancake, macaroon, choux pastry, and soufflé. In some embodiments, the food product is a burger patty. In some embodiments, the food product is a cake or a pound cake. In some embodiments, the food product is a baked food product.

Another aspect of the present disclosure includes a powdered composition comprising the recovered protein of interest in accordance with any of the embodiments.

Another aspect of the present disclosure includes a liquid composition comprising the powdered composition in accordance with any of the embodiments in accordance with any of the embodiments a solvent suitable for animal or human consumption.

In one aspect, disclosed is a method for increasing recovery and purity of a secreted protein of interest, the method comprising steps of:
obtaining recombinant fungal cells capable of expressing a secreted protein of interest;
   culturing the recombinant fungal cells under conditions that promote expression and secretion of the recombinant protein of interest into a culturing medium;
centrifuging the culturing medium to remove the recombinant fungal cells and other cellular components;
introducing ammonium sulfate to the culturing medium to achieve an ammonium sulfate concentration above 200 g/l, thereby precipitating the secreted protein of interest, wherein the centrifuged culturing medium is not microfiltered prior to introducing the ammonium sulfate; recovering the precipitated protein of interest;
solubilizing the precipitated secreted protein of interest with water to obtain a solubilized protein of interest;
diafiltering and/or ultrafiltering the solubilized protein of interest; microfiltering the diafiltered and/or ultrafiltered protein of interested; and
drying the further microfiltered protein of interest, thereby obtaining a dried protein product.

In one aspect, disclosed is a baked food item comprising a recovered and purified secreted protein of interest in accordance with any of the embodiments. In some embodiments, the baked food item has an additional characteristic equivalent to or better than a similar baked item made with a natural egg white or a natural whole egg, wherein the characteristic is selected from the group consisting of foam capacity, foam stability, hardness, chewiness, guminess, and springineness. In some embodiments, the baked food item is a pound cake.

Disclosed is also a non-meat food item comprising a recovered and purified secreted protein of interest in accordance with any of the embodiments. In some embodiments, the non-meat food item has an additional characteristic equivalent to or better than a similar non-meat item made with a natural egg white or a natural whole egg, wherein the characteristic is selected from the group consisting of foam capacity, foam stability, hardness, chewiness, guminess, springineness, less oily and retains more moisture. In some embodiments, the food item is a burger patty.

### 4. BRIEF DESCRIPTION OF THE SEVERAL VIEWS OF THE DRAWINGS

These and other features, aspects, and advantages of the present invention will become better understood with regard to the following description, and accompanying drawings, in which:

FIGS. 1A-1F are flow charts showing illustrative steps of methods of the present disclosure. FIG. 1A illustrates steps of an exemplary method of the present disclosure. FIG. 1B illustrates corresponding procedures of the illustrative steps in FIG. 1A. FIGS. 1C-1F illustrate steps of additional exemplary methods of the present disclosure. MF: microfiltration; UF-DF: ultrafiltration-diafiltration.

FIG. 2 is a graph showing the amount of protein (in grams/liter) recovered (in blue and bottom portion of each bar) and the amount of protein that was not recovered, i.e., proteins remaining in the supernatant (in orange and top portion of each bar), for proteins precipitated at various concentrations of ammonium sulfate.

FIG. 3 is a graph showing the percentage of protein recovered for proteins precipitated at various concentrations of ammonium sulfate, with pH at 4.5 throughout.

FIG. 4A is an SDS PAGE gel demonstrating the purification of the protein with certain process conditions; lanes 9 and 10 are duplicates of the supernatant and lanes 20 and 21 are the respective pellets resuspended in DI water to the initial volume. FIG. 4B includes chromatograms (top two images) for supernatant samples 9 and 10 of FIG. 4A and chromatogram (bottom two images) for precipitant samples 20 and 21.

FIG. 5 shows photograph of baked compositions made from the recovered protein of interest of Example 8.

FIG. 6 shows photograph of baked compositions made from the recovered protein of interest of Example 8.

FIG. 7 shows photograph of baked compositions made from the recovered protein of interest of Example 8.

FIG. 8 shows photograph of baked compositions made from the recovered protein of interest of Example 8.

FIGS. 9A-9B show photograph of compositions made from the recovered protein of interest in raw (FIG. 9A) and cooked form (FIG. 9B) of Example 9.

### 5. DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure relates to methods for increasing recovery and purity of a secreted protein of interest, recovered proteins from the methods, and uses of the proteins.

### 5.1. Methods of Recovering and Purifying a Secreted Protein of Interest

An aspect of the present disclosure is a method for increasing recovery and purity of a secreted protein of interest.

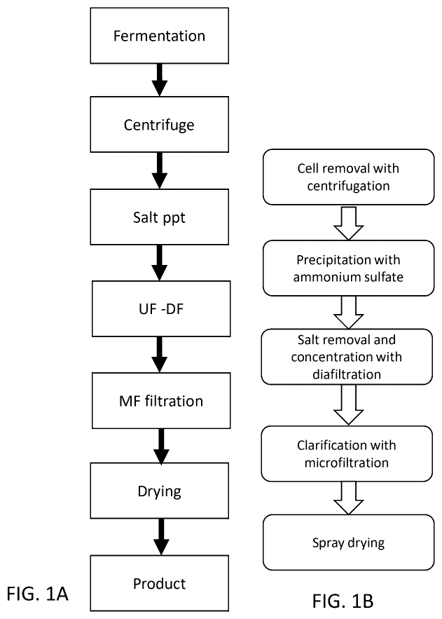

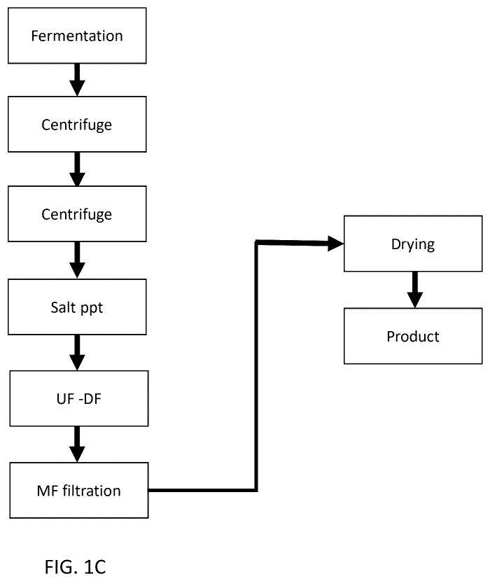

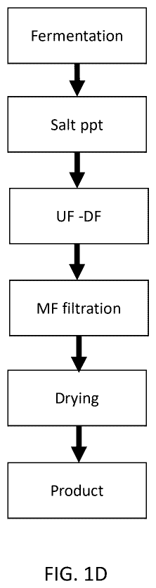

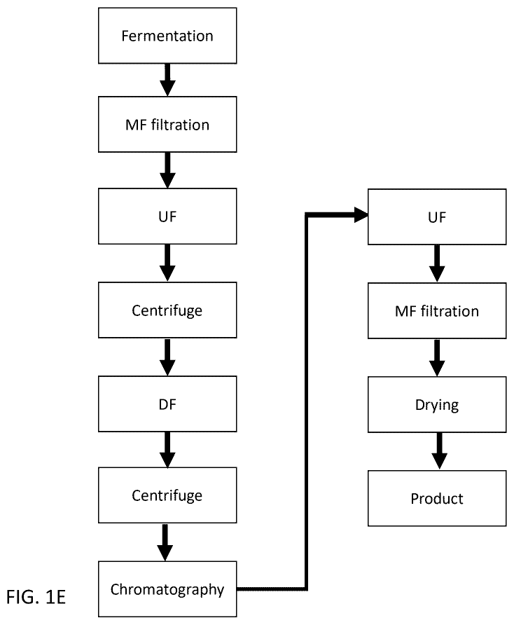

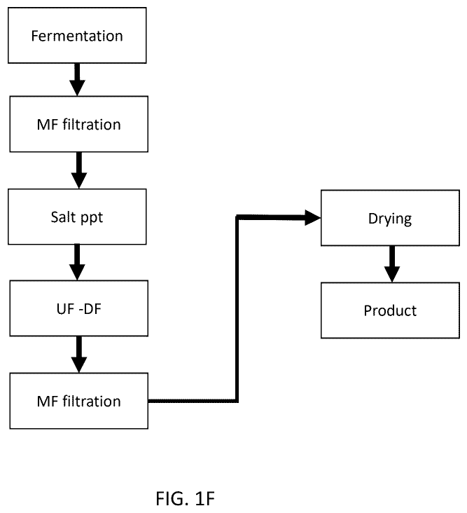

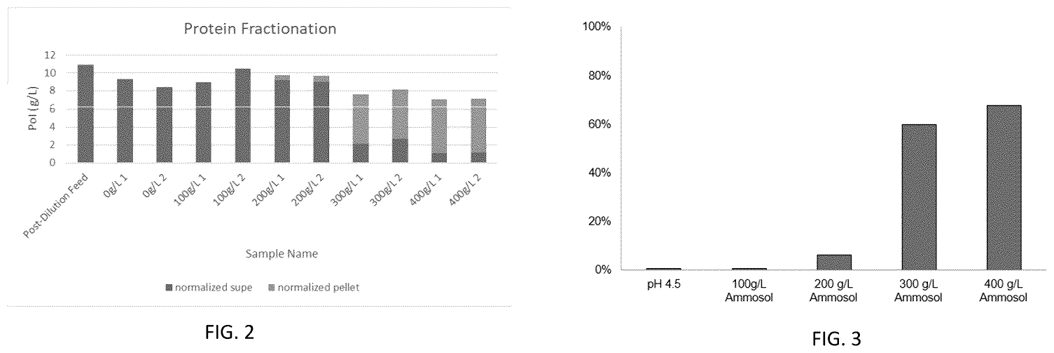

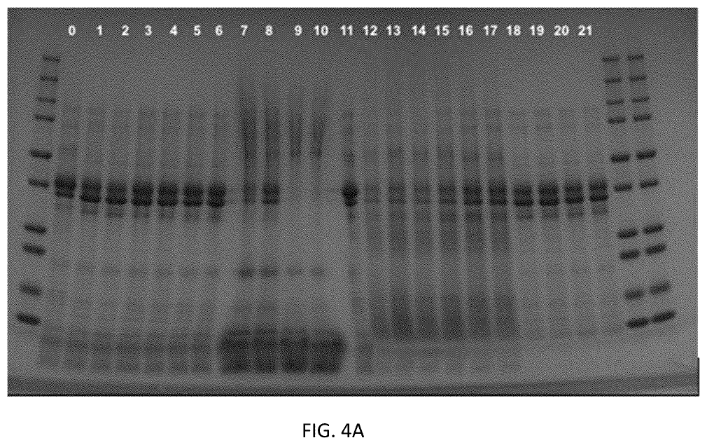

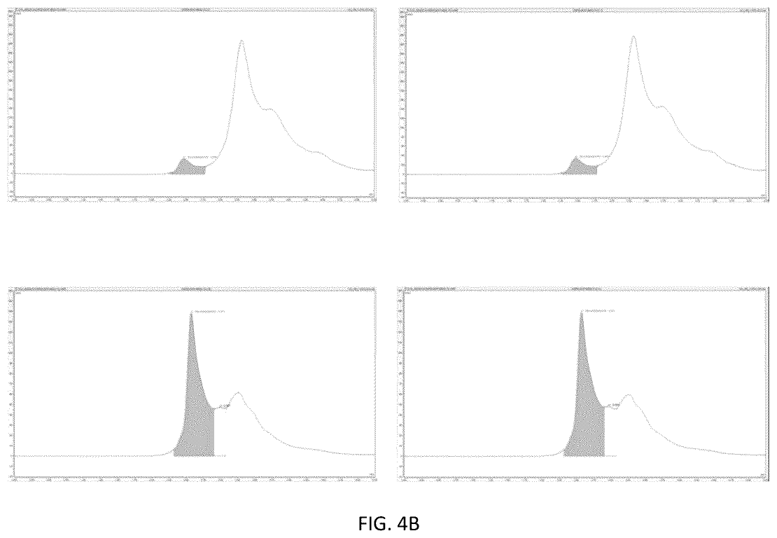

Exemplary methods are illustrated in FIGS. 1A-1F as described in Example 1.

In some embodiments, the method comprises obtaining recombinant fungal cells capable of expressing a secreted protein of interest and culturing the recombinant fungal cells under conditions that promote expression and secretion of the recombinant protein of interest into a culturing medium.

In some embodiments, the method comprises centrifuging the culturing medium and excluding or removing the recombinant fungal cells and other cellular components.

In some embodiments, the method comprises introducing ammonium sulfate to the culture medium to achieve an ammonium sulfate concentration of above 200 g/l.

In some embodiments, the method does not comprise microfiltering the centrifuged culturing medium to further remove any residual cell components prior to introducing ammonium sulfate to the culturing medium to achieve an ammonium sulfate concentration above 200 g/l, thereby precipitating the secreted protein of interest.

In some embodiments, the method comprises recovering the precipitated protein of interest. In some embodiments, the method comprises solubilizing the precipitated secreted protein of interest with water to obtain a solubilized protein of interest.

In some embodiments, the method comprises diafiltering and/or ultrafiltering the solubilized protein of interest.

In some embodiments, the method comprises microfiltering the diafiltered and/or ultrafiltered protein of interest.

In some embodiments, the method comprises drying the further microfiltered protein of interest, thereby obtaining a dried protein product.

### 5.1.1. Centrifugation to Remove the Recombinant Fungal Cells and Other Cell Components

An aspect of the present disclosure includes, after the protein of interest is secreted extracellularly by the recombinant fungal cells, the methods of the present disclosure include centrifuging the protein of interest within the culture medium in order to remove recombinant fungal cells and other cellular components and/or cell debris. Thus, the centrifuge can serve as a clarifier to clarify, dilute, and/or chill the protein of interest (e.g., centrate). For example, the method can include centrifuging the protein of interest within the culture medium at a particular speed and duration. Once the centrifugation is complete, the method includes removing the cells, cell particulates, cell debris and/or any solids and what remains is the protein of interest in culture medium.

In some embodiments, the centrifugation step occurs after the recombinant fungal cells express the protein of interest (e.g., after fermentation is complete). In some embodiments, the centrifugation step occurs after expression of the protein of interest but before the “salt precipitation” step of adding ammonium sulfate. In some embodiments, the centrifugation step occurs after the “salt precipitation” step of adding ammonium sulfate. In some embodiments, the method comprises one centrifugation step. In certain embodiments, the method comprises one or more centrifugation steps. In certain embodiments, the method comprises two or more centrifugation steps. In certain embodiments, the one or more centrifugation steps occur sequentially. In certain embodiments, the one or more centrifugation steps do not occur sequentially.

In certain embodiments, the method does not comprise a centrifugation step.

In some embodiments, the method comprises a step of centrifuging the culturing medium and excluding or removing the recombinant fungal cells and other cellular components prior to introducing ammonium sulfate.

### 5.1.2. Ammonium Sulfate

In some embodiments, the method comprises introducing ammonium sulfate to the culture medium. Ammonium sulfate is added to the retentate to precipitate the protein of interest.

In some embodiments, adding the ammonium sulfate to the culture medium comprises adding an amount of ammonium sulfate to achieve an ammonium sulfate concentration of above 200 g/l.

In some embodiments, the ammonium sulfate concentration is above or about 200 g/l, the ammonium sulfate concentration is above 300 g/l, or the ammonium sulfate concentration is above 400 g/l. The amount of recovered protein of interest is greater than the recovery that is obtained from a method that does not comprise adding ammonium sulfate to the culturing medium to achieve an ammonium sulfate concentration above 200 g/l.

In some embodiments, the ammonium sulfate concentration is above or about 100 g/l, the ammonium sulfate concentration is above 150 g/l, the ammonium sulfate concentration is above or about 200 g/l, the ammonium sulfate concentration is above or about 250 g/l, the ammonium sulfate concentration is above or about 300 g/l, the ammonium sulfate concentration is above or about 350 g/l, or the ammonium sulfate concentration is above 400 g/l. In some embodiments, the amount of recovered protein of interest is greater than the recovery that is obtained from a method that does not comprise adding ammonium sulfate to the culturing medium to achieve an ammonium sulfate concentration above 200 g/l.

In various embodiments, the ammonium sulfate is provided as a concentrated solution. In some case, the concentrated solution comprises about 30% w/v ammonium sulfate, about 35% w/v ammonium sulfate, about 40% w/v ammonium sulfate, about 45% w/v ammonium sulfate, about 50% w/v ammonium sulfate, about 55% w/v ammonium sulfate, about 60% w/v ammonium sulfate, about 65% w/v ammonium sulfate, about 70% w/v ammonium sulfate, or about 75% w/v ammonium sulfate. In some cases the concentrated solution comprises about 65% w/v ammonium sulfate. In some embodiments, the pH is about or below 4.75, is about or below 4.5, is about or below 4.25, is about or below 4.0, is about or below 3.75, is about or below 3.5, or is about or below 3.25 and wherein the ammonium sulfate concentration is about or above 200 g/l, the ammonium sulfate concentration is about or above 300 g/l, or the ammonium sulfate concentration about is or above 400 g/l. In various embodiments, the pH is about or below 4.75 and the ammonium sulfate concentration is about or above 300 g/l.

In embodiments, the pH is about or below 4.75 and the ammonium sulfate concentration is about or above 400 g/l.

In some embodiments, the pH is about or below 4.5 and the ammonium sulfate concentration is about or above 300 g/l. In various embodiments, the pH is about or below 4.5 and the ammonium sulfate concentration is about or above 400 g/l. In embodiments, the pH is about or below 4.25 and the ammonium sulfate concentration is about or above 300 g/l. In some embodiments, the pH is about or below 4.25 and the ammonium sulfate concentration is about or above 400 g/l. In various embodiments, the pH is about or below 4.0 and the ammonium sulfate concentration is about or above 300 g/l. In embodiments, the pH is about or below 4.0 and the ammonium sulfate concentration is about or above 400 g/l. In some embodiments, the pH is about or below 3.75 and the ammonium sulfate concentration is about or above 300 g/l. In various embodiments, the pH is about or below 3.75 and the ammonium sulfate concentration is about or above 400 g/l. In embodiments, the pH is about or below 3.5 and the ammonium sulfate concentration is about or above 300 g/l. In some embodiments, the pH is about or below 3.5 and the ammonium sulfate concentration is about or above 400 g/l. In various embodiments, the pH is about or below 3.25 and the ammonium sulfate concentration is about or above 300 g/l. In embodiments, the pH is about or below 3.25 and the ammonium sulfate concentration is about or above 400 g/l.

In various embodiments, the ammonium sulfate is added with moderate mixing to allow the salt to fully dissolve avoiding clumps and poor precipitation. A milky white precipitate will form, and hard agitation will cause foaming. Thus, moderate agitation and tank chilling (as compared to using an external heat exchanger and pump) is preferred. Precipitation of the protein of interest May take a few hours to almost a day, e.g., 2 hours, 3 hours, 4 hours, 5 hours, 6 hours, 7 hours, 8 hours, 9 hours, 10 hours, 11 hours, 12 hours, 13 hours, 14 hours, 16 hours, 17 hours, 18 hours, 19 hours, or 20 hours.

In some cases, after the protein is recovered by decanting or aspirating the ammonium sulfate-protein solution (slurry) from the container in which precipitation occurred. Any residual precipitate that is adhered to the container may be removed from the container by washing, e.g., with water, or by scraping the container, e.g., with a sterile implement. In some embodiments, the amount of recovered protein of interest is greater than the recovery that is obtained from a method that does not comprise reducing the pH of the culturing medium to about or below the pI of the protein of interest and does not comprise adding ammonium sulfate to the culturing medium to achieve an ammonium sulfate concentration above 200 g/l.

In some embodiments, the ammonium sulfate is added to the culturing medium after centrifugation. In some embodiments, the ammonium sulfate is added to the culturing medium after centrifugation but before ultrafiltering, diafiltering, and/or microfiltering.

Optionally, in some embodiments, the method comprises adding an agent to maintain the pH at about 4.5 to about 6.5 after centrifuging the culturing medium. Optionally, the method comprises adding an agent to maintain the pH at above the pI of the protein of interest after centrifuging the culturing medium. Optionally, wherein the method comprises recovering the precipitated protein of interest in a medium having a pH of about 4.5-6.5.

In some embodiments, the method comprises adding an agent to maintain the pH at about 4.5 to about 6.5 after centrifuging the culturing medium. The centrifuged culturing medium is maintained at about pH 6.0. The agent can be an acid such as phosphoric acid, e.g., 85% v/v phosphoric acid. The agent can be a base sodium hydroxide. The agent can be added before, after, or contemporaneously with introducing the ammonium sulfate.

### 5.1.3. Acid

In some embodiments, the method does not require adding an acid to the microfiltered culturing medium to reduce the pH to about or below pH 4.5 or the isoelectric point (pI) of the protein of interest and introducing ammonium sulfate. In some embodiments, the method does not consist of adding an acid to the microfiltered culturing medium to reduce the pH to about or below pH 4.5 or the isoelectric point (pI) of the protein of interest and introducing ammonium sulfate.

In some embodiments, the method comprises adding an acid to the microfiltered culturing medium to reduce the pH to about or below pH 4.5 or the isoelectric point (pI) of the protein of interest and introducing ammonium sulfate.

In some embodiments, the acid is phosphoric acid, e.g., 85% v/v phosphoric acid. In various embodiments, adding the acid occurs before introducing the ammonium sulfate. In embodiments, adding the acid occurs after introducing the ammonium sulfate. In some embodiments, adding the acid is contemporaneous with introducing the ammonium sulfate.

In some embodiments, the pH is reduced to a pH of 4.5 or less, 4 or less, 3.5 or less, 3 or less, or 2.5 or less.

### 5.1.4. Solubilizing the Precipitated Secreted Protein of Interest

In some embodiments, the method further comprises a step of solubilizing the precipitated secreted protein of interest, e.g., with water, e.g., DI water, to obtain a solubilized protein of interest. In some embodiments, the steps of solubilizing the protein of interest occurs prior to ultrafiltering the precipitated secreted protein of interest.

5.1.5. Diafiltering and/or Ultrafiltering the Precipitated Secreted Protein of Interest

In some embodiments, the method comprises diafiltering and/or ultrafiltering the precipitated secreted protein of interest. In some embodiments, diafiltering and/or ultrafiltering the precipitated secreted protein of interest occurs after recovery.

In some embodiments, diafiltering and/or ultrafiltering the precipitated secreted protein of interest occurs after microfiltering. In some embodiments, diafiltering and/or ultrafiltering the precipitated secreted protein of interest occurs after introducing ammonium sulfate to the culture medium. In some embodiments, diafiltering and/or ultrafiltering the precipitated secreted protein of interest occurs after microfiltering and introducing ammonium sulfate to the culture medium. In some embodiments, diafiltering and/or ultrafiltering the precipitated secreted protein of interest occurs after introducing ammonium sulfate but before microfiltration. In some embodiments, diafiltering and/or ultrafiltering the precipitated secreted protein of interest occurs before microfiltration. In some embodiments, diafiltering and/or ultrafiltering the precipitated secreted protein of interest occurs after microfiltration but before centrifugation of the protein of interest in the culture medium. In some embodiments, diafiltering and/or ultrafiltering the precipitated secreted protein of interest occurs after a first microfiltration step and introducing ammonium salt to the culture medium, but before a second microfiltration step.

Ultrafiltering and/or diafiltering steps can be used in the methods of the present disclosure based on existing ultrafiltration and diafiltering methods. In some embodiments, the method comprises ultrafiltering the protein of interest in the culture medium. In some embodiments, the method comprises diafiltering the protein of interest in the culture medium. In some embodiments, the method comprises ultrafiltering and diafiltering the protein of interest in the culture medium. In some embodiments, the method comprises ultrafiltering and diafiltering the protein of interest in the culture medium in sequential order. In some embodiments, the sequential order includes first ultrafiltering, followed by a diafiltering step. In some embodiments, the sequential order includes first diafiltering, followed by a ultrafiltering step.

In some embodiments, the ultrafiltration and/or diafiltration step is carried out using a 1 kDa membrane, a 2 kDa membrane, a 3 kDa membrane, a 4 kDa membrane, a 5 kdA membrane, a 6 kDa membrane, a 7 kDa membrane, a 8 kDa membrane, a 9 kDa membrane, or a 10 kDa membrane. In some embodiments, diafiltering comprises diafiltering the precipitated protein of interest using a membrane and/or a sodium chloride solution.

In some embodiments, the diafiltration step is carried out according to the parameters provided in Table 8. In some embodiments, the diafiltration step is configured to remove salts from the solution.

### 5.1.6. Microfiltering

In some embodiments, the method comprises microfiltering the protein of interest. In some embodiments, the method does not consist of microfiltering the protein of interest in the culture medium. In some embodiments, the method comprises microfiltering the protein of interest after introducing ammonium sulfate. In some embodiments, the method comprises microfiltering the protein of interest before introducing ammonium sulfate.

In some embodiments, the method comprises microfiltering the protein of interest after the ultrafiltering and/or diafiltering step. In some embodiments, the method comprises microfiltering the protein of interest before the ultrafiltering and/or diafiltering step. In some embodiments, the method comprises microfiltering the protein of interest after introducing ammonium sulfate and after the ultrafiltering and/or diafiltering step.

In some embodiments, the method comprises at one or more microfiltering steps. In some embodiments, the method comprises at two or more microfiltering steps. In some embodiments, the method comprises at three or more microfiltering steps. In some embodiments, the method comprises at four or more microfiltering steps.

In some embodiments, the method comprises at two or more microfiltering steps. In some embodiments, the method comprises microfiltering the protein of interest in the culture medium after the protein of interest is expressed by the recombinant fungal cell. In some embodiments, the method comprises microfiltering the protein of interest in the culture medium after the protein of interest is expressed by the recombinant fungal cell but before introducing ammonium sulfate. In some embodiments, the method comprises microfiltering the protein of interest in the culture medium after the centrifugation step. In some embodiments, the method comprises a first microfiltering step to microfilter the protein of interest in the culture medium, and a second microfiltering step after a ultrafiltering and/or diafiltering step. In some embodiments, the method comprises a first microfiltering step to microfilter the protein of interest in the culture medium, followed by introducing ammonium sulfate in the culture medium and ultrafiltering and/or diafiltering, and a second microfiltering step after the ultrafiltering and/or diafiltering step.

In some embodiments, the method comprises, after introducing ammonium salt to the culture medium, an ultrafiltering and/or diafiltering step, followed by one or more microfiltering steps. In certain embodiments, the method comprises two microfiltering steps following ultrafiltering and/or diafiltering. In certain embodiments, the method comprises three microfiltering steps following ultrafiltering and/or diafiltering.

In some embodiments, the microfiltering step comprises a filter capable of capturing fungal cells and other cellular components. In some embodiments, the size of the filter is at least 0.1 μm, at least 0.15 μm, at least 0.2 μm, at least 0.25 μm, at least 0.3 μm, at least 0.4 μm, or at least 0.5 μm. In some embodiments, the size of the filter is 0.1 μm or less, 0.15 μm or less, 0.2 μm, 0.25 μm or less, 0.3 μm or less, at least 0.4 μm or less, or 0.5 μm or less.

In some embodiments, one or more microfiltering steps is configured to remove any remaining cell debris. In some embodiments, one or more microfiltration steps are carried out using the parameters provided in Table 9.

In some embodiments, the centrifuged culturing medium is not microfiltered prior to introducing ammonium sulfate. In some embodiments, the centrifuged culturing medium is microfiltered prior to introducing ammonium sulfate.

### 5.1.7. Drying

In some embodiments, the method comprises a step of drying the further microfiltered protein of interest, thereby obtaining a dried protein product. In some embodiments, the method comprises spray drying. In some embodiments, the recovered protein of interest is spray dried at the following conditions: Inlet temperature: 165° C., Outlet temperature: 65-67° C., and Air inlet: 3 bar.

5.1.8. Purification Resin and/or Purification Column

In some embodiments, the method does not comprise or consist of use of a purification resin and/or a purification column. In some embodiments, the method does not comprise or consist of one or more chromatography steps.

In some embodiments, the method comprises use of a purification resin and/or a purification column.

In some embodiments, the method comprises one or more chromatography steps. In some embodiments, the one or more chromatography steps can include one or more of ion exchange chromatography, such as cation exchange chromatography and/or anion exchange chromatography.

In some embodiments, the one or more chromatography steps occurs after ultrafiltration and/or diafiltration steps. In some embodiments, the one or more chromatography steps occurs after a second centrifugation step as shown in FIG. 1E. In some embodiments, the one or more chromatography steps occurs in the order as shown in FIG. 1E.

### 5.1.9. Recovering Precipitated Protein of Interest

Aspects of the present disclosure include recovering the precipitated protein of interest.

In some embodiments, the method further comprises recovering the precipitated protein of interest in a medium having a pH of about 4.5-6.5. The precipitated protein of interest can be recovered at a pH of at least 4.5, at least 5.0, at least 5.5, or at least 6.0; or at a pH of about 4.5, about 5.5, or about 6.0.

In some embodiments, recovery of the protein of interest is at least about 40% w/w, is at least about 45% w/w, is at least about 50% w/w, is at least about 55% w/w, is at least about 60% w/w for the final dried product, is at least about 65% w/w for the final dried product, is at least about 70% w/w for the final dried product, is at least about 75% w/w for the final dried product, is at least about 80% w/w for the final dried product, is at least about 85% w/w for the final dried product, or is at least about 90% w/w for the final dried product, wherein the recovery is the weight of the protein of interest recovered in the final product relative to the weight of the protein of interest prior to or following introducing ammonium sulfate.

The recovered protein of interest is a food protein used as nutritional, dietary, digestive, supplements, such as in food products and feed products. The food protein is a plant protein, or an animal protein such as an egg white protein selected from ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

In some embodiments, the egg white protein is an ovalbumin (OVA) that comprises the amino acid sequence of a chicken OVA, a goose OVA, a quail OVA, an ostrich OVA, or a duck OVA. In some embodiments, the egg white protein has at least 70%, 75%, 80%, 85%, 90%, 95%, 96%, 97%, 98%, or 99% sequence identity to one of SEQ ID NO: 1 to SEQ ID NO: 74 as described in Table 1.

In some embodiments, the recovered recombinant OVA (rOVA) is is devoid of N-linked galactose units, lacks an N-terminal methionine, glycosylated, or non-glycosylated.

The recovered protein of interest is suitable for use in a food product such as baked food item or a non-meat food item. The food product has an additional characteristic equivalent to or better than a similar baked item made with a natural egg white or a natural whole egg, wherein the characteristic is selected from the group consisting of foam capacity, foam stability, hardness, chewiness, guminess, and springineness. The baked food product is selected from the group consisting of a cake, pound cake, cookie, bagel, biscuit, bread, muffin, cupcake, scone, pancake, macaroon, choux pastry, and soufflé. The non-meat food item is a burger patty. The burger patty is less oily and maintains more moisture than a similar burger made with natural egg white or natural whole egg.

In some embodiments, the baked food item or the non-meat food item made of the recovered rOVA is vegan or vegetarian.

### 5.1.10. Fungal Cells

In some embodiments, the fungal cells are of the species selected from Agaricus bisporus; Agaricus spp.; Aspergillus awamori; Aspergillus fumigatus; Aspergillus nidulans; Aspergillus niger; Aspergillus oryzae; Aspergillus oryzae; Aspergillus spp.; Colletotrichum gloeosporiodes; Colletotrichum spp.; Endothia parasitica; Endothia spp.; Fusarium graminearum; Fusarium solani; Fusarium spp.; Komagatella pastoris; Komagatella phaffi; Mucor miehei; Mucor pusillus; Mucor spp.; Myceliophthora spp.; Myceliophthora thermophila; Neurospora crassa; Neurospora spp.; Penicillium (Talaromyces) emersonii; Penicillium camemberti; Penicillium canescens; Penicillium chrysogenum; Penicillium funiculosum; Penicillium purpurogenum; Penicillium roqueforti; Penicillium spp.; Pichia angusta; Pichiapastoris; Pichia pastoris; Pichia Pastoris “MutS” strain (Graz University of Technology (CBS7435MutS) or Biogrammatics (BG11)); Pichia spp.; Pleurotus ostreatus; Pleurotus spp.; Rhizomucor miehei; Rhizomucor pusillus; Rhizomucor spp.; Rhizopus arrhizus; Rhizopus oligosporus; Rhizopus oryzae; Rhizopus spp.; Trichoderma altroviride; Trichoderma reesei; Trichoderma spp.; Trichoderma vireus; Yarrowia lipolytica; and Yarrowia spp.

In some embodiments, the fungal cells are Aspergillus cells of the species Aspergillus spp., Aspergillus awamori, Aspergillus fumigatus, Aspergillus nidulans, Aspergillus niger, or Aspergillus oryzae. In some embodiments, the Aspergillus cells are Aspergillus niger cells.

### 5.1.11. Exemplary Methods for Recovering Protein of Interest

FIGS. 1A-1F illustrate various methods for recovering protein of interest. FIG. 1A is a flow chart that illustrates illustrative steps of methods of the present disclosure. FIG. 1B illustrates procedures of corresponding steps in FIG. 1A.

The steps of the illustrated method include: obtaining recombinant fungal cells capable of expressing a secreted protein of interest; culturing the recombinant fungal cells under conditions that promote expression and secretion of the recombinant protein of interest into a culturing medium; centrifuging the culturing medium and excluding or removing the recombinant fungal cells and other cellular components; without microfiltering the centrifuged culturing medium to further remove any residual cell components prior to introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate. See e.g., FIGS. 1A and 1C.

Referring to FIG. 1B, the culture medium was centrifuged to remove recombinant fungal cells and other cellular components (e.g. “centrifuge” step of FIG. 1A). Ammonium sulfate (200 g/l; 65% w/v) was added to the supernatant containing the protein of interest released from the recombinant fungal cells (e.g., “salt ppt” step of FIG. 1A). The precipitated protein of interest was resuspended to remove excess salt via diafiltering (DF) and/or ultrafiltering (UF) (e.g., “UF-DF” step of FIG. 1A) the solubilized protein of interest, which was then diluted to a concentration of 50 g/L. The protein of interest was further microfiltered (0.2 μm) (“MF filtration” step of FIG. 1A) to improve clarification, followed by drying (e.g., spray drying) to produce a dried protein product.

In some cases, one or more of the above steps are performed one or more times. As shown in FIG. 1C, the method comprises obtaining recombinant fungal cells capable of expressing a secreted protein of interest; culturing the recombinant fungal cells under conditions that promote expression and secretion of the recombinant protein of interest into a culturing medium; centrifuging the culturing medium and excluding or removing the recombinant fungal cells and other cellular components for at least two times; introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below the isoelectric point (pI) of the protein of interest and introducing ammonium sulfate.

In some cases, one more of the above steps is omitted.

For example, in some embodiments, the method may conclude when the protein of interest is precipitated and recovered. In this case, the method comprises steps of: obtaining recombinant fungal cells capable of expressing a secreted protein of interest; culturing the recombinant fungal cells under conditions that promote expression and secretion of the recombinant protein of interest into a culturing medium; introducing ammonium sulfate to the culturing medium to achieve an ammonium sulfate concentration above 200 g/l, thereby precipitating the secreted protein of interest; and recovering the precipitated protein of interest.

FIG. 1D illustrates an alternative method. As in FIG. 1D, the method comprises steps of: obtaining recombinant fungal cells capable of expressing a secreted protein of interest; culturing the recombinant fungal cells under conditions that promote expression and secretion of the recombinant protein of interest into a culturing medium; introducing ammonium sulfate to the culturing medium to achieve an ammonium sulfate concentration above 200 g/l, thereby precipitating the secreted protein of interest; and recovering the precipitated protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

As in FIG. 1E, the method comprises steps of: obtaining recombinant fungal cells capable of expressing a secreted protein of interest; culturing the recombinant fungal cells under conditions that promote expression and secretion of the recombinant protein of interest into a culturing medium; microfiltering the centrifuged culturing medium to further remove any residual cell components; ultrafiltering the solubilized protein of interest; centrifuging the culturing medium to remove the recombinant fungal cells and other cellular components; diafiltered the protein of interested; centrifuging the culturing medium to remove the recombinant fungal cells and other cellular components; testing purity of the protein of interest with chromatography; ultrafiltering the solubilized protein of interest; microfiltering the centrifuged culturing medium to further remove any residual cell components; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

As shown in FIG. 1F, the method comprises steps of: obtaining recombinant fungal cells capable of expressing a secreted protein of interest; culturing the recombinant fungal cells under conditions that promote expression and secretion of the recombinant protein of interest into a culturing medium; microfiltering the centrifuged culturing medium to further remove any residual cell components; introducing ammonium sulfate to the culturing medium to achieve an ammonium sulfate concentration above 200 g/l, thereby precipitating the secreted protein of interest; and recovering the precipitated protein of interest; diafiltering and/or ultrafiltering the solubilized protein of interest; microfiltering the diafiltered and/or ultrafiltered protein of interested; microfiltering the centrifuged culturing medium to further remove any residual cell components; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

An illustrative process scale up of the method is further described in Example 6.

### 5.2. Protein of Interest

Any protein of interest that may be recombinantly expressed and secreted by a fungal cell may be used in methods of the present disclosure. Proteins that can be recombinantly expressed by a fungal cell but cannot normally be secreted by the fungal cell may still be recovered by methods of the present disclosure; in these cases, the protein of interest is modified (e.g., by genetic manipulation of its DNA code) to express a signal that permits its secretion from the fungal cells. Such secretion signals are well-known in the art and choice of signal (or DNA encoding the signal) can be selected based on the fungal cell used and/or the specific protein of interest.

Another aspect of the present disclosure is a powdered composition comprising any herein-disclosed recovered protein of interest.

Yet another aspect of the present disclosure is a liquid composition comprising a solvent suitable for animal or human consumption and a powdered composition comprising any herein-disclosed recovered protein of interest.

In some cases, the protein of interest is a food protein, e.g., which is used as nutritional, dietary, digestive, supplements, such as in food products and feed products. The food protein may be a plant protein or may be an animal protein.

The animal protein may be an egg white protein, e.g., selected from ovalbumin, ovomucoid, ovotransferrin, lysozyme, ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

In some cases, the egg white protein is an ovalbumin (OVA) that comprises the amino acid sequence of a chicken OVA, a goose OVA, a quail OVA, an ostrich OVA, or a duck OVA. The egg white protein may have at least 70%, 75%, 80%, 85%, 90%, 95%, 96%, 97%, 98%, or 99% sequence identity to any one of SEQ ID NOs: 1-74. An rOVA can be a non-naturally occurring variant of an OVA. Such variant can comprise one or more amino acid insertions, deletions, or substitutions relative to a native OVA sequence. Such a variant can have at least 70%, 75%, 80%, 85%, 90%, 95%, 96%, 97%, 98%, or 99% sequence identity to any one of SEQ ID NOs: 1-74. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software, with BLAST being the preferable alignment algorithm. Those skilled in the art can determine appropriate parameters for measuring alignment, including any algorithms needed to achieve maximal alignment over the full-length of the sequences being compared.

Depending on the host organism used to express the rOVA, the rOVA can have a glycosylation, acetylation, or phosphorylation pattern different from wildtype OVA. For example, the rOVA herein may or may not be glycosylated, acetylated, or phosphorylated. An rOVA may have an avian, non-avian, microbial, non-microbial, mammalian, or non-mammalian glycosylation, acetylation, or phosphorylation pattern.

In some cases, rOVA may be deglycosylated (e.g., chemically, enzymatically, Endo-H, PNGase F, O-Glycosidase, Neuraminidase, β1-4 Galactosidase, β-N-acetylglucosaminidase), deacetylated (e.g., protein deacetylase, histone deacetylase, sirtuin), or dephosphorylated (e.g., acid phosphatase, lambda protein phosphatase, calf intestinal phosphatase, alkaline phosphatase). Deglycosylation, deacetylation or dephosphorylation may produce a protein that is more uniform or is capable of producing a composition with less variation.

The present disclosure contemplates modifying glycosylation of the recombinant OVA to alter or enhance one or more functional characteristics of the protein and/or its production. In some embodiments, the change in rOVA glycosylation can be due to the host cell glycosylating the rOVA. In some embodiments, rOVA has a glycosylation pattern that is not identical to a native ovalbumin (nOVA), such as a nOVA from chicken egg. In some embodiments, rOVA is treated with a deglycosylating enzyme before it is used as an ingredient in an rOVA composition, or when rOVA is present in a composition. In some embodiments, the glycosylation of rOVA is modified or removed by expressing one or more enzymes in a host cell and exposing rOVA to the one or more enzymes. In some embodiments, rOVA and the one or more enzymes for modification or removal of glycosylation are co-expressed in the same host cell.

Native ovalbumin (nOVA), such as isolated from a chicken or another avian egg, has a highly complex branched form of glycosylation. The glycosylation pattern comprises N-linked glycan structures such as N-acetylglucosamine units, galactose and N-linked mannose units. In some cases, the rOVA for use in a herein disclosed consumable composition and produced using the methods described herein has a glycosylation pattern which is different from the glycosylation pattern of nOVA. For example, when rOVA is produced in a Pichia sp., the protein may be glycosylated differently from the nOVA and lack galactose units in the N-linked glycosylation. The glycosylation patterns of rOVA produced by P. pastoris have a complex branched glycosylation pattern. In some embodiments of the compositions and methods disclosed herein, rOVA is treated such that the glycosylation pattern is modified from that of nOVA and also modified as compared to rOVA produced by a Pichia sp. without such treatment. In some cases, the rOVA lacks glycosylation.

The molecular weight or rOVA may be different as compared to nOVA. The molecular weight of the protein may be less than the molecular weight of nOVA or less than rOVA produced by the host cell where the glycosylation of rOVA is not modified. In embodiments, the molecular weight of an rOVA may be between 40 kDa and 55 kDa. In some cases, an rOVA with modified glycosylation has a different molecular weight, such as compared to a native OVA (as produced by an avian host species) or as compared to a host cell that glycosylates the rOVA, such as where the rOVA includes N-linked mannosylation. In some cases, the molecular weight of rOVA is greater than the molecular weight of the rOVA that is completely devoid of post-translational modifications. or an rOVA that lacks all forms of N-linked glycosylation. In some embodiments, the molecular weight of an rOVA is 30 kDa or more, 35 kDa or more, 40 kDa or more, 45 kDa or more, 50 kDa or more, 55 kDa or more, 60 kDa or more, 65 kDa or more, or 70 kDa or more.

The compositions and methods provided herein contain fermentation-derived ovalbumin, produced through recombinant technology, i.e., a recombinant ovalbumin (rOVA). The compositions and methods for making compositions comprising rOVA can increase the protein content of a consumable or food ingredient, and also provide functional features for use in the preparation of food ingredients and consumable food products for animal and human ingestion.

In some embodiments, the rOVA provides one or more functional characteristics such as of gelling, foaming, whipping, fluffing, binding, springiness, aeration, coating, film forming, emulsification, browning, thickening, texturizing, humectant, clarification, and cohesiveness. The rOVA with such feature(s) can be a food ingredient that provides for production of an egg-less or animal-free food ingredient or food product.

As used herein “native” in the context of native egg white, native egg protein, native ovalbumin and native egg, refers to the egg white, egg protein, ovalbumin or whole egg, respectively, produced by an animal or collected from an animal, in particular an egg-laying animal such as a bird. The rOVA and compositions containing rOVA can be used in food ingredients and food products, such that the ingredient or product does not contain any native egg white, native egg protein, native ovalbumin or native egg. In some cases, the ingredients or food products made using rOVA do not include any egg-white proteins other than rOVA. The rOVA and compositions containing rOVA can be used in food ingredients and food products, such that the ingredient or product does not contain any animal products.

In some embodiments, the rOVA can (alone or with other ingredients) substitute for the use of whole egg or egg white in the production of a food product. In some embodiments, the feature(s) provided by the rOVA is substantially the same or better than the same characteristic provided by a native egg white or native egg. For example, the rOVA and compositions containing rOVA can have gelling, foaming, whipping, fluffing, binding, springiness, aeration, coating, film forming, emulsification, browning, thickening, texturizing, preserving moisture (humectant), clarification, and cohesiveness, improved color, such as a whiter color, as compared to native egg white or native whole egg and compositions made with native egg white.

5.3. Food Ingredients and Food Products with rOVA

Food ingredients and food products disclosed herein include compositions that comprise, consists essentially of, or consist of rOVA, where rOVA provides at least one functional feature to the composition, food ingredient, or food product. In some cases, at least one functional feature provided by the rOVA is comparable or substantially similar to a native egg or egg white or native OVA (nOVA). For instance, it may provide any one of gelling, foaming, whipping, fluffing, binding, springiness, aeration, coating, film forming, emulsification, browning, thickening, texturizing, preserving moisture (humectant), clarification, and cohesiveness comparable to a whole egg, egg-white or nOVA composition. In some embodiments, the at least one functional feature is provided by or provided substantially by the inclusion of rOVA in the food ingredient or food product, for example, in the absence of any other whole egg proteins or egg white proteins.

Such compositions can include rOVA in an amount between 0.1% and 25% on a weight/weight (w/w) or weight/volume (w/v) basis. rOVA may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% on a weight/weight (w/w) or weight/volume (w/v) basis. These concentrations can be based on the dry weight of the composition. Additionally, or alternatively, the concentration of rOVA in such compositions is at most 30%, 20%, 15%, 10%, 5%, 4%, 3%, 2% or 1% on a w/w or w/v basis. In some embodiments, the rOVA in the food ingredient or food product can be at a concentration range of 0.1%-20%, 1%-20%, 0.1%-10%, 1%-10%, 0.1%-5%, 1%-5%, 2-10%, 4-8%, 4-10%, 4-12%, 0.1%-2%, 1%-2% or 0.1-1%.

Provided herein are consumable food compositions and methods of making such compositions where rOVA provides at least one feature of whole egg or egg-whites to a consumable food composition. In some embodiments, rOVA is added to a consumable food composition to increase the protein content, such as for added nutrition. In some embodiments, rOVA is present in the consumable food composition between about 1% and about 40% on a weight per total weight (w/w) and/or weight per total volume (w/v) of composition basis. For example, in a composition of 100 ml, rOVA is present at 30 g and the rOVA is thus at a 30% concentration (w/v) or for example, in a composition of 100 g, rOVA is present at 30 g and the rOVA is thus at a 30% concentration (w/w). In some embodiments, the concentration of rOVA is or is about 0.5%, 1%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% on a w/w and/or w/v of composition basis. In some embodiments, the rOVA is present at a concentration of or of about 0.5-1%, 1-5%, 2-8%, 4-8%, 2-12%, 4-12%, 5-10%, 10-15%, 15-20%, 20-25%, 25-30% or rOVA is present concentration greater than 1%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% w/w and/or w/v.

A consumable product can include one or more other proteins, such as a non-OVA protein or a non-recombinant protein. The rOVA can increase amount of protein content in a consumable product, and/or provide one or more egg-white like features. For example, the consumable composition can include a whey protein, a pea protein, a soy protein, an almond protein, an oat protein, a flax seed protein, a vegetable protein, or an egg-white protein. The consumable protein may include an extruded plant protein or a non-extruded plant protein. In some cases, the one or more other proteins can comprise OVA having an amino acid sequence naturally found in a bird or a reptile.

In some embodiments, the compositions and methods for making compositions have an egg-white like property and increase the protein content in the composition. In some embodiments, the compositions and methods for making compositions with an egg-white like property increase the protein content, while not adversely affecting the stability, or one or more sensory qualities of the composition.

In some embodiments, the consumable food compositions and methods for making consumable food compositions comprise rOVA and the addition of rOVA generates an egg-white like composition. The consumable food composition may be a finished product or an ingredient for making a finished product, e.g., a liquid or a powdered rOVA composition.

rOVA protein may be used on its own or in combination with other components to form a composition. In some embodiments, rOVA is used as an ingredient to form a composition and the rOVA ingredient (or rOVA starting composition to be added) may contain about or at least about 10%, 20%, 30%, 40%, 50%, 55%, 60%, 65%, 70%, 75%, 80%, 85%, 90%, 95%, 96%, 97%, 98%, or 99% rOVA by weight per total weight (w/w) and/or weight per total volume (w/v). In some cases, a composition described herein may contain up to about 10%, 20%, 30%, 40%, 50%, 55%, 60%, 65%, 70%, 75%, 80%, 85%, 90%, 95%, 96%, 97%, 98%, or 99% rOVA by w/w or w/v. In some embodiments, about or at least about 10%, 20%, 30%, 40%, 50%, 55%, 60%, 65%, 70%, 75%, 80%, 85%, 90%, 95%, 96%, 97%, 98%, or 99% of the protein in a composition is rOVA by weight per total weight (w/w) and/or weight per total volume (w/v). In some cases, up to or about 10%, 20%, 30%, 40%, 50%, 55%, 60%, 65%, 70%, 75%, 80%, 85%, 90%, 95%, 96%, 97%, 98%, or 99% of the protein in a composition is rOVA by w/w or w/v.

In some embodiments, a composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g total protein per 100 mL liquid (e.g., water). In some cases, a composition described herein contains total protein at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder).

In some embodiments, a composition described herein contains rOVA at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, or 75 g per 100 mL liquid (e.g., water). In some cases, a composition described herein contains rOVA at a concentration of about or at least 5, 6, 7, 8, 9, 10, 11, 12, 13, 13.2, 14, 15, 16, 17, 18, 19, 20, 21, 22, 23, 24, 25, 30, 35, 40, 45, 50, 55, 60, 65, 70, 75, 80, 85, 90, 95, or 100 g total protein per 100 g composition (e.g., powder).

In some embodiments, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g total protein per 100 mL liquid (e.g., water). In some cases, a composition described herein contains total protein at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g total protein per 100 g composition (e.g., powder).

In some embodiments, a composition described herein contains rOVA at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g per 100 mL liquid (e.g., water). In some cases, a composition described herein contains rOVA at a concentration of about or at least 0.1, 0.2, 0.3, 0.5, 0.7, 1.0, 1.2, 1.5, 1.7, 2.0, 2.2, 2.5, 2.7, 3.0, 3.2, 3.5, 3.7, 4.0, 4.2, 4.5, 4.7 or 5 g per 100 g composition (e.g., powder).

In some embodiments, the rOVA consumable composition is a liquid composition. In such cases, the concentration of rOVA in the liquid composition may be between 0.1% to 90%. The concentration of rOVA in the liquid composition may be at least 0.1%. The concentration of rOVA in the liquid composition may be at most 90%. The concentration of rOVA in the liquid composition may be from 0.1% to 1%, 0.1% to 5%, 0.1% to 10%, 0.1% to 15%, 0.1% to 20%, 0.1% to 25%, 0.1% to 30%, 0.1% to 35%, 0.1% to 40%, 1% to 5%, 1% to 10%, 1% to 15%, 1% to 20%, 1% to 25%, 1% to 30%, 1% to 35%, 1% to 40%, 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, or 90% to 95% in weight per total volume (w/v). The concentration of rOVA in the liquid composition may be about 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of rOVA in the liquid composition may be at least 0.1%, 1%, 5%, 10%, 15%, 20%, 25%, 30%, 35%, 40%, 45%, 50%, 55%, 60%, 65%, 70%, 75%, 80%, 85%, 90%, or 95% w/v. The concentration of rOVA in the liquid composition may be at most 1%, 5%, 10%, 15%, 20%, 25%, 30%, 35% 40%, 45%, 50%, 55%, 60%, 65%, 70%, 75%, 80%, 85%, 90%, or 95% w/v. In some embodiments, rOVA is the sole protein in the liquid composition. In other embodiments, a liquid composition comprises proteins other than rOVA.

In some embodiments, the rOVA consumable composition is a solid composition. In such cases, the concentration of rOVA in the solid composition may be between 0.1% to 70%. The concentration of rOVA in the solid composition may be at least 0.1%. The concentration of rOVA in the solid composition may be at most 70%. The concentration of rOVA in the solid composition may be 0.1% to 1%, 0.1% to 10%, 0.1% to 20%, 0.1% to 30%, 0.1% to 40%, 0.1% to 50%, 0.1% to 60%, 0.1% to 70%, 1% to 10%, 1% to 20%, 1% to 30%, 1% to 40%, 1% to 50%, 1% to 60%, 1% to 70%, 10% to 20%, 10% to 30%, 10% to 40%, 10% to 50%, 10% to 60%, 10% to 70%, 20% to 30%, 20% to 40%, 20% to 50%, 20% to 60%, 20% to 70%, 30% to 40%, 30% to 50%, 30% to 60%, 30% to 70%, 40% to 50%, 40% to 60%, 40% to 70%, 50% to 60%, 50% to 70%, or 60% to 70% weight per total weight (w/w) and/or weight per total volume (w/v). The concentration of rOVA in the solid composition may be 0.1%, 1%, 10%, 20%, 30%, 40%, 50%, 60%, or 70% w/w or w/v. The concentration of rOVA in the solid composition may be at least 0.1%, 1%, 10%, 20%, 30%, 40%, 50% or 60% w/w or w/v. The concentration of rOVA in the solid composition may be at most 1%, 10%, 20%, 30%, 40%, 50%, 60%, or 70% w/w or w/v.

In some embodiments, the rOVA consumable composition is a powdered composition. In such cases, the concentration of rOVA in the powder composition may be between 15% to 99% weight per total weight (w/w) and/or weight per total volume (w/v). The concentration of rOVA in the powder composition may be at least 15% w/w or w/v. In embodiments, the concentration of rOVA in the powder composition may be at most 99% w/w or w/v. The concentration of rOVA in the powder composition may be 15% to 30%, 15% to 45%, 15% to 60%, 15% to 75%, 15% to 80%, 15% to 85%, 15% to 90%, 15% to 95%, 15% to 99%, 30% to 45%, 30% to 60%, 30% to 75%, 30% to 80%, 30% to 85%, 30% to 90%, 30% to 95%, 30% to 99%, 45% to 60%, 45% to 75%, 45% to 80%, 45% to 85%, 45% to 90%, 45% to 95%, 45% to 99%, 60% to 75%, 60% to 80%, 60% to 85%, 60% to 90%, 60% to 95%, 60% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w or w/v. The concentration of rOVA in the powder composition may be about 15%, 30%, 45%, 60%, 75%, 80%, 85%, 90%, 95%, or 99% w/w or w/v. The concentration of rOVA in the powder composition may be at least 15%, 30%, 45%, 60%, 75%, 80%, 85%, 90% or 95% w/w or w/v. The concentration of rOVA in the powder composition may be at most 30%, 45%, 60%, 75%, 80%, 85%, 90%, 95%, or 99% w/w or w/v. In some embodiments, rOVA is the sole protein in the powder composition. In other embodiments, a powder composition comprises proteins other than rOVA.

In some cases, a powder composition may be a concentrate which comprises at least 70% rOVA w/w. In some cases, a powder composition may be a concentrate which comprises at least 80% rOVA w/w. In some cases, a powder composition may be an isolate which comprises at least 90% rOVA w/w. In some cases, a powder composition may be an isolate which comprises at least 95% rOVA w/w.

In some embodiments, the rOVA consumable composition is a concentrated liquid composition. In such cases, the concentration of rOVA in the concentrated liquid composition may be between 10% to 60% weight per total weight (w/w) and/or weight per total volume (w/v). The concentration of rOVA in the concentrated liquid may be at least 10% w/w or w/v. The concentration of rOVA in the concentrated liquid may be at most 60% w/w or w/v. The concentration of rOVA in the concentrated liquid may be 10% to 20%, 10% to 30%, 10% to 40%, 10% to 50%, 10% to 60%, 20% to 30%, 20% to 40%, 20% to 50%, 20% to 60%, 30% to 40%, 30% to 50%, 30% to 60%, 40% to 50%, 40% to 60%, or 50% to 60% w/w or w/v. The concentration of rOVA in the concentrated liquid may be about 10%, 20%, 30%, 40%, 50%, or 60% w/w or w/v. The concentration of rOVA in the concentrated liquid may be at least 10%, 20%, 30%, 40% or 50% w/w or w/v. The concentration of rOVA in the concentrated liquid may be at most 20%, 30%, 40%, 50%, or 60% w/w or w/v. The liquid may include any consumable solvent, e.g., water, dairy, oil, or other cooking base.

In some embodiments, the rOVA consumable composition is a prepared food for example, as a baked good, a salad dressing, an egg-like dish (such as an egg-patty or scramble), a dessert or dairy-like product or a meat-analog (such as a vegan meat patty, sausage or hot dog). Such compositions can include rOVA in an amount between 0.1% and 20% on a weight/weight (w/w) or weight/volume (w/v) basis. rOVA may be present at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% on a weight/weight (w/w) or weight/volume (w/v) basis. Additionally, or alternatively, the concentration of rOVA in such compositions is at most 30%, 20%, 15%, 10%, 5%, 4%, 3%, 2% or 1% on a w/w or w/v basis. In some embodiments, the rOVA in the food ingredient or food product can be at a concentration range of 0.1%-20%, 1%-20%, 0.1%-10%, 1%-10%, 0.1%-5%, 1%-5%, 0.1%-2%, 1%-2% or 0.1-1%.

5.4. Features and Characteristics of rOVA and Food Ingredients and Food Products Containing rOVA

The rOVA containing compositions herein can provide one or more functional features to food ingredients and food products. In some embodiments, the rOVA provides a nutritional feature such as protein content, protein fortification and amino acid content to a food ingredient or food product. The nutritional feature provided by rOVA in the composition may be comparable or substantially similar to an egg, egg white or native OVA (nOVA). The nutritional feature provided by rOVA in the composition may be better than that provided by a native whole egg or native egg white. In some cases, rOVA provides the one or more functional features of egg-white in absence of any other egg-white proteins.

rOVA compositions disclosed herein can provide foaming and foam capacity to a composition. For example, rOVA can be used for forming a foam to use in baked products, such as cakes, for meringues and other foods where rOVA can replace egg white to provide foam capacity. In some cases, rOVA provides foaming and foam capacity of egg-white in absence of any other egg-white proteins.

A composition comprising rOVA may have a foam height greater than a foam height of an egg white or a composition comprising nOVA. In some cases, a composition comprising rOVA may have a foam height of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. In some cases, a composition comprising rOVA may have a foam height of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Substitute egg whites may include products such as aquafaba, chia seeds, flax seeds, starches; apple sauce, banana puree; condensed milk, etc. which are commonly used as egg white substitutes.

A composition comprising rOVA may have a foam stability greater than a foam stability of an egg white, nOVA compositions or a substitute egg white. In some cases, a composition comprising rOVA may have a foam stability of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white or a substitute egg white. In some cases, a composition comprising rOVA may have a foam stability of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. Foam stability may be calculated by measuring drainage of a foamed solution. The drainage may be measured in 10-minute increments for 30 minutes to gather data for foam stability. The drained volume after 30 minutes may be compared to the initial liquid volume (5 mL) for instance, foam Stability (%): (Initial volume−drained volume)/initial volume*100.

A composition comprising rOVA may have a foam capacity greater than a foam capacity of an egg white, nOVA compositions or a substitute egg white. In some cases, a composition comprising rOVA may have a foam capacity of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA or a substitute egg white. In some cases, a composition comprising rOVA may have a foam capacity of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white, nOVA compositions or a substitute egg white. Foam capacity may be determined by measuring the initial volume of foam following the whipping and compare against the initial volume of 5 mL. Foam Capacity (%)=(volume of foam/initial volume)*100.

A liquid composition may foam faster than a composition comprising egg whites, nOVA or a substitute egg white. In some cases, an rOVA composition foams at least 1%, 5%, 10%, 15%, 20%, 25%, 30%, 35%, 40%, 45%, 50%, 55%, 60%, 65%, 70%, 75%, 80%, 85%, 90%, 95%, 100%, faster than an egg white, nOVA or substitute egg-white composition. In some cases, an rOVA composition foams up to 1%, 5%, 10%, 15%, 20%, 25%, 30%, 35%, 40%, 45%, 50%, 55%, 60%, 65%, 70%, 75%, 80%, 85%, 90%, 95%, 100% faster than an egg white, nOVA or substitute egg-white composition.

A composition comprising rOVA may have a gel strength greater than a gel strength of an egg white, nOVA composition or an egg white substitutes. In some cases, the rOVA composition may have a gel strength within the range from 100 g to 1500 g, from 500 g to 1500 g, or from 700 g to 1500 g. In some cases, an rOVA composition has a gel strength of about or at least 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an rOVA composition has a gel strength of up to 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an rOVA composition has a gel strength of about or at least 50%, 55%, 60%, 65%, 70%, 75%, 80%, 85%, 90%, 95%, 100% relative to an egg white, nOVA or egg white substitutes. In some cases, an rOVA composition has a gel strength of up to 50%, 55%, 60%, 65%, 70%, 75%, 80%, 85%, 90%, 95%, 100% relative to an egg white, nOVA or egg white substitutes.

rOVA compositions disclosed herein can provide structure, texture or a combination of structure and texture. In some embodiments, rOVA is added to a food ingredient or food product for baking and the rOVA provides structure, texture or a combination of structure and texture to the baked product. rOVA can be used in such baked products in place of native egg white, native egg or native egg protein. The addition of rOVA to baked products can also provide protein fortification to improve the nutritional content. In some embodiments, rOVA is used in a baked product in an amount between 0.1% and 25% on a weight/weight or weight/volume basis. In some embodiments, rOVA is used in a baked product in an amount between 0.1% and 5%. In some cases, rOVA provides the structure and/or texture of egg-white in absence of any other egg-white proteins.

In some embodiments, the final product containing the protein of interest such as rOVA comprises one or more characteristics described in Table 10. For example, in some embodiments, the final product recovered from the process of the present methods can comprise a moisture content ranging from 5 to 10% w/w. In some embodiments, the final product recovered from the process of the present methods has microbe levels less than 5000 CFU/g, less than 4000 CFU/g, less than 3000 CFU/g, less than 2000 CFU/g, less than 1000 CFU/g, less than 500 CFU/g, less than 200 CFU/g, less than 100 CFU/g, or less than 50 CFU/g. In some embodiments, the final protein product recovered from the process of the present methods is free of Salmonella and/or E. coli. In some embodiments, the final protein product recovered from the process of the present methods has a reduced amount of heavy metal levels compared to a final protein produced recovered using a different process. In some embodiments, the heavy metal level of the final protein product is <0.5 ppm, <0.4 ppm, <0.3 ppm, <0.2 ppm, or <0.1 ppm. In some embodiments, the level of mercury of the final protein product is <0.5 ppm, <0.4 ppm, <0.3 ppm, <0.2 ppm, or <0.1 ppm. In some embodiments, the level of Arsenic of the final protein product is <0.5 ppm, <0.4 ppm, <0.3 ppm, <0.2 ppm, or <0.1 ppm. In some embodiments, the level of Lead of the final protein product is <0.5 ppm, <0.4 ppm, <0.3 ppm, <0.2 ppm, or <0.1 ppm. In some embodiments, the level of Cadmium of the final protein product is <0.5 ppm, <0.4 ppm, <0.3 ppm, <0.2 ppm, or <0.1 ppm.

In some embodiments, the final product comprises a foam capacity, foam stability, conductivity, and/or pH recited in Table 11.

rOVA compositions disclosed herein can be compatible with gluten formation, such that the rOVA can be used where gluten formation provides structure, texture and/or form to a food ingredient or food product. Exemplary baked products in which rOVA can be used as an ingredient include, but are not limited to cake, cookie, bread, bagel, biscuits, muffin, cupcake, scone, pancake, macaroon, choux pastry, meringue, and soufflé. For example, rOVA can be used as an ingredient to make cakes such as pound cake, sponge cake, yellow cake, or angel food cake, where such cakes do not contain any native egg white, native whole egg or native egg protein. Along with rOVA, baked products may contain additional ingredients such as flour, sweetening agents, gum, hydrocolloids, starches, fibers, flavorings (such as flavoring extracts) and other protein sources. In some embodiments, a baked product may include rOVA and at least one fat or oil, at least one grain starch, and optionally at least one sweetener. Grain starch for use in such compositions include flours such as wheat flour, rice flour, corn flour, millet flour, spelt flour, and oat flour, and starches such as from corn, potato, sorghum, and arrowroot. Oil and fat for use in such compositions include plant-derived oils and fats, such as olive oil, corn oil, avocado oil, nut oils (e.g., almond, walnut and peanut) and safflower oil. rOVA may provide such baked goods with at least one characteristic of an egg white such as binding, springiness, aeration, browning, texturizing, humectant, and cohesiveness of the baked product. In some cases, the baked product does not comprise any natural egg white or natural egg, and/or does not include any other egg white derived proteins except rOVA. In some cases, rOVA is provided to the baked composition as an ingredient, such as starting with a concentrate, isolate or powder form of rOVA. In some cases, the rOVA provided as an ingredient for baked products is at a pH range between about 3.5 and 7.0. In some cases, a sweetener is included in the baked product such as a sugar, syrup, honey or sugar-substitute.

rOVA compositions disclosed herein can also be used to prepare egg-less food products, such as food products made where native whole egg or native egg white is a primary or featured ingredient such as scramble, omelet, patty, soufflé, quiche and frittata. In some embodiments, rOVA provides one or more functional features to the preparation including foaming, coagulation, binding, structure, texture, film-formation, nutritional profile, absence of cholesterol (i.e., cholesterol free) and protein fortification. Such egg-less preparations can be vegan, vegetarian, halal, or kosher, or a combination thereof. An egg-less preparation (also referred to as an egg-white substitute) may include rOVA and at least one fat or oil, a polysaccharide or polysaccharide-containing ingredient, and a starch. In some cases, the egg-less preparation may also include a flavoring agent (such as to provide a salty, sulfur-like or umami flavor), and/or a coloring agent (for example to provide yellow-like or off-white color to the baked product). In some cases, the inclusion or rOVA in the egg-less preparation provides a characteristic of natural (native) egg white such as hardness, adhesiveness, fracturability, cohesiveness, gumminess and chewiness when the composition is heated or cooked. Exemplary polysaccharide or polysaccharide-containing ingredients for such compositions include gellan gum, sodium alginate, and psyllium. Oil and fat for use in such compositions include plant-derived oils and fats, such as olive oil, corn oil, avocado oil, and safflower oil.

rOVA compositions disclosed herein can be used for a processed meat product or meat-like product, or for fish-like or shell-fish-like products. In such products, rOVA can provide one or more functional characteristics such as protein content and protein supplementations as well as binding, texturizing properties. Exemplary meat and meat-like products include burger, patty, sausage, hot dog, sliced deli meat, jerky, bacon, nugget and ground meat-like mixtures. Meat-like products can resemble beef, pork, chicken, lamb and other edible and consumed meats for humans and for other animals. Fish-like and shell-fish like products can resemble, for example, fish cakes, crab cakes, shrimp, shrimp balls, fish sticks, seafood meat, crab meat, fish fillets and clam strips. In some embodiments, rOVA is present in an amount between about 0.1% and 30% w/w/or w/v in the meat or meat-like product. In some embodiments, rOVA is used for a meat-like product (also referred to as a meat-analog and includes at least one fat or oil; and a plant-derived protein. Oil and fat for use in such compositions include plant-derived oils and fats, such as olive oil, corn oil, avocado oil, and safflower oil. Plant-derived proteins for use in meat analogs include soy protein, nut proteins, pea protein, lentil and other pulse proteins and whey proteins. In some cases, such plant protein is extruded, in other cases, such plant protein is non-extruded protein. In some cases, a meat analog includes rOVA at about 2% to 15% (w/w). In some cases, for meat analog compositions, rOVA acts as a binding agent, a gelling agent or a combination of a binding and gelling agent for such compositions.

rOVA compositions disclosed herein can be employed in coatings for food products. For example, rOVA can provide binding or adhesion characteristics to adhere batter or breading to another food ingredient. rOVA can be used as an “egg-less egg wash” where the rOVA protein provides appearance, color and texture when coated onto other food ingredients or food products, such as baked products. In one example, the “egg-less egg wash” may be used to coat a baked good such that the baked good adheres to a coating (e.g., seed, salt, spice, and herb). The addition of rOVA as a coating to a food product can provide a crunchy texture or increase the hardness, for example, of the exterior of a food product such as when the product is cooked, baked or fried.

rOVA compositions disclosed herein include sauces and dressings, such as an eggless mayonnaise, commercial mayonnaise substitutes, gravy, sandwich spread, salad dressing or food sauce. Inclusion of rOVA in a sauce or dressing, and the like, can provide one or more characteristics such as binding, emulsifying, odor neutrality, and mouthfeel. In some embodiments rOVA is present in such sauces and dressing in an amount between 0.1% and 3% or between about 3% and about 5% w/w/or w/v. In some cases, the amount of rOVA in a sauce or dressing may be substantially similar to the amount of whole egg, egg-white or nOVA used in a commercially available or commonly used recipe. Exemplary sauces and dressing include mayonnaise, commercial mayonnaise substitutes, alfredo sauce, and hollandaise sauce. In some embodiments, the rOVA-containing sauce or dressing does not contain whole egg, egg white, or any other protein extracted from egg. In some cases, the sauce, dressing or other emulsified product made with rOVA includes at least one fat or oil and water. Exemplary fats and oils for such compositions include corn oil, safflower oil, nut oils, and avocado oil.

rOVA compositions can be used to prepare confectionaries such as eggless, animal-free, vegetarian and vegan confectionaries. rOVA can provide one or more functional features to the confectionary including odor neutrality, flavor, mouthfeel, texture, gelling, cohesiveness, foaming, frothiness, nutritional value and protein fortification. In some embodiments, the prepared confectionary containing rOVA does not contain any native egg protein or native egg white. rOVA in such confectionaries can provide a firm or chewy texture. In some embodiments, rOVA is present between about 0.1% and 15% in a confectionary. Exemplary confectionaries include a gummy, a taffy, a divinity candy, meringue, marshmallow, and a nougat. In some embodiments, a confectionary includes rOVA, at least one sweetener and optionally a consumable liquid. Exemplary sweeteners include sugar, honey, sugar-substitutes and plant-derived syrups. In some cases, the rOVA is provided as an ingredient for making confectionaries at a pH between about 3.5 and about 7. In some cases, the rOVA is present in the confectionary composition at about 2% to about 15% (w/v). In some embodiments, the confectionary is a food product such as a meringue, a whipped dessert, or a whipped topping. In some embodiments, rOVA in the confectionary provides foaming, whipping, fluffing or aeration to the food product, and/or provides gelation. In some cases, the confectionary is a liquid, such as a foamed drink. In some cases, the liquid may include a consumable alcohol (such as in a sweetened cocktail or after-dinner drink).

rOVA compositions herein can be used in dairy products, dairy-like products or dairy containing products. For example, rOVA can be used in preparations of beverages such as a smoothie, milkshake, “egg-nog”, and coffee beverage. In some embodiments, rOVA is added to additional ingredients where at least one ingredient is a dairy ingredient or dairy-derived ingredient (such as milk, cream, whey, and butter). In some embodiments, rOVA is added to additional ingredients to create a beverage that does not contain any native egg protein, native egg white or native egg. In some embodiments, rOVA is an ingredient in a beverage that does not contain any animal-derived ingredients, such as one that does not contain any native egg-derived or any dairy-derived ingredients. Examples of such non-dairy derived drinks include nut milks, such as soy milk or almond milk. rOVA can also be used to create beverage additions, such as creamer or “milk” to provide protein, flavor, texture and mouthfeel to a beverage such as a coffee, tea, alcohol-based beverages or cocoa. In some embodiments, rOVA is present in a beverage ingredient or beverage addition in an amount between about 0.1% and 20% w/w or w/v.

In some embodiments herein, rOVA can be used to prepare a dairy-like product such as yogurt, cheese or butter. Dairy products with rOVA can include other animal-based dairy components or proteins. In some embodiments, dairy products prepared with rOVA do not include any animal-based ingredients.

Preparations of dessert products can be prepared using rOVA. In dessert products rOVA can provide one or more characteristics such as creamy texture, low fat content, odor neutrality, flavor, mouthfeel, texture, binding, and nutritional value. rOVA may be present in an ingredient or set of ingredients that is used to prepare a dessert product. Exemplary dessert products suitable for preparation with rOVA include a mousse, a cheesecake, a custard, a pudding, a popsicle and an ice cream. In some embodiments, dessert products prepared to include rOVA are vegan, vegetarian or dairy-free. Dessert products that include rOVA can have an amount of rOVA that is between about 0.1% and about 10% rOVA w/w or w/v.

rOVA can be used to prepare a snack food, such as a protein bar, an energy bar, a nutrition bar or a granola bar. The rOVA can provide characteristics to the snack food including one or more of binding, protein supplementation, flavor neutrality, odor neutrality, coating and mouth feel. In some embodiments, rOVA is added to a preparation of a snack food in an amount between about 0.1% and 30% w/w or w/v.

rOVA can be used for nutritional supplements such as in parenteral nutrition, protein drink supplements, protein shakes where rOVA provides a high protein supplement. In some embodiments, rOVA can be added to such compositions in an amount between about 10% and 30% w/w or w/v.

In some embodiments, rOVA compositions can be used as an egg-replacer and an egg white-replacer. rOVA can be mixed or combined with at least one additional component to form the egg white replacer. rOVA can provide one or more characteristics to the egg-replacer or egg white-replacer, such as gelling, foaming, whipping, fluffing, binding, springiness, aeration, creaminess and cohesiveness. In some embodiments, characteristic is the same or better than a native egg or native egg white provided in the same amount or concentration (w/w or w/v). In some embodiments, the egg-replacer or egg white-replacer, does not contain any egg, egg white, protein extracted or isolated from egg.

The rOVA-containing food ingredient and food products, such as described herein, can contain additional ingredients or components. For example, rOVA compositions can be prepared with an additional component such as one or more of a sweetener, a gum, a flavoring, a thickener, an acidulant and an emulsifier. Other ingredients such as flour, grains, oils and fats, fiber, fruit and vegetables can be combined with rOVA. Such rOVA compositions can be vegan, vegetarian, halal, kosher and animal-free, or a combination thereof. In some embodiments, rOVA can be a food ingredient or prepared for a food product that is normally animal based or normally contains animal-derived components, such as meat, dairy or eggs.

Compositions including rOVA including food ingredients and food products can be compatible with one or more steps of consumables preparation such as heated, baked, grilled, roasted, braised, microwaved, broiled, boiled, steamed, extruded, deep fried, or pan-fried, or processed using ohmic heating, Sue Vide, freezing, chilling, blanching, packaging, canning, bleaching, enriching, drying, pressing, grinding, mixing, par cooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, smoking, curing, salting, preserving, pickling, fermenting, homogenizing, pasteurizing, sterilizing, irradiating, cold plasma processing, high pressure processing, pulse electric field processing, microwave assisted thermal sterilization, stabilizing, blending, pureeing, fortifying, refining, hydrogenating, aging, extending shelf life, or adding enzymes.

Food ingredients and food products prepared with rOVA can be essentially free of any microbial cells or microbial cell debris. For instance, rOVA may be secreted from a microbial host cell and isolated from microbial cells, culture media and/or microbial cell debris.

In some embodiments, rOVA may be prepared as a whole cell extract or fractionated extract such that an rOVA composition contains microbial cells and/or microbial cell components.

In one embodiment, an rOVA composition is prepared for animal consumption where the rOVA is present in a whole cell extract or fractionated extract such that an rOVA composition contains microbial cells and/or microbial cell components. In some embodiments, an rOVA composition is prepared for animal consumption where rOVA is isolated from microbial cells, culture media and microbial cell debris. Exemplary compositions for animal consumption can include a pet food, an animal feed, a chewy treat, bone broth, smoothie or other liquid for animal nutrition and a solid nutritional supplement suitable for animal consumption. In these cases, the microbial cell extract or microbial cell debris may provide additional nutritional value.

Animals which may consume rOVA compositions can include companion animals (e.g., dog, cat, horse), farm animals, exotic animals (lion, tiger, zebra) as well as livestock (such as cow, pig, sheep, goat). rOVA compositions as described herein can also be used for aquaculture (such as for fish and shellfish) and for avian nutrition (such as for bird pets, zoo birds, wild birds, fowl and birds raised for human and animal food).

In some embodiments of the consumable food compositions described herein, the composition is essentially free of animal-derived components, whey protein, caseinate, fat, lactose, hydrolyzed lactose, soy protein, collagen, hydrolyzed collagen, or gelatin, or any combination thereof. A composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise and commercial mayonnaise substitutes) and such composition may include up to about 60% fat or a reduced-fat composition (e.g., reduced fat mayonnaise and commercial mayonnaise substitutes) and such composition may include lesser percentages of fat. A composition that free of an animal-derived component can be considered vegetarian and/or vegan.

In some embodiments, an rOVA powder composition comprises less than 5% ash. The term “ash” is an art-known term and represents inorganics such as one or more ions, elements, minerals, and/or compounds. In some cases, the rOVA powder composition comprises less than 5%, 4.5%, 4%, 3.5%, 3%, 2.5%, 2%, 1.5%, 1%, 0.75%, 0.5%, 0.25% or 0.1% ash weight per total weight (w/w) and/or weight per total volume (w/v).

In some embodiments, the moisture content of an rOVA powder composition may be less than 15%. The rOVA powder composition may have less than 15%, 12%, 10%, 8%, 6%, 5%, 3%, 2% or 1% moisture weight per total weight (w/w) and/or weight per total volume (w/v). In some embodiments, the carbohydrate content of an rOVA powder composition may be less than 30%. The rOVA powder composition may have less than 30%, 27%, 25%, 22%, 20%, 17%, 15%, 12%, 10%, 8%, 5%, 3% or 1% carbohydrate content w/w or w/v.

### 5.4.1. Sensory Neutrality and Improved Sensory Appeal

In some embodiments, in addition to the egg-white like properties, the addition of rOVA to a consumable food composition provides increased protein nutritional content, sensory neutrality or an improved sensory appeal as compared to other proteins in such compositions. As used herein “sensory neutrality” refers to the absence of a strong or distinctive taste, odor (smell) or combination of taste and smell, as well as texture, mouth-feel, aftertaste and color. A sensory panel such as one described in Kemp et al. 2009 may be used by a trained sensory analyst. Sensory neutrality may provide an improved sensory appeal to a taster, such as a tester of foods or a consumer, when a consumable food composition containing rOVA is compared with another like composition that has a different protein such as nOVA, whey protein, pea protein, soy protein, whole egg or egg white protein at the same concentration.

In some embodiments, rOVA when added to a consumable food composition is substantially odorless, such as measured by a trained sensory analyst, in comparison with different solutions/products with a different protein component present in an equal concentration to the rOVA containing solution/product, for example, in the comparison is whey, soy, collagen, pea, egg white solid isolates and/or nOVA. In some embodiments of the rOVA compositions described herein, such compositions are essentially odorless at a protein concentration between about 0.5-1%, 1%-5%, 5-10%, 10-15%, 15-20%, 20-25%, 25-30% rOVA weight per total weight (w/w) and/or weight per total volume (w/v) or at a protein concentration of about 0.1, 1, 5, 10, 11, 12, 13, 14, 15, 16, 17, 18, 19, 20, 21, 22, 23, 24, 25, 26, 27, 28, 29 or 30 g of total rOVA protein per 100 mL solution (e.g., per 100 mL

In some embodiments, the addition of rOVA to a consumable food composition also provides a neutral taste in addition to the characteristics such as egg-white like properties and increased protein nutrition content. A neutral taste can be measured for example, by a trained sensory analyst in comparison with solutions containing a different protein present in an equal concentration to the rOVA, for example, whey, soy, collagen, pea, whole egg, and egg white solid isolates (including native OVA).

In some embodiments, the addition of rOVA provides a reduction in a certain odor and/or taste that is associated with other proteins or egg-whites. For example, addition of rOVA has less of an “egg-like” odor or taste as compared to the addition of whole egg, fractionated egg or egg-white to a consumable food composition. In some embodiments, addition of rOVA has less of a metallic odor or taste as compared to other protein sources.

In some embodiments, the addition of rOVA has an improved mouth-feel as compared to the addition of other protein sources used to produce egg-white like properties. For example, the addition of rOVA is less grainy or has less precipitates or solids as compared to other protein sources.

In some embodiments, the addition of rOVA has an improved texture, for example, as compared to other available supplemental protein sources.

A consumable composition with rOVA may also have an improved sensory appeal as compared to the composition without rOVA or with a different protein present in an equal concentration to the rOVA. Such improved sensory appeal may relate to taste and/or smell. Taste and smell can be measured, for example, by a trained sensory analyst. In some instances, a sensory analyst compares a consumable composition with rOVA to one without it or with a different protein or protein source in an equivalent amount.

As described herein, a consumable composition herein can be in a liquid form. A liquid form can be an intermediate product such as soluble rOVA solution. In some cases, a liquid form can be a final product, such as a beverage comprising rOVA. Example of different types of beverages contemplated herein include: a juice, a soda, a soft drink, a flavored water, a protein water, a fortified water, a carbonated water, a nutritional drink, an energy drink, a sports drink, a recovery drink, an alcohol-based drink, a heated drink, a coffee-based drink, a tea-based drink, a plant-based milk, a nut milk, a milk based drink, a non-dairy, plant based mild drink, infant formula drink, and a meal replacement drink.

### 5.4.1.1 pH of Compositions

In some embodiments, the pH of an rOVA composition ranges from 3 to 8 (e.g., 3, 3.25, 3.5, 4, 4.25, 4.5, 5, 5.25, 5.5, 6, 6.25, 6.5, 7, 7.25, 7.5, or 8). In some embodiments, the pH of an rOVA composition may be 3.5 to 8. The pH of an rOVA composition may be at least 3.5. The pH of an rOVA composition may be at most 8. The pH of an rOVA composition may be 3.5 to 4, 3.5 to 4.5, 3.5 to 5, 3.5 to 5.5, 3.5 to 6, 3.5 to 6.5, 3.5 to 7, 3.5 to 7.5, 3.5 to 8, 4 to 4.5, 4 to 5, 4 to 5.5, 4 to 6, 4 to 6.5, 4 to 7, 4 to 7.5, 4 to 8, 4.5 to 5, 4.5 to 5.5, 4.5 to 6, 4.5 to 6.5, 4.5 to 7, 4.5 to 7.5, 4.5 to 8, 5 to 5.5, 5 to 6, 5 to 6.5, 5 to 7, 5 to 7.5, 5 to 8, 5.5 to 6, 5.5 to 6.5, 5.5 to 7, 5.5 to 7.5, 5.5 to 8, 6 to 6.5, 6 to 7, 6 to 7.5, 6 to 8, 6.5 to 7, 6.5 to 7.5, 6.5 to 8, 7 to 7.5, 7 to 8, or 7.5 to 8. The pH of an rOVA composition may be 3.5, 4, 4.5, 5, 5.5, 6, 6.5, 7, 7.5, or 8. An rOVA composition with a pH between 3.5 to 7 may have one or more improved functionalities as compared to nOVA, egg white or egg-white substitute compositions.

The pH of an rOVA composition may be 2 to 3.5. The pH of an rOVA composition may be at least 2. The pH of an rOVA composition may be at most 3.5. The pH of an rOVA composition may be 2 to 2.5, 2 to 3, 2 to 3.5, 2.5 to 3, 2.5 to 3.5, or 3 to 3.5. The pH of an rOVA composition may be 2, 2.5, 3, or 3.5.

The pH of an rOVA composition may be 7 to 12. The pH of an rOVA composition may be at least 7. The pH of an rOVA composition may be at most 12. The pH of an rOVA composition may be 7 to 7.5, 7 to 8, 7 to 8.5, 7 to 9, 7 to 9.5, 7 to 10, 7 to 10.5, 7 to 11, 7 to 11.5, 7 to 12, 7.5 to 8, 7.5 to 8.5, 7.5 to 9, 7.5 to 9.5, 7.5 to 10, 7.5 to 10.5, 7.5 to 11, 7.5 to 11.5, 7.5 to 12, 8 to 8.5, 8 to 9, 8 to 9.5, 8 to 10, 8 to 10.5, 8 to 11, 8 to 11.5, 8 to 12, 8.5 to 9, 8.5 to 9.5, 8.5 to 10, 8.5 to 10.5, 8.5 to 11, 8.5 to 11.5, 8.5 to 12, 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. The pH of an rOVA composition may be 7, 7.5, 8, 8.5, 9, 9.5, 10, 10.5, 11, 11.5, or 12.

In some embodiments, the pH of rOVA may be adjusted prior to its inclusion in a composition or its use as an ingredient. In some embodiments, the pH of rOVA is adjusted during the purification and/or isolation processes. In some embodiments, the pH of the rOVA for use in an ingredient or in production of a food product composition is adjusted to between about 3.5 to about 7.0. In some cases, the pH of rOVA may be adjusted to more than one pH during the production process. For example, rOVA may be expressed in a host cell such as a a microbial cell, and in some cases the rOVA is secreted by the host cell into the growth media (e.g., liquid media). rOVA is separated from the host cells and such separation step may be performed at a selected pH, for example at a pH of about 3.5. In some cases, the rOVA at such separation pH may not be soluble or may not be fully soluble and the pH is adjusted to a higher pH, such as about pH 12. The rOVA may then be adjusted to a final pH between about 3.5 and about 7.0. Separation of rOVA from other components of the host cells or other components of the liquid media can include one or more of ion exchange chromatography, such as cation exchange chromatography and/or anion exchange chromatography, filtration and ammonium sulfate precipitation.

### 5.4.1.2 Additional Components of Compositions

The consumable food compositions containing rOVA disclosed herein and the methods of making such compositions may including adding or mixing the rOVA with one or more ingredients. For example, food additives may be added in or mixed with the compositions. Food additives can add volume and/or mass to a composition. A food additive may improve functional performance and/or physical characteristics. For example, a food additive may prevent gelation or increased viscosity due to the lipid portion of the lipoproteins in the freeze-thaw cycle. An anticaking agent may be added to make a free-flowing composition. Carbohydrates can be added to increase resistance to heat damage, e.g., less protein denaturation during drying and improve stability and flowability of dried compositions. Food additives include, but are not limited to, food coloring, pH adjuster, natural flavoring, artificial flavoring, flavor enhancer, batch marker, food acid, filler, anticaking agent (e.g., sodium silico aluminate), antigreening agent (e.g., citric acid), food stabilizer, foam stabilizer or binding agent, antioxidant, acidity regulatory, bulking agent, color retention agent, whipping agent (e.g., ester-type whipping agent, triethyl citrate, sodium lauryl sulfate), emulsifier (e.g., lecithin), humectant, thickener, excipient, solid diluent, salts, nutrient, sweetener, glazing agent, preservative, vitamin, dietary elements, carbohydrates, polyol, gums, starches, flour, oil, or bran.

Food coloring includes, but is not limited to, FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), anthocyanins, annatto, betanin, butterfly pea, caramel coloring, chlorophyllin, elderberry juice, lycopene, carmine, pandan, paprika, turmeric, curcuminoids, quinoline yellow, carmoisine, Ponceau 4R, Patent Blue V, and Green S.

Ingredients for pH adjustment include, but are not limited to, Tris buffer, potassium phosphate, sodium hydroxide, potassium hydroxide, citric acid, sodium citrate, sodium bicarbonate, and hydrochloric acid.

Salts include, but are not limited, to acid salts, alkali salts, organic salts, inorganic salts, phosphates, chloride salts, sodium salts, sodium chloride, potassium salts, potassium chloride, magnesium salts, magnesium chloride, magnesium perchlorate, calcium salts, calcium chloride, ammonium chloride, iron salts, iron chlorides, zinc salts, and zinc chloride.

Nutrient includes, but is not limited to, macronutrient, micronutrient, essential nutrient, non-essential nutrient, dietary fiber, amino acid, essential fatty acids, omega-3 fatty acids, and conjugated linoleic acid.

Sweeteners include, but are not limited to, sugar substitute, artificial sweetener, acesulfame potassium, advantame, alitame, aspartame, sodium cyclamate, dulcin, glucin, neohesperidin dihydrochalcone, neotame, P-4000, saccharin, aspartame-acesulfame salt, sucralose, brazzein, curculin, glycyrrhizin, glycerol, inulin, mogroside, mabinlin, malto-oligosaccharide, mannitol, miraculin, monatin, monellin, osladin, pentadin, stevia, trilobatin, and thaumatin.

Carbohydrates include, but are not limited to, sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), sialic acid, monosaccharides, disaccharides, polysaccharides (e.g., polydextrose, maltodextrin), and starch.

Polyols include, but are not limited to, xylitol, maltitol, erythritol, sorbitol, threitol, arabitol, hydrogenated starch hydrolysates, isomalt, lactitol, mannitol, and galactitol (dulcitol).

Gums include, but are not limited to, gum arabic, gellan gum, guar gum, locust bean gum, acacia gum, cellulose gum, and xanthan gum.

Vitamins include, but are not limited to, niacin, riboflavin, pantothenic acid, thiamine, folic acid, vitamin A, vitamin B6, vitamin B12, vitamin D, vitamin E, lutein, zeaxanthin, choline, inositol, and biotin.

Dietary elements include, but are not limited to, calcium, iron, magnesium, phosphorus, potassium, sodium, zinc, copper, manganese, selenium, chlorine, iodine, sulfur, cobalt, molybdenum, nickel, and bromine.

### 6. EXAMPLES

### 6.1. Example 1: Methods for Recovering and Purifying an Illustrative Protein of Interest

FIG. 1A is a flow chart that illustrates illustrative steps of methods of the present disclosure. FIG. 1B illustrates procedures of corresponding steps in FIG. 1A.

The steps of the illustrated method include: obtaining recombinant fungal cells capable of expressing a secreted protein of interest; culturing the recombinant fungal cells under conditions that promote expression and secretion of the recombinant protein of interest into a culturing medium; centrifuging the culturing medium to remove the recombinant fungal cells and other cellular components; without microfiltering the centrifuged culturing medium to further remove any residual cell components prior to introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

Referring to FIG. 1B, the culture medium was centrifuged to remove recombinant fungal cells and other cellular components. Ammonium sulfate (200 g/l; 65% w/v) was added to the supernatant containing the protein of interest released from the recombinant fungal cells. The precipitated protein of interest was resuspended to remove excess salt via diafiltering and/or ultrafiltering the solubilized protein of interest, which was then diluted to a concentration of 50 g/L. The protein of interest was further microfiltered (0.2 μm) to improve clarification, followed by drying (e.g., spray drying) to produce a dried protein product.

In some cases, one or more of the above steps are performed one or more times. As shown in FIG. 1C, the method comprises obtaining recombinant fungal cells capable of expressing a secreted protein of interest; culturing the recombinant fungal cells under conditions that promote expression and secretion of the recombinant protein of interest into a culturing medium; centrifuging the culturing medium to remove the recombinant fungal cells and other cellular components for at least two times; introducing ammonium sulfate to the culturing medium to achieve an ammonium sulfate concentration above 200 g/l, thereby precipitating the secreted protein of interest; recovering the precipitated protein of interest; solubilizing the precipitated secreted protein of interest with water to obtain a solubilized protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below the isoelectric point (pI) of the protein of interest and introducing ammonium sulfate.

In some cases, one more of the above steps is omitted. For example, the method may conclude when the protein of interest is precipitated and recovered. In this case, the method comprises steps of: obtaining recombinant fungal cells capable of expressing a secreted protein of interest; culturing the recombinant fungal cells under conditions that promote expression and secretion of the recombinant protein of interest into a culturing medium; introducing ammonium sulfate to the culturing medium to achieve an ammonium sulfate concentration above 200 g/l, thereby precipitating the secreted protein of interest; and recovering the precipitated protein of interest.

As in FIG. 1D, the method comprises steps of: obtaining recombinant fungal cells capable of expressing a secreted protein of interest; culturing the recombinant fungal cells under conditions that promote expression and secretion of the recombinant protein of interest into a culturing medium; introducing ammonium sulfate to the culturing medium to achieve an ammonium sulfate concentration above 200 g/l, thereby precipitating the secreted protein of interest; and recovering the precipitated protein of interest; diafiltering and/or ultrafiltering the solubilized protein of interest; microfiltering the diafiltered and/or ultrafiltered protein of interested; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

As in FIG. 1E, the method comprises steps of: obtaining recombinant fungal cells capable of expressing a secreted protein of interest; culturing the recombinant fungal cells under conditions that promote expression and secretion of the recombinant protein of interest into a culturing medium; microfiltering the centrifuged culturing medium to further remove any residual cell components; ultrafiltering the solubilized protein of interest; centrifuging the culturing medium to remove the recombinant fungal cells and other cellular components; diafiltered the protein of interested; centrifuging the culturing medium to remove the recombinant fungal cells and other cellular components; testing purity of the protein of interest with chromatography; ultrafiltering the solubilized protein of interest; microfiltering the centrifuged culturing medium to further remove any residual cell components; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

As shown in FIG. 1F, the method comprises steps of: obtaining recombinant fungal cells capable of expressing a secreted protein of interest; culturing the recombinant fungal cells under conditions that promote expression and secretion of the recombinant protein of interest into a culturing medium; microfiltering the centrifuged culturing medium to further remove any residual cell components; introducing ammonium sulfate to the culturing medium to achieve an ammonium sulfate concentration above 200 g/l, thereby precipitating the secreted protein of interest; and recovering the precipitated protein of interest; diafiltering and/or ultrafiltering the solubilized protein of interest; microfiltering the diafiltered and/or ultrafiltered protein of interested; microfiltering the centrifuged culturing medium to further remove any residual cell components; and drying the further microfiltered protein of interest, thereby obtaining a dried protein product. The method does not require adding an acid to the microfiltered culturing medium to reduce the pH to about or below pH 4.5 or the isoelectric point (pI) of the protein of interest and introducing ammonium sulfate.

### 6.2. Example 2: Analysis of Various Parameters on Recovery and Purity of Protein of Interest

The effect of various parameters including pH, salt concentration, purification with chromatography, ultrafiltration, microfiltration, and combinations were tested. The results were provided in Table 2 below. Here, the illustrative protein was recombinant ovalbumin (rOVA).

TABLE 2
Analysis of various parameters on recovery
and purity of protein of interest

| Expt Name | Experiment Goal | Outcome |
|---|---|---|
| NES-001 | pH Precipitation | pH 3.25 & 3.5 performed |
| | Exploration: | best, with only 17.5 & |
| | pH 3.25-5.5 (protein | 17.3% POI remaining in |
| | fractionation) | supernatant |
| NES-002 | Salt precipitation (SPR) | 400 g/L Ammonium Sulfate |
| | Exploration: 100-400 g/L | performed best, 84% POI in |
| | Ammonium Sulfate | pellet and 16% in supernatant. |
| | tested | |
| NES-003 | Chromatography | CEX was deemed to be not a |
| | (CEX) tested, Pichia | viable separation method and |
| | OVA method (for | the protein impurity levels |
| | protein fractionation) | did not warrant the use of |
| | | this unit option. |
| NES-004 | V1 process: Salt | Low recoveries, under 30% |
| | precipitation(SPR) −> | overall, needs to be validated |
| | Ultrafiltration (UF1) −> | and optimized |
| | Microfiltration | |
| | (MF2, MF3) | |
| NES-005 | V1 process: | 53.1% overall recovery, QA |
| | SPR > UF1 > MF2 > MF3 | results showed improved foam |
| | | stability and capacity over |
| | | typical r-OVA |
| NES-006 | V1 process: | Discovered and generated a |
| (007 | SPR > UF1 > MF2 > MF3 | mass balance for the non- |
| validates) | | protein impurities being |
| | | produced, largely removed |
| | | in SPR step. 75% POI |
| | | recovery after MF2 |
| | | (NES-006) |

As shown, adding acid to lower the pH to pH 3.25 and pH 3.5 yield best results but with only 17.5% and 17.3%, respectively, protein of interest (POI) remaining in supernatant (NES-001). Ammonium sulfate at 400 g/L performed best, yielding 84% protein of interest in pellet and 16% in supernatant (NES-002). Chromatography (CEX) was tested in Pichia OVA method for protein fractionation. CEX was deemed to be not a viable separation method and the protein impurity levels did not warrant the use of this unit option (NES-003). Combining salt precipitation “Salt ppt” (e.g., addition of ammonium sulfate) with ultrafiltration and microfiltration yielded low recovers, giving under 30% of protein of interest overall (NES-004). However, combining, in sequence, salt precipitation, ultrafiltration, and twice microfiltration yielded 53.1% of protein of interest overall recovery. A food composition made of the product showed improved foam stability and capacity over typical r-OVA (NES-005). Further testing of the same sequence in NES-005 discovered and generated a mass balance for the non-protein impurities being produced, largely removed in SPR step. About 75% protein of interest were recovered after second microfiltration (NES-006).

### 6.3. Example 3: Analysis of Ultrafiltration Effects on Recovery and Purity of Protein of Interest

In this example, ultrafiltration parameters were tested for membrane cutoff, manufacturers, and diafiltration buffers. The results are summarized in Table 3 below.

TABLE 3
Analysis ultrafiltration effects on recovery
and purity of protein of interest

| Expt Name | Experiment Goal | Outcome |
|---|---|---|
| NES-009 | Test w/o SPR | 33% MF2 recovery, UF1 |
| | (UF1 > MF2 > MF3) | removes less non protein |
| | | impurities than SPR |
| NES-010 | Test w/o SPR, and salt | 43% overall Recovery |
| | buffer diafiltration | |
| | (UF1 w/NaCl DF −> | |
| | MF2) | |
| NES-011 | Test w/o SPR, salt | Lower UF recovery, |
| | buffer diafiltration and | 50% overall |
| | MF alternative (UF1 | |
| | w/NaCl DF > Past) | |
| NES-012 | Test membrane | 36% overall POI |
| | chemistry -Koch | recovery |
| | membranes in v1 | |
| | process | |
| | (SPR > UF1 > MF2) | |

Referring to Table 3, NES-009 is a modification of NES-006, it tested recovery of protein of interest (POI) released from the recombinant fungal cells without ammonium sulfate precipitation (SPR). About 33% were recovered by microfiltration. Ultrafiltration (UF1) removed less non protein impurities than SPR. NES-010 is a modification of NES-009. It tested processing without ammonium sulfate precipitation (SPR) and sodium chloride diafiltration, followed by microfiltration, yielded about 43% overall recovery. NES-011 is similar to NES-010 but substituted microfiltration with pasteurization. The method yielded lower ultrafiltration recovery, and a final about 50% overall protein of interest. NES-012 is a modification of NES-006, it tested membrane effect on recovery. After salt precipitation, Koch membrane filtrations yielded about 36% overall POI recovery.

### 6.4. Example 4: Validation of Downstream Recovery Processes for Protein of Interest

This example validated VI process (NES-006) as described in Example 1 at an independent facility and tested with larger volumes of broth. Referring to NES-016, approximately, 14.5 L of medium containing the protein of interest released from the recombinant fungal cells were precipitated with ammonium sulfate precipitation (SPR), followed by ultrafiltration and microfiltration. The process yielded about 85% overall POI recovery.

### 6.5. Example 5: Analysis of Host Cell on Recovery and Purity of Protein of Interest

The effect of host cell (fungal strains) in recovery and purity of a protein of interest was assayed. Here, the illustrative protein was recombinant ovalbumin (rOVA). In these experiments, The salt concentrations tested were 100 g/1, 200 g/1, 300 g/l, and 400 g/l. The precipitation steps performed were similar to those of Example 1. The results are provided in Table 4 below.

TABLE 4
Strain testing

| Expt Name | Experiment Goal | Outcome |
|---|---|---|
| NES-017 | Glycosylated OVA | Overall Recovery: 40% which |
| (SP100) | purified using | was higher than expected |
| | chromatography | given the number of steps. |
| | (MF1 > UF1 > CF > DF1 > | |
| | CF2 > CEX > UF2 > MF2) | |
| NES-018 | pH and Salt precipitation | pH not viable, SPR at pH 6 |
| (SP008) | exploration with non- | showed promise, better than |
| | glycosylated OVA | pH 4.5 |
| NES-019 | CF > SPR > UF1 > MF2 | Overall POI Recovery: 78.1% |
| (SP008) | | |
| NES-020 | pH and Salt precipitation | pH not viable, SPR at pH 6 |
| (SP100) | exploration | showed promise, better than |
| | | pH 4.5 |
| NES-021 | CF > SPR > UF1 > MF2 | Overall POI Recovery: 46.7% |
| (SP100) | | |

Referring to Table 4, depending on the host cell (e.g., fungal strain) the rOVA can have a glycosylation different from the wildtype (e.g., natural) OVA. The rOVA can be glycosylated or non-glycosylated. NES-017 tested non-glycosylated OVA purified using chromatography (FIG. 1E). The process yielded about 40% overall POI recovery, which was higher than expected given the number of steps. NES-018 tested pH and salt precipitation (ammonium sulfate precipitation) with glycosylated OVA. The result showed that salt precipitation at pH of 6 unexpectedly yielded better overall POI recovery than at pH to 4.5 or lower. NES-019 showed removal of cell debris by centrifugation and component prior to salt precipitation of glycosylated rOVA and filtrations increased overall POI recovery to about 78.1% (see e.g., FIG. 1A, FIG. 1C). NES-020 showed adding an acid to lower the pH prior to or contemporarily with salt precipitation of non-glycosylated rOVA did not improve overall POI recovery. In fact, SPR at pH 6 performed better than at pH 4.5 or lower. NES-021 tested recovery of non-glycosylated rOVA following the methods illustration in FIG. 1A and FIG. 1C. The process yielded about 56.7% overall POI (non-glycosylated rOVA) recovery.

The results for the various strains and downstream processes are summarized in Table 5 below.

TABLE 5
Results for the various strains and downstream processes

| | Salt precipitation | pH precipitation | Chromatography |
|---|---|---|---|
| Glycosylated | 77% POI recovery | Very low | Not tested |
| (SP008) | (pH 4.5) | recoveries (under | |
| | 91% POI recovery | 10% recovery) | |
| | (pH 6) | | |
| Non- | 55% POI recovery | Very low | 83.2% POI |
| Glycosylated | (pH 4.5) | recoveries | recovery in CEX, |
| (SP100) | 65.6% POI recovery | (4% recovery | 40% overall |
| | (pH 6) | at pH3) | |

Referring to Table 5, for glycosylated strains (e.g., SP008), salt precipitation without lowering pH to 4.5 or lower is effective for recovering OVA with glycosylation. At pH 6, approximately 91% POI was recovered overall. In comparison, at pH4.5, approximately 77% POI was recovered overall. Adding an acid to lower the pH during salt precipitation yielded very low recoveries (under 10% recovery).

For non-glycosylated strains (e.g., SP100), salt precipitation without lowering pH to 4.5 or lower is effective for recovering OVA without glycosylation. At pH6, approximately 65.6% POI was recovered overall. In comparison, at pH4.5, approximately 55% POI was recovered overall. Adding an acid to lower the pH during salt precipitation yielded very low recoveries (under 4% recovery at pH4). Chromatography purification recovered about 83.2% POI, and an overall POI recovery of about 40% in the product.

### 6.6. Example 6: Illustrative Process Scale Up

This example describes a downstream processing of recombinantly-expressed proteins under current food good manufacturing processes (cGMP).

A fungal platform, as disclosed herein, is used to produce an egg white protein through a fermentation process. The protein of interest is secreted extracellularly. The secreted protein of interest undergoes any downstream processing as depicted in FIGS. 1A-1F and a non-limiting example of the process is as follows: Once fermentation is complete, the fermentation broth is diluted, chilled, and clarified using a centrifuge. The solids are disposed of and the centrate is filtered through a 0.2 μm filter (e.g., “microfiltering” in a TFF mode (hollow fiber/spiral wound) to remove any remaining cell debris. The filtrate may be stored at 8° C. for up to 72 hours at this point. Following clarification, in some embodiments, the permeate from the 0.2 μm filtration is reduced in volume by a 10 kDa membrane concentrating by about six to about eight-fold. Ammonium sulfate is then added to the retentate to precipitate the protein of interest. This precipitate is recovered using a centrifuge or Sedicanter®. The precipitate is resuspended to a protein concentration target of 40-50 g/L and the pH adjusted to about 6. This resuspension is dialyzed using a 10 kDa membrane to reduce the conductivity to below 900 μS/cm. Prior to drying the protein product into a powder, a final 0.2 μm filtration is performed as a step to reduce bioburden and then the final product is spray dried. All final product packaging material is suitable for food grade use.

Detailed process description. If material is held for more than four hours without processing, it is kept chilled between 8 and 15° C. to minimize microbial growth.

In the “Precipitation” steps, solid separation occurs via centrifugation; clarification occurs via 0.2 μm Filtration UF+DF, and Precipitation in ammonium sulfate (e.g., 65% w/v). See Table 6.

TABLE 6
Precipitation Parameters

| | |
|---|---|
| Ammonium Sulfate target | 40% w/v |
| concentration | |
| Temperature | 20° C.-25° C. |
| pH | pH 4.5-6.5 |
| Precipitation Time | 4-12 hours |
| Mixing Conditions | Gentle stirring |

The purpose of the ammonium sulfate precipitation step is to further purify the protein. First, ammonium sulfate is added to the retentate from the 10 kDa Ultrafiltration step to create a 40% w/v ammonium sulfate solution.

Proper temperature, pH, salt concentration, and mixing control appear to provide proper formation and growth of the precipitate. Moderate agitation and tank chilling (as compared to using an external heat exchanger and pump) appear to be helpful. Slow addition of the salt with moderate mixing helps allow the salt to fully dissolve and avoids clumps and poor precipitation. When mixed properly, a milky white precipitate will form, whereas hard agitation will cause foaming.

Precipitation continues for about four hours to about twelve depending on mixing and rate of precipitation. Tracking the progress of precipitation with bench centrifugation spin test can demonstrate when the precipitation has stalled out. Further salt or acid additions may be needed to restart precipitation. Monitoring should continue until no further change can be affected or are detected.

The precipitate recovery and dilution are performed according to the parameters provided in Table 7.

TABLE 7
Precipitation Recovery and Resuspension Parameters

| Final Target Protein Concentration | 40-50 g/L |
|---|---|
| Final pH | 6 ± 0.1 |

The precipitate is recovered by use of a centrifuge or a Sedicanter®. Use of a disk stack centrifuge can be challenging due to the physical nature of the protein precipitate. A balance of the feed solids percentage, the feed rate, and the residence time in the bowl helps prevent the solids from adhering to the bowl. In the event solids do adhere, a water rinse through the machine and cyclone is performed and which is not allowed to go out the centrate. This rinse can recover the protein as the POI is very soluble. Typical disk stack centrifuges have wash nozzles on the bowl and cyclone to help remove solids. Notably, the Sedicanter® from Flottweg can provide more efficient recovery of precipitate than a standard centrifuge.

The resultant solid slurry is resuspended back up to the desired volume to ensure complete solubilization. A protein concentration target of about 40 to about 50 g/L will assist proper solubilization; the pH should be increased to about 6 or about 6.5 with sodium hydroxide. This process can continue until no precipitate is seen in suspension.

The suspension is filtered/dialyzed using a 10 kDa Diafiltration and according to the parameters provided in Table 8.

TABLE 8
10 kDa parameters

| | |
|---|---|
| Membrane Type | Polyether sulfone (PES) hollow |
| | fiber/spiral wound (recommended) |
| Membrane size | 10 kDa nominal |
| Temperature | 10° C. ± 5 |
| Final pH | 6.9 ± 0.2 |
| Transmembrane | Up to 2 bar (follow manufacturer |
| pressure (TMP) | recommendation) |
| Concentration | At 50 g/L |
| Average Flux | 5 ± 2 LMH |
| Retentate Conductivity | <1 mS/cm |
| Color of retentate | Clear, golden yellow |

The purpose of the 10 kDa diafiltration is to remove salts from the solution. This filtration is run in a tangential mode. The target protein is in the retentate. The solution at pH 6.5±

0.2 at ˜50 g/L protein concentration is diafiltered until the conductivity in the retentate is <900 μS/cm. The final dialyzed material should be golden yellow and clear and at about pH 6.9±0.2. Typically, this would mean diafiltering around 6-8 DVs.

The final dialyzed material undergoes 0.2 μm filtration and spray drying. The final step in the downstream process involves polishing wherein the final product is obtained by spray drying. However, prior to spray drying, the ultrafiltered retentate can be sterile filtered using a 0.2 μm MF filter. This needs to be done to reach our target specifications on the microload of our product.

Parameters are as provided in Table 9.

TABLE 9
0.2 um Tangential Flow Filtration parameters

| | |
|---|---|
| Membrane type | Polyether sulfone (PES) hollow |
| | fiber/spiral wound |
| Membrane size | 0.2 μm nominal |
| Temperature | 10° C. ± 5 |
| Final pH | 6.9 ± 0.2 |
| Transmembrane | Up to 2 bar (follow manufacturer |
| pressure (TMP) | recommendation) |
| Average flux (expected) | 10 ± 2 LMH |
| Color of final product | Clear yellow/gold |

The filtered material is then spray dried at the following conditions: Inlet temperature: 165° C., Outlet temperature: 65-67° C., and Air inlet: 3 bar.

The final powdered product shall meet the following specifications depending on the type of product, as summarized in Table 10.

TABLE 10
Specification of final product

Moisture content 5 to 10% w/w
Protein content >80% by bradford assay
Micro levels:
Standard Plate Count less than 5000 CFU/g
Yeast and Mold less than 10 CFU/g
Salmonella not detected in 25 g
E-coli not detected in 25 g
Heavy metals levels
Mercury <0.1 ppm
Arsenic <0.2 ppm
Lead <0.5 ppm
Cadmium <0.1 ppm

### 6.7. Example 7: Properties of an Illustrative Recovered Protein of Interest

In this example, properties of a recovered protein of interest were assayed. Here, the recovered protein of FIGS. 1A-1F in Example 1 was evaluated for functionality.

The recovered rOVA provided a foam capacity that was higher than a control OVA sample as shown in Table 11. The rOVA recovered provided increased foam capacity by: about 46.3% according to NES-016; about 57.1% accordingly to NES-019; and about 60.7% according to NES-021.

TABLE 11
Foaming and basic parameter evaluation

| | Foam | Foam | Conductivity | |
|---|---|---|---|---|
| Sample | Capacity | Stability | (mS/cm) | pH |
| Egg white | 350 | 73 | Not tested | 6.5 |
| powder (EWP) | | | | |
| 005 | 625 | 65 | 2.5 | 5.85 |
| 016 | 512.5 | 50 | 1.312 | 5.34 |
| 017 | 375 | 28.13 | 0.698 | 6.9 |
| 019 | 550 | 50 | 1.046 | 6.09 |
| 021 | 562.5 | 50 | 0.87 | 5.93 |

The recovered rOVA provided reduced hardness than a control OVA (EWP) sample as shown in Table 12. The rOVA recovered provided increased foam capacity by: about 59.7% according to NES-016; about 62.2% according to NES-017; about 28.3% according to NES-019; about 54.7% according to NES-021. NES-019 and NES-021 provided comparable springiness to the control OVA (EWP).

TABLE 12
Gelation evaluation

| | Hardness | Adhesiveness | Hardness | | Springiness | Guminess | Chewiness | Fracturability |
|---|---|---|---|---|---|---|---|---|
| Sample | 1 (g) | mJ | 2 g | Cohesiveness | mm | g | mJ | g |
| EWP | 758 | −3.6 | 708 | 50.8 | 0.82 | 38515 | 31465 | N/a |
| 005 | 449.2 | 0.82 | 404.87 | 0.58 | 4.6 | 258.93 | 11.77 | N/a |
| 016 | 305.29 | −13.29 | 261.23 | 44.95 | 0.65 | 13631 | 8911 | 240.12 |
| 017 | 286.81 | −20.57 | 212.92 | 22.75 | 0.47 | 6525 | 3066 | 282.55 |
| 019 | 542.93 | −1.93 | 471.04 | 47.91 | 0.84 | 25992 | 21926 | 542.93 |
| 021 | 343.23 | −3.35 | 268.49 | 42.07 | 0.83 | 14416 | 11968 | 343.23 |

Sensory evaluation of the recovered rOVA is as summarized in Table 13 below.

TABLE 13
Sensory evaluation

| | |
|---|---|
| 005 | Beige hue, opaque, some pores/air pockets, |
| | firm, compressed, did not stick to probe |
| 016 | moderate to strong beige color, not wet, |
| | very mild to mild firm, moderate to strong |
| | porous, mild to moderate smooth surface, |
| | mild crumbly, mild sulfur, mild brothy, |
| | mild umami |
| 017 | moderate to strong yellow, mild to moderate |
| | translucent, moderate to strong brittle, |
| | mild smooth surface, moderate to strong |
| | firm, moderate to strong crumbly, mild |
| | yeasty, mild to moderate brothy |
| 019 | moderate beige color, very mild pink hue, |
| | mild porous, moderate smooth surface, very |
| | mild crumbly, mild firm, mild to moderate |
| | cooked meat, mild mushroom, mild yeasty |
| 021 | moderate beige color, very mild pink hue, |
| | moderate porous, mild to moderate crumbly, |
| | mild smooth surface, moderate cooked |
| | mushroom, very mild yeasty |

### 6.8. Example 8: Properties of an Illustrative Baked Food Composition of the Recovered Protein of Interest

In this example, properties of a baked food composition of the recovered protein of interest were assayed. Here, the recovered protein of FIGS. 1A-1F in Example 1 and Example 6 was evaluated for physical measurements (Table 14), sensory appeal (Table 15), and volume (Table 16). The baked food composition is a pound cake.

Table 14 shows physical measurement of batter and cake. Regular pound cake containing the recovered rOVA provided comparable batter density (e.g., NES-007, NES-16, NES-19, NES-021), comparable pH of batter (e.g., NES-16, NES-19, NES-021), comparable moisture (e.g., NES-007, NES-16, NES-19, NES-021), comparable Aw (e.g., NES-007, NES-16, NES-17, NES-19, NES-021), comparable center height (e.g., NES-007, NES-16, NES-19, NES-021) or increased center height (e.g., NES-17).

TABLE 14
Physical measurement of batter and cake.

| | Batter | pH of | Moisture | | Center | |
|---|---|---|---|---|---|---|
| | density | Batter | % | Aw | height (mm) | Sensory |
| Control | 0.916 | 6.74 | 25.31 | 0.8833 | 68.85 ± 1.90 | Typical pound |
| | | | | | | cake. Moist |
| | | | | | | crumb with |
| | | | | | | vanilla flavoring |
| | | | | | | and smell. |
| NES-007 | 0.866 | | 25.45 | 0.852 | 66.21 | |
| NES-16 | 0.8321 | 6.75 | 25.25 | 0.872 | 67.07 ± 0.06 | Powder Texture |
| NES-17 | 0.779 | 7.23 | 23.36 | 0.818 | 74.80 | |
| NES-19 | 0.895 | 6.71 | 25.15 | 0.8390 | 68.80 ± 0.14 | N/A not |
| | | | | | | microclear |
| NES-21 | 0.907 | 6.56 | 25.54 | 0.8505 | 66.80 ± 0.70 | N/A not |
| | | | | | | microclear |

Gelation evaluation of the pound cake is summarized in Table 15. Regular pound cake containing the recovered rOVA provided comparable resilience, cohesiveness, and springiness (e.g., NES-16, NES-17, NES-19, NES-021); and improved chewiness (NES-7, about 32.1%; NES-16, about 12.7%; NES-19, about 16.5%; NES-021, 43.3%).

TABLE 15
TPA results of baked food composition

| | | | Cohe- | | |
|---|---|---|---|---|---|
| | Hardness | Resilience | siveness | Springiness | Chewiness |
| Control | 1134^{D} | 0.27^{A} | 0.67^{A} | 0.86^{A} | 65.3^{D} |
| NES-7 | 1597^{AB} | 0.28^{A} | 0.66^{AB} | 0.83^{A} | 86.3^{AB} |
| NES-16 | 1431^{BC} | 0.24^{B} | 0.61^{B} | 0.85^{A} | 73.6^{BCD} |
| NES-17 | 1319^{C} | 0.24^{AB} | 0.61^{BC} | 0.83^{A} | 66.5^{CD} |
| NES-19 | 1493^{BC} | 0.24^{B} | 0.60^{B} | 0.84^{A} | 76.1^{B} |
| NES-21 | 1732^{A} | 0.27^{AB} | 0.64^{BC} | 0.85^{A} | 93.6^{A} |

* Control: liquid whole egg. Typical hardness is around 1000 for control.

Table 16 shows volume of pound cake containing the recovered rOVA.

TABLE 16
Bake product volumn

| | | Specific | | Height |
|---|---|---|---|---|
| | Volume | Volume | Density | of Middle |
| | (mL) | (mL/g) | (kg/m2) | Slice (mm) |
| Control | 662.9 ± 8.0 | 2.244 ± 0.035 | 445.7 ± 6.9 | 70.8 ± 0.1 |
| NES-17 | 670.7 ± 8.8 | 2.257 ± 0.007 | 443.0 ± 1.5 | 75.3 ± 0.1 |
| NES-19 | 636.1 ± 1.68 | 2.102 ± 0.008 | 475.8 ± 1.8 | 69.4 ± 1.1 |
| NES-21 | 606.0 ± 10.7 | 2.017 ± 0.041 | 492.0 ± 9.89 | 68.3 ± 2.3 |

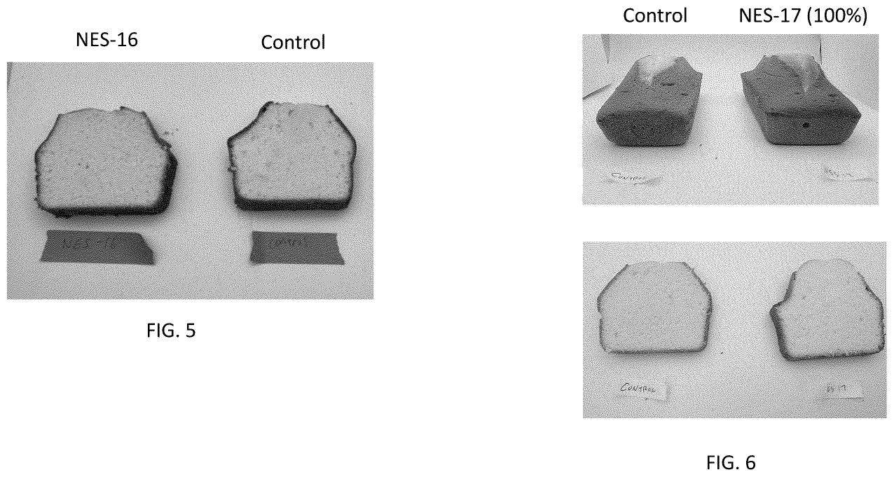

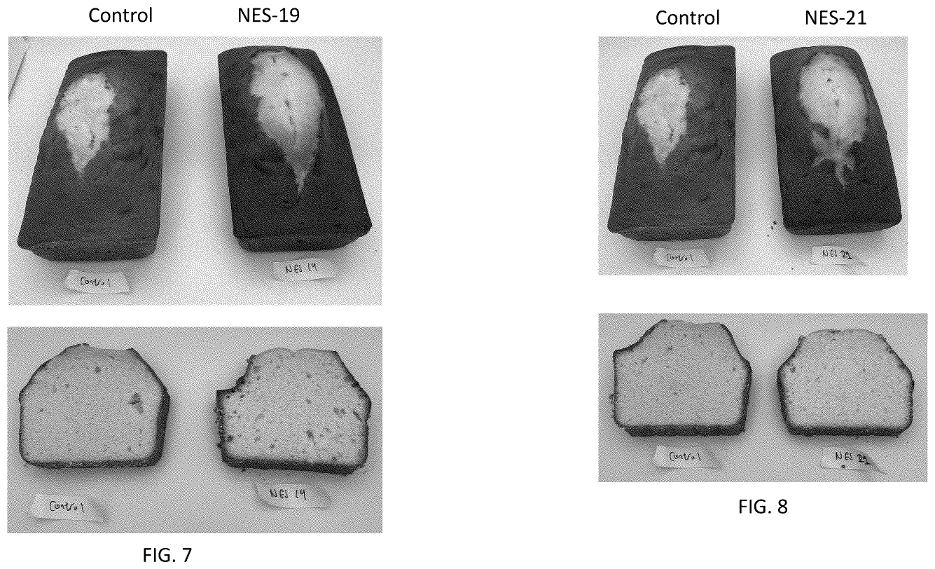

FIGS. 5-8 show photographs of pound cake containing the recovered rOVA according to NES-16, NES-17, NES-19, and NES-21, respectively.

### 6.9. Example 9: Properties of an Illustrative Non-Meat Food Composition of the Recovered Protein of Interest

In this example, properties of a non-meat food composition of the recovered protein of interest were assayed. Here, the recovered protein of FIGS. 1A-1F in Example 1 was evaluated for physical measurements (Table 17), and sensory appeal (Table 18). The non-meat food composition is burger.

Table 17 shows physical measurement of burger. Burger or patty containing the recovered rOVA provided comparable moisture (e.g., NES-007, NES-16, NES-17, NES-19, NES-021), and comparable Aw (e.g., NES-007, NES-16, NES-17, NES-19, NES-021). The raw patty was similar to control egg white protein, sticky, wet, held its shape, cohesive and not oily (e.g., NES-16, NES-17), or more wet and softer than EWP, and liquid spewed when pressed (e.g., NES-19, NES-021).

TABLE 17
Physical measurement of burger

| | Moisture | | | |
|---|---|---|---|---|
| Sample | % | Aw | Sensory | Raw patty handling |
| EWP | 49.23% | 0.9898 | Firm, chewy, no | Sticky, wet, holds its |
| | | | off-flavors | shape, cohesive, |
| | | | or scent | not oily |
| NES-007 | 47.28 | 0.9982 | Browning was darker | Softer and less smooth, |
| | | | and uneven, inside | cohesive, similar |
| | | | was lightened, and | stickiness, more oily |
| | | | observed cracks on | |
| | | | the surface. Did not | |
| | | | taste burgers | |
| NES-16 | 48.95% | 0.9957 | N/A | Similar to egg white |
| NES-17 | 47.24 | 0.9951 | N/A | Sticky, wet, holds its |
| | | | | shape, cohesive, |
| | | | | not oily |
| NES-19 | 49.26% | 0.9884 | N/A | More wet and softer than |
| | | | | EWP. Liquid spewed when |
| | | | | pressed. |
| DSP NES-21 | 49.26% | 0.9884 | N/A | More wet and softer than |
| | | | | EWP. Liquid spewed when |
| | | | | pressed. |

Table 18 shows TPA results on cooked burgers. Cooked burger or patty containing the recovered rOVA provided reduced hardness by: about 49.6% according to NES-007, about 6.2% according to NES-16, about 28.6% according to NES-016, about 14.5% according to NES-19, and about 21.3% according to NES-021.

TABLE 18
TPA results on cooked burgers

| | Hardness | Cohesiveness | Springiness | Chewiness |
|---|---|---|---|---|
| EWP | 6347.8 ± 355.71 | 0.374 ± 0.065 | 0.075 ± 0.019 | 172.726 ± 361.986 |
| *NES-007 | 3198.4 ± 668.45 | 0.19 ± 0.01 | 0.54 ± 0.07 | 33.45 ± 11.93 |
| NES-16 | 5948 ± 1095 | 0.238 ± 0.004 | 0.706 ± 0.106 | 103.9 ± 49.3 |
| NES-17 | 4534.112 ± 296.557 | 0.243 ± 0.016 | 0.007 ± 0.001 | 74.492 ± 17.459 |
| NES-19 | 5425.3 ± 439.7 | 0.283 ± 0.027 | 0.067 ± 0.001 | 103.696 ± 173.277 |
| NES-21 | 4992.3 ± 263.955 | 0.245 ± 0.016 | 0.065 ± 0.004 | 79.585 ± 113.455 |

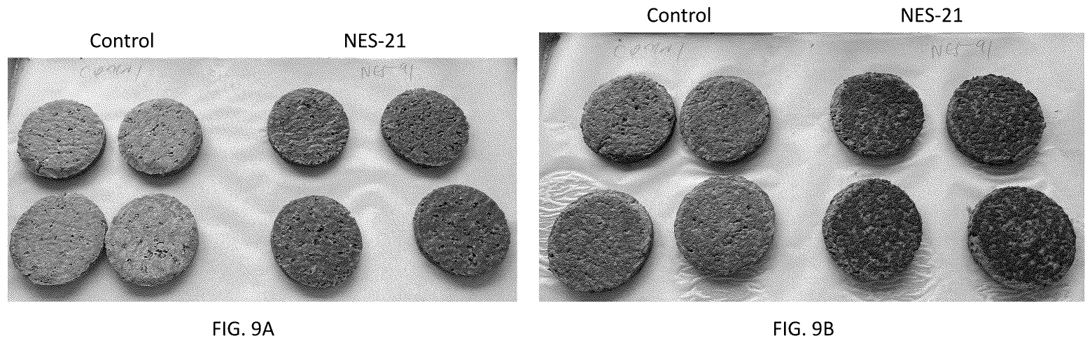

FIG. 9A shows a photograph of raw burger of control egg white protein and rOVA recovered according to NSE-021. FIG. 9B shows cooked burger of control egg white protein and rOVA recovered according to NSE-021. NES-021 burger had dark brown crust compared to control.

### 7. SEQUENCE LISTING

Illustrative OVA amino acid sequences contemplated herein are provided in the below Table 1 as SEQ ID NO: 1 to SEQ ID NO: 74.

TABLE 1
Sequence Listing

| SEQ | | |
|---|---|---|
| ID NO | Name | Sequence |
| 1 | Chicken | MRFPSIFTAVLFAASSALAAPVNTTTEDETAQIPAEAVIGYSDLEGDFDVAVLPFS |
| | Ovalbumin with | NSTNNGLLFINTTIASIAAKEEGVSLDKREAEAGSIGAASMEFCFDVFKELKVHHA |
| | bolded signal | NENIFYCPIAIMSALAMVYLGAKDSTRTQINKVVRFDKLPGFGDSIEAQCGTSVNV |
| | sequence | HSSLRDILNQITKPNDVYSFSLASRLYAEERYPILPEYLQCVKELYRGGLEPINFQT |
| | | AADQARELINSWVESQTNGIIRNVLQPSSVDSQTAMVLVNAIVFKGLWEKAFKDE |
| | | DTQAMPFRVTEQESKPVQMMYQIGLFRVASMASEKMKILELPFASGTMSMLVLL |
| | | PDEVSGLEQLESIINFEKLTEWTSSNVMEERKIKVYLPRMKMEEKYNLTSVLMAM |
| | | GITDVFSSSANLSGISSAESLKISQAVHAAHAEINEAGREVVGSAEAGVDAASVSEE |
| | | FRADHPFLFCIKHIATNAVLFFGRCVSP |
| 2 | Chicken OVA | EAEAGSIGAASMEFCFDVFKELKVHHANENIFYCPIAIMSALAMVYLGAKDSTRT |
| | sequence as | QINKVVRFDKLPGFGDSIEAQCGTSVNVHSSLRDILNQITKPNDVYSFSLASRLYA |
| | secreted from | EERYPILPEYLQCVKELYRGGLEPINFQTAADQARELINSWVESQINGIIRNVLQPS |
| | pichia | SVDSQTAMVLVNAIVFKGLWEKAFKDEDTQAMPFRVTEQESKPVQMMYQIGLF |
| | | RVASMASEKMKILELPFASGTMSMLVLLPDEVSGLEQLESIINFEKLTEWTSSNV |
| | | MEERKIKVYLPRMKMEEKYNLTSVLMAMGITDVFSSSANLSGISSAESLKISQAV |
| | | HAAHAEINEAGREVVGSAEAGVDAASVSEEFRADHPFLFCIKHIATNAVLFFGRC |
| | | VSP |
| 3 | Predicted | MRVPAQLLGLLLLWLPGARCGSIGAASMEFCFDVFKELKVHHANENIFYCPIAIM |
| | Ovalbumin | SALAMVYLGAKDSTRTQINKVVRFDKLPGFGDSIEAQCGTSVNVHSSLRDILNQIT |
| | [Achromobacter | KPNDVYSFSLASRLYAEERYPILPEYLQCVKELYRGGLEPINFQTAADQARELINS |
| | denitrificans] | WVESQINGIIRNVLQPSSVDSQTAMVLVNAIVFKGLWEKAFKDEDTQAMPFRVT |
| | | EQESKPVQMMYQIGLFRVASMASEKMKILELPFASGTMSMLVLLPDEVSGLEQL |
| | | ESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLF |
| | | CIKHIATNAVLFFGRCVSPLEIKRAAAHHHHHH |
| 4 | OLLAS epitope- | MTSGFANELGPRLMGKLTMGSIGAASMEFCFDVFKELKVHHANENIFYCPIAIMS |
| | tagged ovalbumin | ALAMVYLGAKDSTRTQINKVVRFDKLPGFGDSIEAQCGTSVNVHSSLRDILNQIT |
| | | KPNDVYSFSLASRLYAEERYPILPEYLQCVKELYRGGLEPINFQTAADQARELINS |
| | | WVESQTNGIIRNVLQPSSVDSQTAMVLVNAIVFKGLWEKTFKDEDTQAMPFRVT |
| | | EQESKPVQMMYQIGLFRVASMASEKMKILELPFASGTMSMLVLLPDEVSGLEQL |
| | | ESIINFEKLTEWTSSNVMEERKIKVYLPRMKMEEKYNLTSVLMAMGITDVFSSSA |
| | | NLSGISSAESLKISQAVHAAHAEINEAGREVVGSAEAGVDAASVSEEFRADHPFLF |
| | | CIKHIATNAVLFFGRCVSPSR |
| 5 | Serpin family | MGGRRVRWEVYISRAGYVNRQIAWRRHHRSLTMRVPAQLLGLLLLWLPGARCG |
| | protein | SIGAASMEFCFDVFKELKVHHANENIFYCPIAIMSALAMVYLGAKDSTRTQINKV |
| | [Achromobacter | VRFDKLPGFGDSIEAQCGTSVNVHSSLRDILNQITKPNDVYSFSLASRLYAEERYPI |
| | denitrificans] | LPEYLQCVKELYRGGLEPINFQTAADQARELINSWVESQINGIIRNVLQPSSVDSQ |
| | | TAMVLVNAIVFKGLWEKAFKDEDTQAMPFRVTEQESKPVQMMYQIGLFRVASM |
| | | ASEKMKILELPFASGTMSMLVLLPDEVSGLEQLESIINFEKLTEWTSSNVMEERKI |
| | | KVYLPRMKMEEKYNLTSVLMAMGITDVFSSSANLSGISSAESLKISQAVHAAHAE |
| | | INEAGREVVGSAEAGVDAASVSEEFRADHPFLFCIKHIATNAVLFFGRCVSPLEIKR |
| | | AAAHHHHHH |
| 6 | PREDICTED: | MGSIGAVSMEFCFDVFKELKVHHANENIFYSPFTIISALAMVYLGAKDSTRTQINK |
| | ovalbumin | VVRFDKLPGFGDSVEAQCGTSVNVHSSLRDILNQITKPNDVYSFSLASRLYAEETY |
| | isoform X1 | PILPEYLQCVKELYRGGLESINFQTAADQARGLINSWVESQTNGMIKNVLQPSSV |
| | [Meleagris | DSQTAMVLVNAIVFKGLWEKAFKDEDTQAIPFRVTEQESKPVQMMYQIGLFKVA |
| | gallopavo] | SMASEKMKILELPFASGTMSMWVLLPDEVSGLEQLETTISFEKMTEWISSNIMEER |
| | | RIKVYLPRMKMEEKYNLTSVLMAMGITDLFSSSANLSGISSAGSLKISQAVHAAY |
| | | AEIYEAGREVIGSAEAGADATSVSEEFRVDHPFLYCIKHNLTNSILFFGRCISP |
| 7 | Ovalbumin | MGSIGAVSMEFCFDVFKELKVHHANENIFYSPFTIISALAMVYLGAKDSTRTQINK |
| | precursor | VVRFDKLPGFGDSVEAQCGTSVNVHSSLRDILNQITKPNDVYSFSLASRLYAEETY |
| | [Meleagris | PILPEYLQCVKELYRGGLESINFQTAADQARGLINSWVESQTNGMIKNVLQPSSV |
| | gallopavo] | DSQTAMVLVNAIVFKGLWEKAFKDEDTQAIPFRVTEQESKPVQMMYQIGLFKVA |
| | | SMASEKMKILELPFASGTMSMWVLLPDEVSGLEQLETTISFEKMTEWISSNIMEER |
| | | RIKVYLPRMKMEEKYNLTSVLMAMGITDLFSSSANLSGISSAGSLKISQAAHAAY |
| | | AEIYEAGREVIGSAEAGADATSVSEEFRVDHPFLYCIKHNLTNSILFFGRCISP |
| 8 | Hypothetical | YYRVPCMVLCTAFHPYIFIVLLFALDNSEFTMGSIGAVSMEFCFDVFKELRVHHPN |
| | protein | ENIFFCPFAIMSAMAMVYLGAKDSTRTQINKVIRFDKLPGFGDSTEAQCGKSANV |
| | [Bambusicola | HSSLKDILNQITKPNDVYSFSLASRLYADETYSIQSEYLQCVNELYRGGLESINFQT |
| | thoracicus] | AADQARELINSWVESQINGIIRNVLQPSSVDSQTAMVLVNAIVFRGLWEKAFKDE |
| | | DTQTMPFRVTEQESKPVQMMYQIGSFKVASMASEKMKILELPLASGTMSMLVLL |
| | | PDEVSGLEQLETTISFEKLTEWTSSNVMEERKIKVYLPRMKMEEKYNLTSVLMA |
| | | MGITDLFRSSANLSGISLAGNLKISQAVHAAHAEINEAGRKAVSSAEAGVDATSVS |
| | | EEFRADRPFLFCIKHIATKVVFFFGRYTSP |
| 9 | Egg albumin | MGSIGAASMEFCFDVFKELKVHHANDNMLYSPFAILSTLAMVFLGAKDSTRTQIN |
| | | KVVHFDKLPGFGDSIEAQCGTSVNVHSSLRDILNQITKQNDAYSFSLASRLYAQET |
| | | YTVVPEYLQCVKELYRGGLESVNFQTAADQARGLINAWVESQINGIIRNILQPSS |
| | | VDSQTAMVLVNAIAFKGLWEKAFKAEDTQTIPFRVTEQESKPVQMMYQIGSFKV |
| | | ASMASEKMKILELPFASGTMSMLVLLPDDVSGLEQLESIISFEKLTEWTSSSIMEER |
| | | KVKVYLPRMKMEEKYNLTSLLMAMGITDLFSSSANLSGISSVGSLKISQAVHAAH |
| | | AEINEAGRDVVGSAEAGVDATEEFRADHPFLFCVKHIETNAILLFGRCVSP |
| 10 | Ovalbumin | MASIGAVSTEFCVDVYKELRVHHANENIFYSPFTIISTLAMVYLGAKDSTRTQINK |
| | isoform X2 | VVRFDKLPGFGDSIEAQCGTSVNVHSSLRDILNQITKPNDVYSFSLASRLYAEETY |
| | [Numida | PILPEYLQCVKELYRGGLESINFQTAADQARELINSWVESQTSGIIKNVLQPSSVNS |
| | meleagris] | QTAMVLVNAIYFKGLWERAFKDEDTQAIPFRVTEQESKPVQMMSQIGSFKVASV |
| | | ASEKVKILELPFVSGTMSMLVLLPDEVSGLEQLESTISTEKLTEWTSSSIMEERKIK |
| | | VFLPRMRMEEKYNLTSVLMAMGMTDLFSSSANLSGISSAESLKISQAVHAAYAEI |
| | | YEAGREVVSSAEAGVDATSVSEEFRVDHPFLLCIKHNPTNSILFFGRCISP |
| 11 | Ovalbumin | MALCKAFHPYIFIVLLFDVDNSAFTMASIGAVSTEFCVDVYKELRVHHANENIFYS |
| | isoform X1 | PFTIISTLAMVYLGAKDSTRTQINKVVRFDKLPGFGDSIEAQCGTSVNVHSSLRDIL |
| | [Numida | NQITKPNDVYSFSLASRLYAEETYPILPEYLQCVKELYRGGLESINFQTAADQARE |
| | meleagris] | LINSWVESQTSGIIKNVLQPSSVNSQTAMVLVNAIYFKGLWERAFKDEDTQAIPFR |
| | | VTEQESKPVQMMSQIGSFKVASVASEKVKILELPFVSGTMSMLVLLPDEVSGLEQ |
| | | LESTISTEKLTEWTSSSIMEERKIKVFLPRMRMEEKYNLTSVLMAMGMTDLFSSSA |
| | | NLSGISSAESLKISQAVHAAYAEIYEAGREVVSSAEAGVDATSVSEEFRVDHPFLL |
| | | CIKHNPTNSILFFGRCISP |
| 12 | PREDICTED: | MGSIGAASMEFCFDVFKELKVHHANDNMLYSPFAILSTLAMVFLGAKDSTRTQIN |
| | Ovalbumin | KVVHFDKLPGFGDSIEAQCGTSANVHSSLRDILNQITKQNDAYSFSLASRLYAQET |
| | isoform X2 | YTVVPEYLQCVKELYRGGLESVNFQTAADQARGLINAWVESQTNGIIRNILQPSS |
| | [Coturnix | VDSQTAMVLVNAIAFKGLWEKAFKAEDTQTIPFRVTEQESKPVQMMHQIGSFKV |
| | japonica] | ASMASEKMKILELPFASGTMSMLVLLPDDVSGLEQLESTISFEKLTEWTSSSIMEE |
| | | RKVKVYLPRMKMEEKYNLTSLLMAMGITDLFSSSANLSGISSVGSLKISQAVHAA |
| | | YAEINEAGRDVVGSAEAGVDATEEFRADHPFLFCVKHIETNAILLFGRCVSP |
| 13 | PREDICTED: | MGLCTAFHPYIFIVLLFALDNSEFTMGSIGAASMEFCFDVFKELKVHHANDNMLY |
| | ovalbumin | SPFAILSTLAMVFLGAKDSTRTQINKVVHFDKLPGFGDSIEAQCGTSANVHSSLRD |
| | isoform X1 | ILNQITKQNDAYSFSLASRLYAQETYTVVPEYLQCVKELYRGGLESVNFQTAADQ |
| | [Coturnix | ARGLINAWVESQTNGIIRNILQPSSVDSQTAMVLVNAIAFKGLWEKAFKAEDTQTI |
| | japonica] | PFRVTEQESKPVQMMHQIGSFKVASMASEKMKILELPFASGTMSMLVLLPDDVS |
| | | GLEQLESTISFEKLTEWTSSSIMEERKVKVYLPRMKMEEKYNLTSLLMAMGITDL |
| | | FSSSANLSGISSVGSLKISQAVHAAYAEINEAGRDVVGSAEAGVDATEEFRADHPF |
| | | LFCVKHIETNAILLFGRCVSP |
| 14 | Egg albumin | MGSIGAASMEFCFDVFKELKVHHANDNMLYSPFAILSTLAMVFLGAKDSTRTQIN |
| | | KVVHFDKLPGFGDSIEAQCGTSANVHSSLRDILNQITKQNDAYSFSLASRLYAQET |
| | | YTVVPEYLQCVKELYRGGLESVNFQTAADQARGLINAWVESQINGIIRNILQPSS |
| | | VDSQTAMVLVNAIAFKGLWEKAFKAEDTQTIPFRVTEQESKPVQMMHQIGSFKV |
| | | ASMASEKMKILELPFASGTMSMLVLLPDDVSGLEQLESTISFEKLTEWTSSSIMEE |
| | | RKVKVYLPRMKMEEKYNLTSLLMAMGITDLFSSSANLSGISSVGSLKIPQAVHAA |
| | | YAEINEAGRDVVGSAEAGVDATEEFRADHPFLFCVKHIETNAILLFGRCVSP |
| 15 | ovalbumin [Anas | MGSIGAASTEFCFDVFRELRVQHVNENIFYSPFSIISALAMVYLGARDNTRTQIDK |
| | platyrhynchos] | VVHFDKLPGFGESMEAQCGTSVSVHSSLRDILTQITKPSDNFSLSFASRLYAEETY |
| | | AILPEYLQCVKELYKGGLESISFQTAADQARELINSWVESQINGIIKNILQPSSVDS |
| | | QTTMVLVNAIYFKGMWEKAFKDEDTQAMPFRMTEQESKPVQMMYQVGSFKVA |
| | | MVTSEKMKILELPFASGMMSMFVLLPDEVSGLEQLESTISFEKLTEWTSSTMMEE |
| | | RRMKVYLPRMKMEEKYNLTSVFMALGMTDLFSSSANMSGISSTVSLKMSEAVH |
| | | AACVEIFEAGRDVVGSAEAGMDVTSVSEEFRADHPFLFFIKHNPTNSILFFGRWMS |
| | | P |
| 16 | PREDICTED: | MGSIGAASTEFCFDVFRELKVQHVNENIFYSPLSIISALAMVYLGARDNTRTQIDQ |
| | ovalbumin-like | VVHFDKIPGFGESMEAQCGTSVSVHSSLRDILTEITKPSDNFSLSFASRLYAEETYT |
| | [Anser cygnoides | ILPEYLQCVKELYKGGLESISFQTAADQARELINSWVESQINGIIKNILQPSSVDSQ |
| | domesticus] | TTMVLVNAIYFKGMWEKAFKDEDTQTMPFRMTEQESKPVQMMYQVGSFKLAT |
| | | VTSEKVKILELPFASGMMSMCVLLPDEVSGLEQLETTISFEKLTEWTSSTMMEER |
| | | RMKVYLPRMKMEEKYNLTSVFMALGMTDLFSSSANMSGISSTVSLKMSEAVHA |
| | | ACVEIFEAGRDVVGSAEAGMDVTSVSEEFRADHPFLFFIKHNPSNSILFFGRWISP |
| 17 | PREDICTED: | MGSIGAASTEFCFDVFKELKVQHVNENIFYSPLTIISALSMVYLGARENTRAQIDK |
| | Ovalbumin-like | VLHFDKMPGFGDTIESQCGTSVSIHTSLKDMFTQITKPSDNYSLSFASRLYAEETY |
| | [Aquila | PILPEYLQCVKELYKGGLETISFQTAAEQARELINSWVESQTNGMIKNILQPSSVDP |
| | chrysaetos | QTKMVLVNAIYFKGVWEKAFKDEDTQEVPFRVTEQESKPVQMMYQIGSFKVAV |
| | canadensis] | MASEKMKILELPYASGQLSMLVLLPDDVSGLEQLESAITFEKLMAWTSSTTMEER |
| | | KMKVYLPRMKIEEKYNLTSVLMALGVTDLFSSSANLSGISSAESLKISKAVHEAFV |
| | | EIYEAGSEVVGSTEAGMEVTSVSEEFRADHPFLFLIKHNPTNSILFFGRCFSP |
| 18 | PREDICTED: | MGSIGAASTEFCFDVFKELKVQHVNENIFYSPLTIISALSMVYLGARENTRTQIDK |
| | Ovalbumin-like | VLHFDKMTGFGDTVESQCGTSVSIHTSLKDIFTQITKPSDNYSLSLASRLYAEETYP |
| | [Haliaeetus | ILPEYLQCVKELYKGGLETVSFQTAAEQARELINSWVESQTNGMIKNILQPSSVDP |
| | albicilla] | QTKMVLVNAIYFKGVWEKAFKDEDTQEVPFRVTEQESKPVQMMYQIGSFKVAV |
| | | MASEKMKILELPYASGQLSMLVLLPDDVSGLEQLESAITSEKLMEWTSSTTMEER |
| | | KMKVYLPRMKIEEKYNLTSVLMALGVTDLFSSSADLSGISSAESLKISKAVHEAFV |
| | | EIYEAGSEVVGSTEGGMEVTSVSEEFRADHPFLFLIKHKPTNSILFFGRCFSP |
| 19 | PREDICTED: | MGSIGAASTEFCFDVFKELKVQHVNENIFYSPLTIISALSMVYLGARENTRTQIDK |
| | Ovalbumin-like | VLHFDKMTGFGDTVESQCGTSVSIHTSLKDIFTQITKPSDNYSLSLASRLYAEETYP |
| | [Haliaeetus | ILPEYLQCVKELYKGGLETVSFQTAAEQARELINSWVESQTNGMIKNILQPSSVDP |
| | leucocephalus] | QTKMVLVNAIYFKGVWEKAFKDEDTQEVPFRVTEQESKPVQMMYQIGSFKVAV |
| | | MASEKMKILELPYASGQLSMLVLLPDDVSGLEQLESAITSEKLMEWTSSTTMEER |
| | | KMKVYLPRMKIEEKYNLTSVLMALGVTDLFSSSADLSGISSAESLKISKAVHEAFV |
| | | EIYEAGSEVVGSTEGGMEVTSFSEEFRADHPFLFLIKHKPTNSILFFGRCFSP |
| 20 | PREDICTED: | MGSIGAASTEFCFDVFKELKVQHVNENIFYSPLSIISALSMVYLGARENTRAQIDK |
| | Ovalbumin | VVHFDKITGFGETIESQCGTSVSVHTSLKDMFTQITKPSDNYSLSFASRLYAEETYP |
| | [Fulmarus | ILPEYLQCVKELYKGGLETTSFQTAADQARELINSWVESQTNGMIKNILQPGSVDP |
| | glacialis] | QTEMVLVNAIYFKGMWEKAFKDEDTQAVPFRMTEQESKTVQMMYQIGSFKVAV |
| | | MASEKMKILELPYASGELSMLVMLPDDVSGLEQLETAITFEKLMEWTSSNMMEE |
| | | RKMKVYLPRMKMEEKYNLTSVLMALGVTDLFSSSANLSGISSAESLKMSEAVHE |
| | | AFVEIYEAGSEVVGSTGAGMEVTSVSEEFRADHPFLFLIKHNPTNSILFFGRCFSP |
| 21 | PREDICTED: | MGSIGAASTEFCFDVFKELRVQHVNENVCYSPLIIISALSLVYLGARENTRAQIDK |
| | Ovalbumin-like | VVHFDKITGFGESIESQCGTSVSVHTSLKDMFNQITKPSDNYSLSVASRLYAEERY |
| | [Chlamydotis | PILPEYLQCVKELYKGGLESISFQTAADQAREAINSWVESQTNGMIKNILQPSSVD |
| | macqueenii] | PQTEMVLVNAIYFKGMWQKAFKDEDTQAVPFRISEQESKPVQMMYQIGSFKVAV |
| | | MAAEKMKILELPYASGELSMLVLLPDEVSGLEQLENAITVEKLMEWTSSSPMEER |
| | | IMKVYLPRMKIEEKYNLTSVLMALGITDLFSSSANLSGISAEESLKMSEAVHQAFA |
| | | EISEAGSEVVGSSEAGIDATSVSEEFRADHPFLFLIKHNATNSILFFGRCFSP |
| 22 | PREDICTED: | MGSISAASTEFCFDVFKELKVQHVNENIFYSPLSIISALSMVYLGARENTRAQIEKV |
| | Ovalbumin like | VHFDKITGFGESIESQCSTSVSVHTSLKDMFTQITKPSDNYSLSFASRFYAEETYPIL |
| | [Nipponia | PEYLQCVKELYKGGLETINFRTAADQARELINSWVESQTNGMIKNILQPGSVDPQ |
| | nippon] | TDMVLVNAIYFKGMWEKAFKDEDTQALPFRVTEQESKPVQMMYQIGSFKVAVL |
| | | ASEKVKILELPYASGQLSMLVLLPDDVSGLEQLETAITVEKLMEWTSSNNMEERK |
| | | IKVYLPRIKIEEKYNLTSVLMALGITDLFSSSANLSGISSAESLKVSEAIHEAFVEIYE |
| | | AGSEVAGSTEAGIEVTSVSEEFRADHPFLFLIKHNATNSILFFGRCFSP |
| 23 | PREDICTED: | MVSIGAASTEFCFDVFKELKVQHVNENIFYSPLSIISALSMVYLGARENTRAQIDK |
| | Ovalbumin-like | VVHFDKITGFEETIESQCSTSVSVHTSLKDMFTQITKPSDNYSLSFASRLYAEETYPI |
| | isoform X2 | LPEYLQCVKELYKGGLETISFQTAADQARELINSWVESQTDGMIKNILQPGSVDP |
| | [Gavia stellata] | QTEMVLVNAIYFKGMWEKAFKDEDTQAVPFRMTEQESKPVQMMYQIGSFKVAV |
| | | MASEKMKILELPYASGGMSMLVMLPDDVSGLEQLETAITFEKLMEWTSSNMME |
| | | ERKMKVYLPRMKMEEKYNLTSVLMALGMTDLFSSSANLSGISSAESLKMSEAVH |
| | | EAFVEIYEAGSEAVGSTGAGMEVTSVSEEFRADHPFLFLIKHNPTNSILFFGRCFSP |
| 24 | PREDICTED: | MGSIGAASTEFCFDVFKELKVQHVNENIFYSPLSIISALSMVYLGARENTRAQIDK |
| | Ovalbumin | VVHFDKITGFGEPIESQCGISVSVHTSLKDMITQITKPSDNYSLSFASRLYAEETYPI |
| | [Pelecanus | LPEYLQCVKELYKGGLETISFQTAADQARELINSWVENQTNGMIKNILQPGSVDP |
| | crispus] | QTEMVLVNAVYFKGMWEKAFKDEDTQAVPFRMTEQESKPVQMMYQIGSFKVA |
| | | VMASEKIKILELPYASGELSMLVLLPDDVSGLEQLETAITLDKLTEWTSSNAMEER |
| | | KMKVYLPRMKIEKKYNLTSVLIALGMTDLFSSSANLSGISSAESLKMSEAIHEAFL |
| | | EIYEAGSEVVGSTEAGMEVTSVSEEFRADHPFLFLIKHNPTNSILFFGRCLSP |
| 25 | PREDICTED: | MGSIGAASTEFCFDVFKELKVQHVNENIFYSPLTIISALSMVYLGARENTRAQIDK |
| | Ovalbumin-like | VVHFDKIPGFGDTTESQCGTSVSVHTSLKDMFTQITKPSDNYSVSFASRLYAEETY |
| | [Charadrius | PILPEFLECVKELYKGGLESISFQTAADQARELINSWVESQTNGMIKNILQPGSVDS |
| | vociferus] | QTEMVLVNAIYFKGMWEKAFKDEDTQTVPFRMTEQETKPVQMMYQIGTFKVAV |
| | | MPSEKMKILELPYASGELCMLVMLPDDVSGLEELESSITVEKLMEWTSSNMMEE |
| | | RKMKVFLPRMKIEEKYNLTSVLMALGMTDLFSSSANLSGISSAEPLKMSEAVHEA |
| | | FIEIYEAGSEVVGSTGAGMEITSVSEEFRADHPFLFLIKHNPTNSILFFGRCVSP |
| 26 | PREDICTED: | MGSIGAVSTEFCFDVFKELKVQHVNENIFYSPLSIISALSMVYLGARENTRAQIDK |
| | Ovalbumin-like | VVHFDKITGSGETIEAQCGTSVSVHTSLKDMFTQITKPSENYSVGFASRLYADETY |
| | [Eurypyga helias] | PIIPEYLQCVKELYKGGLEMISFQTAADQARELINSWVESQTNGMIKNILQPGSVD |
| | | PQTEMILVNAIYFKGVWEKAFKDEDTQAVPFRMTEQESKPVQMMYQFGSFKVA |
| | | AMAAEKMKILELPYASGALSMLVLLPDDVSGLEQLESAITFEKLMEWTSSNMME |
| | | EKKIKVYLPRMKMEEKYNFTSVLMALGMTDLFSSSANLSGISSADSLKMSEVVHE |
| | | AFVEIYEAGSEVVGSTGSGMEAASVSEEFRADHPFLFLIKHNPTNSILFFGRCFSP |
| 27 | PREDICTED: | MVSIGAASTEFCFDVFKELKVQHVNENIFYSPLSIISALSMVYLGARENTRAQIDK |
| | Ovalbumin-like | VVHFDKITGFEETIESQVQKKQCSTSVSVHTSLKDMFTQITKPSDNYSLSFASRLY |
| | isoform X1 | AEETYPILPEYLQCVKELYKGGLETISFQTAADQARELINSWVESQTDGMIKNILQ |
| | [Gavia stellata] | PGSVDPQTEMVLVNAIYFKGMWEKAFKDEDTQAVPFRMTEQESKPVQMMYQIG |
| | | SFKVAVMASEKMKILELPYASGGMSMLVMLPDDVSGLEQLETAITFEKLMEWTS |
| | | SNMMEERKMKVYLPRMKMEEKYNLTSVLMALGMTDLFSSSANLSGISSAESLK |
| | | MSEAVHEAFVEIYEAGSEAVGSTGAGMEVTSVSEEFRADHPFLFLIKHNPTNSILFF |
| | | GRCFSP |
| 28 | PREDICTED: | MGSIGAASGEFCFDVFKELKVQHVNENIFYSPLSIISALSMVYLGARENTRAQIDK |
| | Ovalbumin-like | VVHFDKIIGFGESIESQCGTSVSVHTSLKDMFAQITKPSDNYSLSFASRLYAEETFPI |
| | [Egretta garzetta] | LPEYLQCVKELYKGGLETLSFQTAADQARELINSWVESQTNGMIKDILQPGSVDP |
| | | QTEMVLVNAIYFKGVWEKAFKDEDTQTVPFRMTEQESKPVQMMYQIGSFKVAV |
| | | VAAEKIKILELPYASGALSMLVLLPDDVSSLEQLETAITFEKLTEWTSSNIMEERKI |
| | | KVYLPRMKIEEKYNLTSVLMDLGITDLFSSSANLSGISSAESLKVSEAIHEAIVDIYE |
| | | AGSEVVGSSGAGLEGTSVSEEFRADHPFLFLIKHNPTSSILFFGRCFSP |
| 29 | PREDICTED: | MGSIGAASTEFCFDVFKELKVQHVNENIFYSPLSIISALSMVYLGARENTRAQIDK |
| | Ovalbumin-like | VVHFDKITGSGEAIESQCGTSVSVHISLKDMFTQITKPSDNYSLSFASRLYAEETYP |
| | [Balearica | ILPEYLQCVKELYKEGLATISFQTAADQAREFINSWVESQTNGMIKNILQPGSVDP |
| | regulorum | QTQMVLVNAIYFKGVWEKAFKDEDTQAVPFRMTKQESKPVQMMYQIGSFKVAV |
| | gibbericeps] | MASEKMKILELPYASGQLSMLVMLPDDVSGLEQIENAITFEKLMEWTNPNMMEE |
| | | RKMKVYLPRMKMEEKYNLTSVLMALGMTDLFSSSANLSGISSAESLKMSEAVHE |
| | | AFVEIYEAGSEVVGSTGAGIEVTSVSEEFRADHPFLFLIKHNPTNSILFFGRCFSP |
| 30 | PREDICTED: | MGSIGEASTEFCIDVFRELKVQHVNENIFYSPLSIISALSMVYLGARENTRAQIDQV |
| | Ovalbumin-like | VHFDKITGFGDTVESQCGSSLSVHSSLKDIFAQITQPKDNYSLNFASRLYAEETYPI |
| | [Nestor notabilis] | LPEYLQCVKELYKGGLETISFQTAADQARELINSWVESQTNGMIKNILQPSSVDPQ |
| | | TEMVLVNAIYFKGVWEKAFKDEETQAVPFRITEQENRPVQIMYQFGSFKVAVVA |
| | | SEKIKILELPYASGQLSMLVLLPDEVSGLEQLENAITFEKLTEWTSSDIMEEKKIKV |
| | | FLPRMKIEEKYNLTSVLVALGIADLFSSSANLSGISSAESLKMSEAVHEAFVEIYEA |
| | | GSEVVGSSGAGIEAASDSEEFRADHPFLFLIKHKPTNSILFFGRCFSP |
| 31 | PREDICTED: | MGSIGAASTEFCFDIFNELKVQHVNENIFYSPLSIISALSMVYLGARENTKAQIDKV |
| | Ovalbumin-like | VHFDKITGFGESIESQCSTSASVHTSFKDMFTQITKPSDNYSLSFASRLYAEETYPIL |
| | [Pygoscelis | PEYSQCVKELYKGGLESISFQTAADQARELINSWVESQTNGMIKNILQPGSVDPQT |
| | adeliae] | ELVLVNAIYFKGTWEKAFKDKDTQAVPFRVTEQESKPVQMMYQIGSYKVAVIAS |
| | | EKMKILELPYASGELSMLVLLPDDVSGLEQLETAITFEKLMEWTSSNMMEERKV |
| | | KVYLPRMKIEEKYNLTSVLMALGMTDLFSPSANLSGISSAESLKMSEAIHEAFVEI |
| | | YEAGSEVVGSTEAGMEVTSVSEEFRADHPFLFLIKCNLTNSILFFGRCFSP |
| 32 | Ovalbumin-like | MGSISTASTEFCFDVFKELKVQHVNENIFYSPLSIISALSMVYLGARENTRAQIEKV |
| | [Athene | VHFDKITGFGESIESQCGTSVSVHTSLKDMLIQISKPSDNYSLSFASKLYAEETYPIL |
| | cunicularia] | PEYLQCVKELYKGGLESINFQTAADQARQLINSWVESQTNGMIKDILQPSSVDPQ |
| | | TEMVLVNAIYFKGIWEKAFKDEDTQEVPFRITEQESKPVQMMYQIGSFKVAVIAS |
| | | EKIKILELPYASGELSMLIVLPDDVSGLEQLETAITFEKLIEWTSPSIMEERKTKVYL |
| | | PRMKIEEKYNLTSVLMALGMTDLFSPSANLSGISSAESLKMSEAIHEAFVEIYEAGS |
| | | EVVGSAEAGMEATSVSEFRVDHPFLFLIKHNPANIILFFGRCVSP |
| 33 | REDICTED: | MGSIGAASTEFCFDVFKELKVQHVNENIFYSPLTIISALSLVYLGARENTRAQIDKV |
| | Ovalbumin-like | FHFDKISGFGETTESQCGTSVSVHTSLKEMFTQITKPSDNYSVSFASRLYAEDTYPI |
| | [Calidris pugnax] | LPEYLQCVKELYKGGLETISFQTAADQAREVINSWVESQTNGMIKNILQPGSVDS |
| | | QTEMVLVNAIYFKGMWEKAFKDEDTQTMPFRITEQERKPVQMMYQAGSFKVAV |
| | | MASEKMKILELPYASGEFCMLIMLPDDVSGLEQLENSFSFEKLMEWTTSNMMEE |
| | | RKMKVYIPRMKMEEKYNLTSVLMALGMTDLFSSSANLSGISSAETLKMSEAVHE |
| | | AFMEIYEAGSEVVGSTGSGAEVTGVYEEFRADHPFLFLVKHKPTNSILFFGRCVSP |
| 34 | PREDICTED: | MGSIGAASTEFCFDIFNELKVQHVNENIFYSPLSIISALSMVYLGARENTKAQIDKV |
| | Ovalbumin | VHFDKITGFGETIESQCSTSVSVHTSLKDTFTQITKPSDNYSLSFASRLYAEETYPIL |
| | [Aptenodytes | PEYSQCVKELYKGGLETISFQTAADQARELINSWVESQTNGMIKNILQPGSVDPQT |
| | forsteri] | ELVLVNAIYFKGTWEKAFKDKDTQAVPFRVTEQESKPVQMMYQIGSYKVAVIAS |
| | | EKMKILELPYASRELSMLVLLPDDVSGLEQLETAITFEKLMEWTSSNMMEERKVK |
| | | VYLPRMKIEEKYNLTSVLMALGMTDLFSPSANLSGISSAESLKMSEAVHEAFVEIY |
| | | EAGSEVVGSTGAGMEVTSVSEEFRADHPFLFLIKCNPTNSILFFGRCFSP |
| 35 | PREDICTED: | MGSISAASAEFCLDVFKELKVQHVNENIFYSPLSIISALSMVYLGARENTRAQIDK |
| | Ovalbumin-like | VVHFDKITGSGETIEFQCGTSANIHPSLKDMFTQITRLSDNYSLSFASRLYAEERYP |
| | [Pterocles | ILPEYLQCVKELYKGGLETISFQTAADQARELINSWVESQTNGMIKNILQPGSVNP |
| | gutturalis] | QTEMVLVNAIYFKGLWEKAFKDEDTQTVPFRMTEQESKPVQMMYQVGSFKVAV |
| | | MASDKIKILELPYASGELSMLVLLPDDVTGLEQLETSITFEKLMEWTSSNVMEERT |
| | | MKVYLPHMRMEEKYNLTSVLMALGVTDLFSSSANLSGISSAESLKMSEAVHEAF |
| | | VEIYESGSQVVGSTGAGTEVTSVSEEFRVDHPFLFLIKHNPTNSILFFGRCFSP |
| 36 | Ovalbumin-like | MGSIGAASVEFCFDVFKELKVQHVNENIFYSPLSIISALSMVYLGARENTKAQIDK |
| | [Falco | VVHFDKIAGFGEAIESQCVTSASIHSLKDMFTQITKPSDNYSLSFASRLYAEEAYSI |
| | peregrinus] | LPEYLQCVKELYKGGLETISFQTAADQARDLINSWVESQTNGMIKNILQPGAVDL |
| | | ETEMVLVNAIYFKGMWEKAFKDEDTQTVPFRMTEQESKPVQMMYQVGSFKVA |
| | | VMASDKIKILELPYASGQLSMVVVLPDDVSGLEQLEASITSEKLMEWTSSSIMEEK |
| | | KIKVYFPHMKIEEKYNLTSVLMALGMTDLFSSSANLSGISSAEKLKVSEAVHEAFV |
| | | EISEAGSEVVGSTEAGTEVTSVSEEFKADHPFLFLIKHNPTNSILFFGRCFSP |
| 37 | PREDICTED: | MGSIGAASSEFCFDIFKELKVQHVNENIFYSPLSIISALSMVYLGARENTRAQIDKV |
| | Ovalbumin -like | VPFDKITASGESIESQCSTSVSVHTSLKDIFTQITKSSDNHSLSFASRLYAEETYPILP |
| | isoform X2 | EYLQCVKELYEGGLETISFQTAADQARELINSWIESQTNGRIKNILQPGSVDPQTE |
| | [Phalacrocorax | MVLVNAIYFKGMWEKAFKDEDTQAVPFRMTEQESKPVQVMHQIGSFKVAVLAS |
| | carbo] | EKIKILELPYASGELSMLVLLPDDVSGLEQLETAITFEKLMEWTSPNIMEERKIKVF |
| | | LPRMKIEEKYNLTSVLMALGITDLFSPLANLSGISSAESLKMSEAIHEAFVEISEAGS |
| | | EVIGSTEAEVEVINDPEEFRADHPFLFLIKHNPTNSILFFGRCFSP |
| 38 | PREDICTED: | MGSIGAASTEFCFDVFKELKAQYVNENIFYSPMTIITALSMVYLGSKENTRAQIAK |
| | Ovalbumin-like | VAHFDKITGFGESIESQCGASASIQFSLKDLFTQITKPSGNHSLSVASRIYAEETYPI |
| | [Merops nubicus] | LPEYLECMKELYKGGLETINFQTAANQARELINSWVERQTSGMIKNILQPSSVDS |
| | | QTEMVLVNAIYFRGLWEKAFKVEDTQATPFRITEQESKPVQMMHQIGSFKVAVV |
| | | ASEKIKILELPYASGRLTMLVVLPDDVSGLKQLETTITFEKLMEWTTSNIMEERKI |
| | | KVYLPRMKIEEKYNLTSVLMALGLTDLFSSSANLSGISSAESLKMSEAVHEAFVEI |
| | | YEAGSEVVASAEAGMDATSVSEEFRADHPFLFLIKDNTSNSILFFGRCFSP |
| 39 | PREDICTED: | MGSIGAASTEFCFDVFKELKGQHVNENIFFCPLSIVSALSMVYLGARENTRAQIVK |
| | Ovalbumin-like | VAHFDKIAGFAESIESQCGTSVSIHTSLKDMFTQITKPSDNYSLNFASRLYAEETYP |
| | [Tauraco | IIPEYLQCVKELYKGGLETISFQTAADQAREIINSWVESQTNGMIKNILRPSSVHPQ |
| | erythrolophus] | TELVLVNAVYFKGTWEKAFKDEDTQAVPFRITEQESKPVQMMYQIGSFKVAAVT |
| | | SEKMKILEVPYASGELSMLVLLPDDVSGLEQLETAITAEKLIEWTSSTVMEERKLK |
| | | VYLPRMKIEEKYNLTTVLTALGVTDLFSSSANLSGISSAQGLKMSNAVHEAFVEIY |
| | | EAGSEVVGSKGEGTEVSSVSDEFKADHPFLFLIKHNPTNSIVFFGRCFSP |
| 40 | PREDICTED: | MGSIGAASTEFCFDVFKELKVHHVNENILYSPLAIISALSMVYLGAKENTRDQIDK |
| | Ovalbumin-like | VVHFDKITGIGESIESQCSTAVSVHTSLKDVFDQITRPSDNYSLAFASRLYAEKTYP |
| | [Cuculus | ILPEYLQCVKELYKGGLETIDFQTAADQARQLINSWVEDETNGMIKNILRPSSVNP |
| | canorus] | QTKIILVNAIYFKGMWEKAFKDEDTQEVPFRITEQETKSVQMMYQIGSFKVAEVV |
| | | SDKMKILELPYASGKLSMLVLLPDDVYGLEQLETVITVEKLKEWTSSIVMEERITK |
| | | VYLPRMKIMEKYNLTSVLTAFGITDLFSPSANLSGISSTESLKVSEAVHEAFVEIHE |
| | | AGSEVVGSAGAGIEATSVSEEFKADHPFLFLIKHNPTNSILFFGRCFSP |
| 41 | Ovalbumin | MGSIGAASTEFCLDVFKELKVQHVNENIFYSPLSIISALSMVYLGARENTRAQIDK |
| | [Antrostomus | VVHFDKITGFEDSIESQCGTSVSVHTSLKDMFTQITKPSDNYSVGFASRLYAAETY |
| | carolinensis] | QILPEYSQCVKELYKGGLETINFQKAADQATELINSWVESQTNGMIKNILQPSSVD |
| | | PQTQIFLVNAIYFKGMWQRAFKEEDTQAVPFRISEKESKPVQMMYQIGSFKVAVI |
| | | PSEKIKILELPYASGLLSMLVILPDDVSGLEQLENAITLEKLMQWTSSNMMEERKI |
| | | KVYLPRMRMEEKYNLTSVFMALGITDLFSSSANLSGISSAESLKMSDAVHEASVEI |
| | | HEAGSEVVGSTGSGTEASSVSEEFRADHPYLFLIKHNPTDSIVFFGRCFSP |
| 42 | PREDICTED: | MGSIGAASTEFCFDVFKELKFQHVDENIFYSPLTIISALSMVYLGARENTRAQIDK |
| | Ovalbumin-like | VVHFDKIAGFEETVESQCGTSVSVHTSLKDMFAQITKPSDNYSLSFASRLYAEETY |
| | [Opisthocomus | PILPEYLQCVKELYKGGLETISFQTAADQARDLINSWVESQTNGMIKNILQPSSVG |
| | hoazin] | PQTELILVNAIYFKGMWQKAFKDEDTQEVPFRMTEQQSKPVQMMYQTGSFKVA |
| | | VVASEKMKILALPYASGQLSLLVMLPDDVSGLKQLESAITSEKLIEWTSPSMMEE |
| | | RKIKVYLPRMKIEEKYNLTSVLMALGITDLFSPSANLSGISSAESLKMSQAVHEAF |
| | | VEIYEAGSEVVGSTGAGMEDSSDSEEFRVDHPFLFFIKHNPTNSILFFGRCFSP |
| 43 | PREDICTED: | MGSIGPLSVEFCCDVFKELRIQHPRENIFYSPVTIISALSMVYLGARDNTKAQIEKA |
| | Ovalbumin-like | VHFDKIPGFGESIESQCGTSLSIHTSLKDIFTQITKPSDNYTVGIASRLYAEEKYPILP |
| | [Lepidothrix | EYLQCIKELYKGGLEPINFQTAAEQARELINSWVESQTNGMIKNILQPSSVNPETD |
| | coronata] | MVLVNAIYFKGLWEKAFKDEDIQTVPFRITEQESKPVQMMFQIGSFRVAEITSEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAESLKVSSAFHEASVEIYEAGSKVV |
| | | GSTGAEVEDTSVSEEFRADHPFLFLIKHNPSNSIFFFGRCFSP |
| 44 | PREDICTED: | MGSIGTASAEFCFDVFKELKVHHVNENIFYSPLSIISALSMVYLGARENTKTQMEK |
| | Ovalbumin | VIHFDKITGLGESMESQCGTGVSIHTALKDMLSEITKPSDNYSLSLASRLYAEQTY |
| | [Struthio camelus | AILPEYLQCIKELYKESLETVSFQTAADQARELINSWIESQTNGVIKNFLQPGSVDS |
| | australis] | QTELVLVNAIYFKGMWEKAFKDEDTQEVPFRITEQESRPVQMMYQAGSFKVATV |
| | | AAEKIKILELPYASGELSMLVLLPDDISGLEQLETTISFEKLTEWTSSNMMEDRNM |
| | | KVYLPRMKIEEKYNLTSVLIALGMTDLFSPAANLSGISAAESLKMSEAIHAAYVEI |
| | | YEADSEIVSSAGVQVEVTSDSEEFRVDHPFLFLIKHNPTNSVLFFGRCISP |
| 45 | PREDICTED: | MGSIGAVSTEFSCDVFKELRIHHVQENIFYSPVTIISALSMIYLGARDSTKAQIEKA |
| | Ovalbumin-like | VHFDKIPGFGESIESQCGTSLSIHTSIKDMFTKITKASDNYSIGIASRLYAEEKYPILP |
| | [Acanthisitta | EYLQCVKELYKGGLESISFQTAAEQAREIINSWVESQTNGMIKNILQPSSVDPQTDI |
| | chloris] | VLVNAIYFKGLWEKAFRDEDTQTVPFKITEQESKPVQMMYQIGSFKVAEITSEKIK |
| | | ILEVPYASGQLSLWVLLPDDISGLEKLETAITFENLKEWTSSTKMEERKIKVYLPR |
| | | MKIEEKYNLTSVLTALGITDLFSSSANLSGISSAESLKVSEAFHEAIVEISEAGSKVV |
| | | GSVGAGVDDTSVSEEFRADHPFLFLIKHNPTSSIFFFGRCFSP |
| 46 | PREDICTED: | MGSIGAASTEFCFDVFKELKVQHVNENIFYSPLSIISALSMVYLGARENTRAQIDK |
| | Ovalbumin-like | VVHFDKIAGFGESTESQCGTSVSAHTSLKDMSNQITKLSDNYSLSFASRLYAEETY |
| | [Tyto alba] | PILPEYSQCVKELYKGGLESISFQTAAYQARELINAWVESQTNGMIKDILQPGSVD |
| | | SQTKMVLVNAIYFKGIWEKAFKDEDTQEVPFRMTEQETKPVQMMYQIGSFKVAV |
| | | IAAEKIKILELPYASGQLSMLVILPDDVSGLEQLETAITFEKLTEWTSASVMEERKI |
| | | KVYLPRMSIEEKYNLTSVLIALGVTDLFSSSANLSGISSAESLRMSEAIHEAFVETY |
| | | EAGSTESGTEVTSASEEFRVDHPFLFLIKHKPTNSILFFGRCFSP |
| 47 | PREDICTED: | MGSIGAASSEFCFDIFKELKVQHVNENIFYSPLSIISALSMVYLGARENTRAQIDKV |
| | Ovalbumin -like | VPFDKITASGESIESQVQKIQCSTSVSVHTSLKDIFTQITKSSDNHSLSFASRLYAEE |
| | isoform X1 | TYPILPEYLQCVKELYEGGLETISFQTAADQARELINSWIESQTNGRIKNILQPGSV |
| | [Phalacrocorax | DPQTEMVLVNAIYFKGMWEKAFKDEDTQAVPFRMTEQESKPVQVMHQIGSFKV |
| | carbo] | AVLASEKIKILELPYASGELSMLVLLPDDVSGLEQLETAITFEKLMEWTSPNIMEE |
| | | RKIKVFLPRMKIEEKYNLTSVLMALGITDLFSPLANLSGISSAESLKMSEAIHEAFV |
| | | EISEAGSEVIGSTEAEVEVINDPEEFRADHPFLFLIKHNPTNSILFFGRCFSP |
| 48 | Ovalbumin-like | MGSIGPLSVEFCCDVFKELRIQHARENIFYSPVTIISALSMVYLGARDNTKAQIEKA |
| | [Pipra filicauda] | VHFDKIPGFGESIESQCGTSLSIHTSLKDIFTQITKPSDNYTVGIASRLYAEEKYPILP |
| | | EYLQCIKELYKGGLEPISFQTAAEQARELINSWVESQINGIIKNILQPSSVNPETDM |
| | | VLVNAIYFKGLWEKAFKDEGTQTVPFRITEQESKPVQMMFQIGSFRVAEIASEKIR |
| | | ILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEINEAGSKV |
| | | VGAGVDDTSVSEEFRVDRPFLFLIKHNPSNSIFFFGRCFSP |
| 49 | Ovalbumin | MGSIGAASTEFCFDMFKELKVHHVNENIIYSPLSIISILSMVFLGARENTKTQMEKV |
| | [Dromaius | IHFDKITGFGESLESQCGTSVSVHASLKDILSEITKPSDNYSLSLASKLYAEETYPVL |
| | novaehollandiae] | PEYLQCIKELYKGSLETVSFQTAADQARELINSWVETQTNGVIKNFLQPGSVDPQT |
| | | EMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFP |
| 50 | Chain A, | MGSIGAASTEFCFDMFKELKVHHVNENIIYSPLSIISILSMVFLGARENTKTQMEKV |
| | Ovalbumin | IHFDKITGFGESLESQCGTSVSVHASLKDILSEITKPSDNYSLSLASKLYAEETYPVL |
| | | PEYLQCIKELYKGSLETVSFQTAADQARELINSWVETQTNGVIKNFLQPGSVDPQT |
| | | EMVLVDAIYFKGTWEKAFKDEDTQEVPFRITEQESKPVQMMYQAGSFKVATVA |
| | | AEKMKILELPYASGELSMFVLLPDDISGLEQLETTISIEKLSEWTSSNMMEDRKMK |
| | | VYLPHMKIEEKYNLTSVLVALGMTDLFSPSANLSGISTAQTLKMSEAIHGAYVEIY |
| | | EAGSEMATSTGVLVEAASVSEEFRVDHPFLFLIKHNPSNSILFFGRCIFPHHHHHH |
| 51 | Ovalbumin-like | MGSIGPLSVEFCCDVFKELRIQHARENIFYSPVTIISALSMVYLGARDNTKAQIEKA |
| | [Corapipo altera] | VHFDKIPGFGESIESQCGTSLSIHTSLKDIFTQITKPSDNYTVGIASRLYAEEKYPILP |
| | | EYLQCIKELYKGGLEPISFQTAAEQARELINSWVESQTNGMIKNILQPSAVNPETD |
| | | MVLVNAIYFKGLWEKAFKDEGTQTVPFRITEQESKPVQMMFQIGSFRVAEITSEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEIYEAGSKV |
| | | VGSTGAGVDDTSVSEEFRVDRPFLFLIKHNPSNSIFFFGRCFSP |
| 52 | Ovalbumin-like | MEDQRGNTGFTMGSIGAASTEFCIDVFRELRVQHVNENIFYSPLTIISALSMVYLG |
| | protein [Amazona | ARENTRAQIDQVVHFDKIAGFGDTVESQCGSSPSVHNSLKTVXAQITQPRDNYSL |
| | aestiva] | NLASRLYAEESYPILPEYLQCVKELYNGGLETVSFQTAADQARELINSWVESQTN |
| | | GIIKNILQPSSVDPQTEMVLVNAIYFKGLWEKAFKDEETQAVPFRITEQENRPVQM |
| | | MYQFGSFKVAXVASEKIKILELPYASGQLSMLVLLPDEVSGLEQNAITFEKLTEW |
| | | TSSDLMEERKIKVFFPRVKIEEKYNLTAVLVSLGITDLFSSSANLSGISSAENLKMS |
| | | EAVHEAXVEIYEAGSEVAGSSGAGIEVASDSEEFRVDHPFLFLIXHNPTNSILFFGR |
| | | CFSP |
| 53 | PREDICTED: | MGSIGAASTEFCIDVFRELRVQHVNENIFYSPLSIISALSMVYLGARENTRAQIDEV |
| | Ovalbumin-like | FHFDKIAGFGDTVDPQCGASLSVHKSLQNVFAQITQPKDNYSLNLASRLYAEESY |
| | [Melopsittacus | PILPEYLQCVKELYNEGLETVSFQTGADQARELINSWVENQTNGVIKNILQPSSVD |
| | undulatus] | PQTEMVLVNAIYFKGLWQKAFKDEETQAVPFRITEQENRPVQMMYQFGSFKVAV |
| | | VASEKVKILELPYASGQLSMWVLLPDEVSGLEQLENAITFEKLTEWTSSDLTEER |
| | | KIKVFLPRVKIEEKYNLTAVLMALGVTDLFSSSANFSGISAAENLKMSEAVHEAFV |
| | | EIYEAGSEVVGSSGAGIEAPSDSEEFRADHPFLFLIKHNPTNSILFFGRCFSP |
| 54 | Ovalbumin-like | MGSIGPLSVEFCCDVFKELRIQHARDNIFYSPVTIISALSMVYLGARDNTKAQIEKA |
| | [Neopelma | VHFDKIPGFGESIESQCGTSLSVHTSLKDIFTQITKPRENYTVGIASRLYAEEKYPIL |
| | chrysocephalum] | PEYLQCIKELYKGGLEPISFQTAAEQARELINSWVESQTNGMIKNILQPSSVNPETD |
| | | MVLVNAIYFKGLWKKAFKDEGTQTVPFRITEQESKPVQMMFQIGSFRVAEITSEKI |
| | | RILELPYASGQLSLWVLLPDDISGLEQLESAITFENLKEWTSSTKMEERKIKVYLPR |
| | | MKIEEKYNLTSVLTSLGITDLFSSSANLSGISSAEKLKVSSAFHEASMEIYEAGNKV |
| | | VGSTGAGVDDTSVSEEFRVDRPFLFLIKHNPSNSIFFFGRCFSP |
| 55 | PREDICTED: | MGSIGAASAEFCVDVFKELKDQHVNNIVFSPLMIISALSMVNIGAREDTRAQIDKV |
| | Ovalbumin-like | VHFDKITGYGESIESQCGTSIGIYFSLKDAFTQITKPSDNYSLSFASKLYAEETYPIL |
| | [Buceros | PEYLKCVKELYKGGLETISFQTAADQARELINSWVESQTNGMIKNILQPSSVDPQT |
| | rhinoceros | EMVLVNAIYFKGLWEKAFKDEDTQAVPFRITEQESKPVQMMYQIGSFKVAVIASE |
| | silvestris] | KIKILELPYASGQLSLLVLLPDDVSGLEQLESAITSEKLLEWTNPNIMEERKTKVYL |
| | | PRMKIEEKYNLTSVLVALGITDLFSSSANLSGISSAEGLKLSDAVHEAFVEIYEAGR |
| | | EVVGSSEAGVEDSSVSEEFKADRPFIFLIKHNPTNGILYFGRYISP |
| 56 | PREDICTED: | MGSIGAANTDFCFDVFKELKVHHANENIFYSPLSIVSALAMVYLGARENTRAQID |
| | Ovalbumin-like | KALHFDKILGFGETVESQCDTSVSVHTSLKDMLIQITKPSDNYSFSFASKIYTEETY |
| | [Cariama | PILPEYLQCVKELYKGGVETISFQTAADQAREVINSWVESHTNGMIKNILQPGSVD |
| | cristata] | PQTKMVLVNAVYFKGIWEKAFKEEDTQEMPFRINEQESKPVQMMYQIGSFKLTV |
| | | AASENLKILEFPYASGQLSMMVILPDEVSGLKQLETSITSEKLIKWTSSNTMEERKI |
| | | RVYLPRMKIEEKYNLKSVLMALGITDLFSSSANLSGISSAESLKMSEAVHEAFVEIY |
| | | EAGSEVTSSTGTEMEAENVSEEFKADHPFLFLIKHNPTDSIVFFGRCMSP |
| 57 | Ovalbumin | MGSIGPLSVEFCCDVFKELRIQHARENIFYSPVTIISALSMVYLGARDNTKAQIEKA |
| | [Manacus | VHFDKIPGFGESIESQCGTSLSIHTSLKDIFTQITKPSDNYTVGIASRLYAEEKYPILP |
| | vitellinus] | EYLQCIKELYKGGLEPISFQTAAEQARELINSWVESQTNGMIKNILQPSSVNPETD |
| | | MVLVNAIYFKGLWEKAFKDESTQTVPFRITEQESKPVQMMFQIGSFRVAEIASEKI |
| | | RILELPYASGQLSLWVLLPDDISGLEQLETAITFENLKEWTSSTKMEERKIKVYLPR |
| | | MKIEEKYNLTSVLTSLGITDLFSSSANLSGISSAERLKVSSAFHEASMEIYEAGSRV |
| | | VEAGVDDTSVSEEFRVDRPFLFLIKHNPSNSIFFFGRCFSP |
| 58 | Ovalbumin-like | MGSIGPVSTEFCCDIFKELRIQHARENIIYSPVTIISALSMVYLGARDNTKAQIEKAV |
| | [Empidonax | HFDKIPGFGESIESQCGTSLSIHTSLKDILTQITKPSDNYTVGIASRLYAEEKYPILSE |
| | traillii] | YLQCIKELYKGGLEPISFQTAAEQARELINSWVESQTNGMIKNILQPSSVNPETDM |
| | | VLVNAIYFKGLWEKAFKDEGTQTVPFRITEQESKPVQMMFQIGSFKVAEITSEKIR |
| | | ILELPYASGKLSLWVLLPDDISGLEQLETAITFENLKEWTSSTRMEERKIKVYLPR |
| | | MKIEEKYNLTSVLTSLGITDLFSSSANLSGISSAERLKVSSAFHEVFVEIYEAGSKVE |
| | | GSTGAGVDDTSVSEEFRADHPFLFLVKHNPSNSIIFFGRCYLP |
| 59 | PREDICTED: | MGSTGAASMEFCFALFRELKVQHVNENIFFSPVTIISALSMVYLGARENTRAQLD |
| | Ovalbumin-like | KVAPFDKITGFGETIGSQCSTSASSHTSLKDVFTQITKASDNYSLSFASRLYAEETY |
| | [Leptosomus | PILPEYLQCVKELYKGGLESISFQTAADQARELINSWVESQTNGMIKDILRPSSVDP |
| | discolor] | QTKIILITAIYFKGMWEKAFKEEDTQAVPFRMTEQESKPVQMMYQIGSFKVAVIPS |
| | | EKLKILELPYASGQLSMLVILPDDVSGLEQLETAITTEKLKEWTSPSMMKERKMK |
| | | VYFPRMRIEEKYNLTSVLMALGITDLFSPSANLSGISSAESLKVSEAVHEASVDIDE |
| | | AGSEVIGSTGVGTEVTSVSEEIRADHPFLFLIKHKPTNSILFFGRCFSP |
| 60 | Hypothetical | MEHAQLTQLVNSNMTSNTCHEADEFENIDFRMDSISVTNTKFCFDVFNEMKVHH |
| | protein | VNENILYSPLSILTALAMVYLGARGNTESQMKKALHFDSITGAGSTTDSQCGSSEY |
| | H355_008077 | IHNLFKEFLTEITRTNATYSLEIADKLYVDKTFTVLPEYINCARKFYTGGVEEVNFK |
| | [Colinus | TAAEEARQLINSWVEKETNGQIKDLLVPSSVDFGTMMVFINTIYFKGIWKTAFNTE |
| | virginianus] | DTREMPFSMTKQESKPVQMMCLNDTFNMATLPAEKMRILELPYASGELSMLVLL |
| | | PDEVSGLEQIEKAINFEKLREWTSTNAMEKKSMKVYLPRMKIEEKYNLTSTLMAL |
| | | GMTDLFSRSANLTGISSVENLMISDAVHGAFMEVNEEGTEAAGSTGAIGNIKHSVE |
| | | FEEFRADHPFLFLIRYNPTNVILFFDNSEFTMGSIGAVSTEFCFDVFKELRVHHANE |
| | | NIFYSPFTVISALAMVYLGAKDSTRTQINKVVRFDKLPGFGDSIEAQCGTSANVHS |
| | | SLRDILNQITKPNDIYSFSLASRLYADETYTILPEYLQCVKELYRGGLESINFQTAAD |
| | | QARELINSWVESQTSGIIRNVLQPSSVDSQTAMVLVNAIYFKGLWEKGFKDEDTQ |
| | | AMPFRVTEQENKSVQMMYQIGTFKVASVASEKMKILELPFASGTMSMWVLLPDE |
| | | VSGLEQLETTISIEKLTEWTSSSVMEERKIKVFLPRMKMEEKYNLTSVLMAMGMT |
| | | DLFSSSANLSGISSTLQKKGFRSQELGDKYAKPMLESPALTPQVTAWDNSWIVAH |
| | | PAAIEPDLCYQIMEQKWKPFDWPDFRLPMRVSCRFRTMEALNKANTSFALDFFKH |
| | | ECQEDDDENILFSPFSISSALATVYLGAKGNTADQMAKTEIGKSGNIHAGFKALDL |
| | | EINQPTKNYLLNSVNQLYGEKSLPFSKEYLQLAKKYYSAEPQSVDFLGKANEIRRE |
| | | INSRVEHQTEGKIKNLLPPGSIDSLTRLVLVNALYFKGNWATKFEAED |
| | | TRHRPFRINMHTTKQVPMMYLRDKFNWTYVESVQTDVLELPYVNNDLSMFILLP |
| | | RDITGLQKLINELTFEKLSAWTSPELMEKMKMEVYLPRFTVEKKYDMKSTLSKM |
| | | GIEDAFTKVDSCGVTNVDEITTHIVSSKCLELKHIQINKKLKCNKAVAMEQVSASI |
| | | GNFTIDLFNKLNETSRDKNIFFSPWSVSSALALTSLAAKGNTAREMAEDPENEQAE |
| | | NIHSGFKELMTALNKPRNTYSLKSANRIYVEKNYPLLPTYIQLSKKYYKAEPYKV |
| | | NFKTAPEQSRKEINNWVEKQTERKIKNFLSSDDVKNSTKSILVNAIYFKAEWEEKF |
| | | QAGNTDMQPFRMSKNKSKLVKMMYMRHTFPVLIMEKLNFKMIELPYVKRELSM |
| | | FILLPDDIKDSTTGLEQLERELTYEKLSEWADSKKMSVTLVDLHLPKFSMEDRYDL |
| | | KDALKSMGMASAFNSNADFSGMTGFQAVPMESLSASTNSFTLDLYKKLDETSKG |
| | | QNIFFASWSIATALAMVHLGAKGDTATQVAKGPEYEETENIHSGFKELLSAINKPR |
| | | NTYLMKSANRLFGDKTYPLLPKFLELVARYYQAKPQAVNFKTDAEQARAQINSW |
| | | VENETESKIQNLLPAGSIDSHTVLVLVNAIYFKGNWEKRFLEKDTSKMPFRLSKTE |
| | | TKPVQMMFLKDTFLIHHERTMKFKIIELPYVGNELSAFVLLPDDISDNTTGLELVE |
| | | RELTYEKLAEWSNSASMMKAKVELYLPKLKMEENYDLKSVLSDMGIRSAFDPAQ |
| | | ADFTRMSEKKDLFISKVIHKAFVEVNEEDRIVQLASGRLTGRCRTLANKELSEKNR |
| | | TKNLFFSPFSISSALSMILLGSKGNTEAQIAKVLSLSKAEDAHNGYQSLLSEINNPDT |
| | | KYILRTANRLYGEKTFEFLSSFIDSSQKFYHAGLEQTDFKNASEDSRKQINGWVEE |
| | | KTEGKIQKLLSEGIINSMTKLVLVNAIYFKGNWQEKFDKETTKEMPFKINKNETKP |
| | | VQMMFRKGKYNMTYIGDLETTVLEIPYVDNELSMIILLPDSIQDESTGLEKLEREL |
| | | TYEKLMDWINPNMMDSTEVRVSLPRFKLEENYELKPTLSTMGMPDAFDLRTADF |
| | | SGISSGNELVLSEVVHKSFVEVNEEGTEAAAATAGIMLLRCAMIVANFTADHPFLF |
| | | FIRHNKTNSILFCGRFCSP |
| 61 | PREDICTED: | MGSIGTASTEFCFDMFKEMKVQHANQNIIFSPLTIISALSMVYLGARDNTKAQMEK |
| | Ovalbumin | VIHFDKITGFGESVESQCGTSVSIHTSLKDMLSEITKPSDNYSLSLASRLYAEETYPI |
| | isoform X2 | LPEYLQCMKELYKGGLETVSFQTAADQARELINSWVESQTNGVIKNFLQPGSVDP |
| | [Apteryx australis | QTEMVLVNAIYFKGMWEKAFKDEDTQEVPFRITEQESKPVQMMYQVGSFKVAT |
| | mantelli] | VAAEKMKILEIPYTHRELSMFVLLPDDISGLEQLETTISFEKLTEWTSSNMMEERK |
| | | VKVYLPHMKIEEKYNLTSVLMALGMTDLFSPSANLSGISTAQTLMMSEAIHGAYV |
| | | EIYEAGREMASSTGVQVEVTSVLEEVRADKPFLFFIRHNPTNSMVVFGRYMSP |
| 62 | Hypothetical | MTSNTCHEADEFENIDFRMDSISVTNTKFCFDVFNEMKVHHVNENILYSPLSILTA |
| | protein | LAMVYLGARGNTESQMKKALHFDSITGGGSTTDSQCGSSEYIHNLFKEFLTEITRT |
| | ASZ78_006007 | NATYSLEIADKLYVDKTFTVLPEYINCARKFYTGGVEEVNFKTAAEEARQLMNS |
| | [Callipepla | WVEKETNGQIKDLLVPSSVDFGTMMVFINTIYFKGIWKTAFNTEDTREMPFSMTK |
| | squamata] | QESKPVQMMCLNDTFNMVTLPAEKMRILELPYASGELSMLVLLPDEVSGLERIEK |
| | | AINFEKLREWTSTNAMEKKSMKVYLPRMKIEEKYNLTSTLMALGMTDLFSRSAN |
| | | LTGISSVDNLMISDAVHGAFMEVNEEGTEAAGSTGAIGNIKHSVEFEEFRADHPFL |
| | | FLIRYNPTNVILFFDNSEFTMGSIGAVSTEFCFDVFKELRVHHANENIFYSPFTIISAL |
| | | AMVYLGAKDSTRTQINKVVRFDKLPGFGDSIEAQCGTSANVHSSLRDILNQITKPN |
| | | DIYSFSLASRLYADETYTILPEYLQCVKELYRGGLESINFQTAADQARELINSWVES |
| | | QTSGIIRNVLQPSSVDSQTAMVLVNAIYFKGLWEKGFKDEDTQAIPFRVTEQENKS |
| | | VQMMYQIGTFKVASVASEKMKILELPFASGTMSMWVLLPDEVSGLEQLETTISIE |
| | | KLTEWTSSSVMEERKIKVFLPRMKMEEKYNLTSVLMAMGMTDLFSSSANLSGISS |
| | | TLQKKGFRSQELGDKYAKPMLESPALTPQATAWDNSWIVAHPPAIEPDLYYQIME |
| | | QKWKPFDWPDFRLPMRVSCRFRTMEALNKANTSFALDFFKHECQEDDSENILFSP |
| | | FSISSALATVYLGAKGNTADQMAKVLHFNEAEGARNVTTTIRMQVYSRTDQQRL |
| | | NRRACFQKTEIGKSGNIHAGFKGLNLEINQPTKNYLLNSVNQLYGEKSLPFSKEYL |
| | | QLAKKYYSAEPQSVDFVGTANEIRREINSRVEHQTEGKIKNLLPPGSIDSLTRLVLV |
| | | NALYFKGNWATKFEAEDTRHRPFRINTHTTKQVPMMYLSDKFNWTYVESVQTD |
| | | VLELPYVNNDLSMFILLPRDITGLQKLINELTFEKLSAWTSPELMEKMKMEVYLPR |
| | | FTVEKKYDMKSTLSKMGIEDAFTKVDNCGVTNVDEITIHVVPSKCLELKHIQINKE |
| | | LKCNKAVAMEQVSASIGNFTIDLFNKLNETSRDKNIFFSPWSVSSALALTSLAAKG |
| | | NTAREMAEDPENEQAENIHSGFNELLTALNKPRNTYSLKSANRIYVEKNYPLLPTY |
| | | IQLSKKYYKAEPHKVNFKTAPEQSRKEINNWVEKQTERKIKNFLSSDDVKNSTKLI |
| | | LVNAIYFKAEWEEKFQAGNTDMQPFRMSKNKSKLVKMMYMRHTFPVLIMEKLN |
| | | FKMIELPYVKRELSMFILLPDDIKDSTTGLEQLERELTYEKLSEWADSKKMSVTLV |
| | | DLHLPKFSMEDRYDLKDALRSMGMASAFNSNADFSGMTGERDLVISKVCHQSFV |
| | | AVDEKGTEAAAATAVIAEAVPMESLSASTNSFTLDLYKKLDETSKGQNIFFASWSI |
| | | ATALTMVHLGAKGDTATQVAKGPEYEETENIHSGFKELLSALNKPRNTYSMKSA |
| | | NRLFGDKTYPLLPTKTKPVQMMFLKDTFLIHHERTMKFKIIELPYMGNELSAFVLL |
| | | PDDISDNTTGLELVERELTYEKLAEWSNSASMMKVKVELYLPKLKMEENYDLKS |
| | | ALSDMGIRSAFDPAQADFTRMSEKKDLFISKVIHKAFVEVNEEDRIVQLASGRLTG |
| | | NTEAQIAKVLSLSKAEDAHNGYQSLLSEINNPDTKYILRTANRLYGEKTFEFLSSFI |
| | | DSSQKFYHAGLEQTDFKNASEDSRKQINGWVEEKTEGKIQKLLSEGIINSMTKLVL |
| | | VNAIYFKGNWQEKFDKETTKEMPFKINKNETKPVQMMFRKGKYNMTYIGDLETT |
| | | VLEIPYVDNELSMIILLPDSIQDESTGLEKLERELTYEKLMDWINPNMMDSTEVRV |
| | | SLPRFKLEENYELKPTLSTMGMPDAFDLRTADFSGISSGNELVLSEVVHKSFVEVN |
| | | EEGTEAAAATAGIMLLRCAMIVANFTADHPFLFFIRHNKTNSILFCGRFCSP |
| 63 | PREDICTED: | MASIGAASTEFCFDVFKELKTQHVKENIFYSPMAIISALSMVYIGARENTRAEIDK |
| | Ovalbumin-like | VVHFDKITGFGNAVESQCGPSVSVHSSLKDLITQISKRSDNYSLSYASRIYAEETYP |
| | [Mesitornis | ILPEYLQCVKEVYKGGLESISFQTAADQARENINAWVESQTNGMIKNILQPSSVNP |
| | unicolor] | QTEMVLVNAIYLKGMWEKAFKDEDTQTMPFRVTQQESKPVQMMYQIGSFKVAV |
| | | IASEKMKILELPYTSGQLSMLVLLPDDVSGLEQVESAITAEKLMEWTSPSIMEERT |
| | | MKVYLPRMKMVEKYNLTSVLMALGMTDLFTSVANLSGISSAQGLKMSQAIHEAF |
| | | VEIYEAGSEAVGSTGVGMEITSVSEEFKADLSFLFLIRHNPTNSIIFFGRCISP |
| 64 | Ovalbumin, | MGSIGAASTEFCFDVFRELRVQHVNENIFYSPFSIISALAMVYLGARDNTRTQIDKI |
| | partial [Anas | SQFQALSDEHLVLCIQQLGEFFVCTNRERREVTRYSEQTEDKTQDQNTGQIHKIV |
| | platyrhynchos] | DTCMLRQDILTQITKPSDNFSLSFASRLYAEETYAILPEYLQCVKELYKGGLESISF |
| | | QTAADQARELINSWVESQTNGIIKNILQPSSVDSQTTMVLVNAIYFKGMWEKAFK |
| | | DEDTQAMPFRMTEQESKPVQMMYQVGSFKVAMVTSEKMKILELPFASGMMSMF |
| | | VLLPDEVSGLEQLESTISFEKLTEWTSSTMMEERRMKVYLPRMKMEEKYNLTSVF |
| | | MALGMTDLFSSSANMSGISSTVSLKMSEAVHAACVEIFEAGRDVVGSAEAGMDV |
| | | TSVSEEFRADHPFLFFIKHNPTNSILFFGRWMSP |
| 65 | PREDICTED: | MGSIGAASAEFCLDIFKELKVQHVNENIIFSPMTIISALSLVYLGAKEDTRAQIEKV |
| | Ovalbumin-like | VPFDKIPGFGEIVESQCPKSASVHSSIQDIFNQIIKRSDNYSLSLASRLYAEESYPIRP |
| | [Chaetura | EYLQCVKELDKEGLETISFQTAADQARQLINSWVESQTNGMIKNILQPSSVNSQTE |
| | pelagica] | MVLVNAIYFRGLWQKAFKDEDTQAVPFRITEQESKPVQMMQQIGSFKVAEIASE |
| | | KMKILELPYASGQLSMLVLLPDDVSGLEKLESSITVEKLIEWTSSNLTEERNVKVY |
| | | LPRLKIEEKYNLTSVLAALGITDLFSSSANLSGISTAESLKLSRAVHESFVEIQEAGH |
| | | EVEGPKEAGIEVTSALDEFRVDRPFLFVTKHNPTNSILFLGRCLSP |
| 66 | PREDICTED: | MGSISAASGEFCLDIFKELKVQHVNENIFYSPMVIVSALSLVYLGARENTRAQIDK |
| | Ovalbumin-like | VIPFDKITGSSEAVESQCGTPVGAHISLKDVFAQIAKRSDNYSLSFVNRLYAEETYP |
| | [Apaloderma | ILPEYLQCVKELYKGGLETISFQTAADQAREIINSWVESQTDGKIKNILQPSSVDPQ |
| | vittatum] | TKMVLVSAIYFKGLWEKSFKDEDTQAVPFRVTEQESKPVQMMYQIGSFKVAAIA |
| | | AEKIKILELPYASEQLSMLVLLPDDVSGLEQLEKKISYEKLTEWTSSSVMEEKKIK |
| | | VYLPRMKIEEKYNLTSILMSLGITDLFSSSANLSGISSTKSLKMSEAVHEASVEIYEA |
| | | GSEASGITGDGMEATSVFGEFKVDHPFLFMIKHKPTNSILFFGRCISP |
| 67 | Ovalbumin-like | MGSIGPVSTEVCCDIFRELRSQSVQENVCYSPLLIISTLSMVYIGAKDNTKAQIEKA |
| | [Corvus cornix | IHFDKIPGFGESTESQCGTSVSIHTSLKDIFTQITKPSDNYSISIARRLYAEEKYPILPE |
| | cornix] | YIQCVKELYKGGLESISFQTAAEKSRELINSWVESQTNGTIKNILQPSSVSSQTDMV |
| | | LVSAIYFKGLWEKAFKEEDTQTIPFRITEQESKPVQMMSQIGTFKVAEIPSEKCRIL |
| | | ELPYASGRLSLWVLLPDDISGLEQLETAITFENLKEWTSSSKMEERKIRVYLPRMK |
| | | IEEKYNLTSVLKSLGITDLFSSSANLSGISSAESLKVSAAFHEASVEIYEAGSKGVGS |
| | | SEAGVDGTSVSEEIRADHPFLFLIKHNPSDSILFFGRCFSP |
| 68 | PREDICTED: | MGSIGAASTEFCFDVFKELKVQHVNENIIISPLSIISALSMVYLGAREDTRAQIDKV |
| | Ovalbumin-like | VHFDKITGFGEAIESQCPTSESVHASLKETFSQLTKPSDNYSLAFASRLYAEETYPI |
| | [Calypte anna] | LPEYLQCVKELYKGGLETINFQTAAEQARQVINSWVESQTDGMIKSLLQPSSVDP |
| | | QTEMILVNAIYFRGLWERAFKDEDTQELPFRITEQESKPVQMMSQIGSFKVAVVA |
| | | SEKVKILELPYASGQLSMLVLLPDDVSGLEQLESSITVEKLIEWISSNTKEERNIKV |
| | | YLPRMKIEEKYNLTSVLVALGITDLFSSSANLSGISSAESLKISEAVHEAFVEIQEAG |
| | | SEVVGSPGPEVEVTSVSEEWKADRPFLFLIKHNPTNSILFFGRYISP |
| 69 | PREDICTED: | MGSIGPVSTEVCCDIFRELRSQSVQENVCYSPLLIISTLSMVYIGAKDNTKAQIEKA |
| | Ovalbumin | IHFDKIPGFGESTESQCGTSVSIHTSLKDIFTQITKPSDNYSISIARRLYAEEKYPILQ |
| | [Corvus | EYIQCVKELYKGGLESISFQTAAEKSRELINSWVESQTNGTIKNILQPSSVSSQTDM |
| | brachyrhynchos] | VLVSAIYFKGLWEKAFKEEDTQTIPFRITEQESKPVQMMSQIGTFKVAEIPSEKCRI |
| | | LELPYASGRLSLWVLLPDDISGLEQLETSITFENLKEWTSSSKMEERKIRVYLPRM |
| | | KIEEKYNLTSVLKSLGITDLFSSSANLSGISSAESLKVSAVFHEASVEIYEAGSKGVG |
| | | SSEAGVDGTSVSEEIRADHPFLFLIKHNPSDSILFFGRCFSP |
| 70 | Hypothetical | MLNLMHPKQFCCTMGSIGPVSTEVCCDIFRELRSQSVQENVCYSPLLIISTLSMVYI |
| | protein | GAKDNTKAQIEKAIHFDKIPGFGESTESQCGTSVSIHTSLKDIFTQITKPSDNYSISIA |
| | DUI87_08270 | SRLYAEEKYPILPEYIQCVKELYKGGLESISFQTAAEKSRELINSWVESQTNGTIKN |
| | [Hirundo rustica | ILQPSSVSSQTDMVLVSAIYFKGLWEKAFKEEDTQTVPFRITEQESKPVQMMSQIG |
| | rustica] | TFKVAEIPSEKCRILELPYASGRLSLWVLLPDDISGLEQLETAITSENLKEWTSSSK |
| | | MEERKIKVYLPRMKIEEKYNLTSVLKSLGITDLFSSSANLSGISSAESLKVSGAFHE |
| | | AFVEIYEAGSKAVGSSGAGVEDTSVSEEIRADHPFLFFIKHNPSDSILFFGRCFSP |
| 71 | Ostrich OVA | EAEAGSIGTASAEFCFDVFKELKVHHVNENIFYSPLSIISALSMVYLGARENTKTQ |
| | sequence as | MEKVIHFDKITGLGESMESQCGTGVSIHTALKDMLSEITKPSDNYSLSLASRLYAE |
| | secreted from | QTYAILPEYLQCIKELYKESLETVSFQTAADQARELINSWIESQTNGVIKNFLQPGS |
| | pichia | VDSQTELVLVNAIYFKGMWEKAFKDEDTQEVPFRITEQESRPVQMMYQAGSFKV |
| | | ATVAAEKIKILELPYASGELSMLVLLPDDISGLEQLETTISFEKLTEWTSSNMMED |
| | | RNMKVYLPRMKIEEKYNLTSVLIALGMTDLFSPAANLSGISAAESLKMSEAIHAA |
| | | YVEIYEADSEIVSSAGVQVEVTSDSEEFRVDHPFLFLIKHNPTNSVLFFGRCISP |
| 72 | Ostrich construct | MRFPSIFTAVLFAASSALAAPVNTTTEDETAQIPAEAVIGYSDLEGDFDVAVLPFS |
| | (secretion signal + | NSTNNGLLFINTTIASIAAKEEGVSLEKREAEAGSIGTASAEFCFDVFKELKVHHV |
| | mature protein) | NENIFYSPLSIISALSMVYLGARENTKTQMEKVIHFDKITGLGESMESQCGTGVSIH |
| | | TALKDMLSEITKPSDNYSLSLASRLYAEQTYAILPEYLQCIKELYKESLETVSFQTA |
| | | ADQARELINSWIESQTNGVIKNFLQPGSVDSQTELVLVNAIYFKGMWEKAFKDED |
| | | TQEVPFRITEQESRPVQMMYQAGSFKVATVAAEKIKILELPYASGELSMLVLLPD |
| | | DISGLEQLETTISFEKLTEWTSSNMMEDRNMKVYLPRMKIEEKYNLTSVLIALGM |
| | | TDLFSPAANLSGISAAESLKMSEAIHAAYVEIYEADSEIVSSAGVQVEVTSDSEEFR |
| | | VDHPFLFLIKHNPTNSVLFFGRCISP |
| 73 | Duck OVA | EAEAGSIGAASTEFCFDVFRELRVQHVNENIFYSPFSIISALAMVYLGARDNTRTQI |
| | sequence as | DKVVHFDKLPGFGESMEAQCGTSVSVHSSLRDILTQITKPSDNFSLSFASRLYAEE |
| | secreted from | TYAILPEYLQCVKELYKGGLESISFQTAADQARELINSWVESQINGIIKNILQPSSV |
| | pichia | DSQTTMVLVNAIYFKGMWEKAFKDEDTQAMPFRMTEQESKPVQMMYQVGSFK |
| | | VAMVTSEKMKILELPFASGMMSMFVLLPDEVSGLEQLESTISFEKLTEWTSSTMM |
| | | EERRMKVYLPRMKMEEKYNLTSVFMALGMTDLFSSSANMSGISSTVSLKMSEAV |
| | | HAACVEIFEAGRDVVGSAEAGMDVTSVSEEFRADHPFLFFIKHNPTNSILFFGRWM |
| | | SP |
| 74 | Duck construct | MRFPSIFTAVLFAASSALAAPVNTTTEDETAQIPAEAVIGYSDLEGDFDVAVLPFSN |
| | (secretion signal + | STNNGLLFINTTIASIAAKEEGVSLEKREAEAGSIGAASTEFCFDVFRELRVQHVNE |
| | mature protein) | NIFYSPFSIISALAMVYLGARDNTRTQIDKVVHFDKLPGFGESMEAQCGTSVSVH |
| | | SSLRDILTQITKPSDNFSLSFASRLYAEETYAILPEYLQCVKELYKGGLESISFQTAA |
| | | DQARELINSWVESQTNGIIKNILQPSSVDSQTTMVLVNAIYFKGMWEKAFKDEDT |
| | | QAMPFRMTEQESKPVQMMYQVGSFKVAMVTSEKMKILELPFASGMMSMFVLLP |
| | | DEVSGLEQLESTISFEKLTEWTSSTMMEERRMKVYLPRMKMEEKYNLTSVFMAL |
| | | GMTDLFSSSANMSGISSTVSLKMSEAVHAACVEIFEAGRDVVGSAEAGMDVTSVS |
| | | EEFRADHPFLFFIKHNPTNSILFFGRWMSP |

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method for increasing recovery and purity of a secreted protein, the method comprising steps of:
obtaining recombinant fungal cells capable of expressing a secreted protein;
culturing the recombinant fungal cells under conditions that promote expression and secretion of the recombinant protein into a culturing medium;
introducing ammonium sulfate to the culturing medium to achieve an ammonium sulfate concentration above 200 g/l, thereby precipitating the secreted protein;
recovering the precipitated protein; and
diafiltering and/or ultrafiltering the precipitated secreted protein.

2. The method of claim 1, wherein the method further comprises a step of centrifuging the culturing medium to remove the recombinant fungal cells and other cellular components prior to introducing ammonium sulfate, wherein the centrifuged culturing medium is not microfiltered prior to introducing ammonium sulfate.

3. (canceled)

4. The method of claim 1, wherein the method comprises a step of microfiltering the diafiltered and/or ultrafiltered protein of interest, wherein the microfiltering comprises a filter capable of capturing fungal cells and other cellular components.

5-6. (canceled)

7. The method of claim 1, wherein the method further comprises adding an agent to maintain the pH at about 4.5 to about 6.5 after centrifuging the culturing medium, wherein the agent is an acid or a base.

8-11. (canceled)

12. The method of claim 7, wherein adding the agent occurs before introducing the ammonium sulfate; wherein adding the agent occurs after introducing the ammonium sulfate; or wherein adding the agent is contemporaneous with introducing the ammonium sulfate.

13-14. (canceled)

15. The method of claim 1, wherein the method comprises recovering the precipitated protein in a medium having a pH of about 4.5-6.5.

16-18. (canceled)

19. The method of claim 1, wherein the method further comprises a step of drying the further microfiltered protein, thereby obtaining a dried protein product.

20. The method of claim 1, wherein the method further comprises a step of solubilizing the precipitated secreted protein with water to obtain a solubilized protein prior to ultrafiltering the precipitated secreted protein of interest.

21. The method of claim 1, wherein the ammonium sulfate concentration is about or above 200 g/l, the ammonium sulfate concentration is about or above 300 g/l, or the ammonium sulfate concentration is about or above 400 g/l.

22. (canceled)

23. The method of claim 1, wherein the amount of recovered protein of interest is greater than the recovery that is obtained from a method that does not comprise adding ammonium sulfate to the culturing medium to achieve an ammonium sulfate concentration above 200 g/l.

24. The method of claim 1, wherein recovery of the protein is at least about 40% w/w, is at least about 45% w/w, is at least about 50% w/w, is at least about 55% w/w, is at least about 60% w/w for the final dried product, is at least about 65% w/w for the final dried product, is at least about 70% w/w for the final dried product, is at least about 75% w/w for the final dried product, is at least about 80% w/w for the final dried product, is at least about 85% w/w for the final dried product, or is at least about 90% w/w for the final dried product, wherein the recovery is: the weight of the protein of interest recovered in the final product relative to the weight of the protein of interest prior to introducing ammonium sulfate or the weight of the protein recovered in the final product relative to the sum of weight of the protein recovered and the weight of the protein remaining in the supernatant following introducing ammonium sulfate.

25. (canceled)

26. The method of claim 1, wherein the method does not comprise use of a purification resin and/or a purification column.

27. The method of claim 1, wherein the fungal cells are of the species selected from Agaricus bisporus; Agaricus spp.; Aspergillus awamori; Aspergillus fumigatus; Aspergillus nidulans; Aspergillus niger; Aspergillus oryzae; Aspergillus oryzae; Aspergillus spp.; Colletotrichum gloeosporiodes; Colletotrichum spp.; Endothia parasitica; Endothia spp.; Fusarium graminearum; Fusarium solani; Fusarium spp.; Komagatella pastoris; Komagatella phaffi; Mucor miehei; Mucor pusillus; Mucor spp.; Myceliophthora spp.; Myceliophthora thermophila; Neurospora crassa; Neurospora spp.; Penicillium (Talaromyces) emersonii; Penicillium camemberti; Penicillium canescens; Penicillium chrysogenum; Penicillium funiculosum; Penicillium purpurogenum; Penicillium roqueforti; Penicillium spp.; Pichia angusta; Pichia pastoris; Pichia pastoris; Pichia Pastoris “MutS” strain; Pichia spp.; Pleurotus ostreatus; Pleurotus spp.; Rhizomucor miehei; Rhizomucor pusillus; Rhizomucor spp.; Rhizopus arrhizus; Rhizopus oligosporus; Rhizopus oryzae; Rhizopus spp.; Trichoderma altroviride; Trichoderma reesei; Trichoderma spp.; Trichoderma vireus; Yarrowia lipolytica; and Yarrowia spp.

28-30. (canceled)

31. The method of claim 1, wherein the protein is a food protein, wherein the food protein is used as nutritional, dietary, digestive, supplements, such as in food products and feed products; and wherein the food protein is selected from a plant protein and an animal protein.

32-34. (canceled)

35. The method of claim 31, wherein the animal protein is an egg white protein selected from ovalbumin, ovomucoid, ovotransferrin, lysozyme, ovomucin, ovoglobulin G2, ovoglobulin G3, ovoinhibitor, ovoglycoprotein, flavoprotein, ovomacroglobulin, ovostatin, cystatin, avidin, ovalbumin related protein X, ovalbumin related protein Y, and any combination thereof.

36. (canceled)

37. The method of claim 35, wherein the egg white protein is an ovalbumin (OVA) that comprises the amino acid sequence of a chicken OVA, a goose OVA, a quail OVA, an ostrich OVA, or a duck OVA, and wherein the egg white protein has at least 70%, 75%, 80%, 85%, 90%, 95%, 96%, 97%, 98%, or 99% sequence identity to one of SEQ ID NO: 1 to SEQ ID NO: 74.

38. (canceled)

39. The method of claim 35, wherein the egg white protein is a recovered recombinant OVA (rOVA); wherein a glycosylation pattern of the rOVA is devoid of N-linked galactose units.

40-41. (canceled)

42. The method of claim 39, wherein the rOVA is a glycosylated or a non-glycosylated rOVA, and wherein the amino acid sequence of the rOVA lacks an N-terminal methionine.

43. (canceled)

44. The method of claim 1, wherein the recovered protein is suitable for use in a food product, wherein the food product has an additional characteristic equivalent to or better than a similar baked item made with a natural egg white or a natural whole egg, wherein the characteristic is selected from the group consisting of foam capacity, foam stability, hardness, chewiness, guminess, and springineness.

45-47. (canceled)

48. A powdered composition comprising the recovered protein of claim 1.

49. A liquid composition comprising the powdered composition of claim 48 and a solvent suitable for animal or human consumption.

50. A method for increasing recovery and purity of a secreted protein, the method comprising steps of:
obtaining recombinant fungal cells capable of expressing a secreted protein;
culturing the recombinant fungal cells under conditions that promote expression and secretion of the recombinant protein into a culturing medium;
centrifuging the culturing medium to remove the recombinant fungal cells and other cellular components;
introducing ammonium sulfate to the culturing medium to achieve an ammonium sulfate concentration above 200 g/l, thereby precipitating the secreted protein, wherein the centrifuged culturing medium is not microfiltered prior to introducing the ammonium sulfate;
recovering the precipitated protein;
solubilizing the precipitated secreted protein with water to obtain a solubilized protein;
diafiltering and/or ultrafiltering the solubilized protein;
microfiltering the diafiltered and/or ultrafiltered protein; and
drying the further microfiltered protein, thereby obtaining a dried protein product.

51. A baked food item comprising a recovered and purified secreted protein of claim 1, wherein the baked food item has an additional characteristic equivalent to or better than a similar baked item made with a natural egg white or a natural whole egg, wherein the characteristic is selected from the group consisting of foam capacity, foam stability, hardness, chewiness, guminess, and springineness.

52-53. (canceled)

54. A non-meat food item comprising a recovered and purified secreted protein of claim 1, wherein the non-meat food item has an additional characteristic equivalent to or better than a similar non-meat item made with a natural egg white or a natural whole egg, wherein the characteristic is selected from the group consisting of foam capacity, foam stability, hardness, chewiness, guminess, springineness, less oily and retains more moisture.

55-56. (canceled)


SEQUENCE LISTING (74 sequences)

SEQ ID NO: 1 - protein, 474 aa, Gallus gallus
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 389 aa, synthetic construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 3 - protein, 419 aa, Achromobacter denitrificans
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 4 - protein, 406 aa, synthetic construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 5 - protein, 451 aa, Achromobacter denitrificans
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 6 - protein, 386 aa, Meleagris gallopavo
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 7 - protein, 386 aa, Meleagris gallopavo
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 8 - protein, 417 aa, Bambusicola thoracicus
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 9 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 10 - protein, 386 aa, Numida meleagris
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 11 - protein, 411 aa, Numida meleagris
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 12 - protein, 383 aa, Coturnix japonica
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 13 - protein, 408 aa, Coturnix japonica
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 14 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 15 - protein, 386 aa, Anas platyrhynchos
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 16 - protein, 386 aa, Anser cygnoides
  source 1..386: note = Anser cygnoides domesticus
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 17 - protein, 386 aa, Aquila chrysaetos
  source 1..386: note = Aquila chrysaetos canadensis
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 18 - protein, 386 aa, Haliaeetus albicilla
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 19 - protein, 386 aa, Haliaeetus leucocephalus
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 20 - protein, 386 aa, Fulmarus glacialis
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 21 - protein, 386 aa, Chlamydotis macqueenii
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 22 - protein, 386 aa, Nipponia nippon
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 23 - protein, 386 aa, Gavia stellata
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 24 - protein, 386 aa, Pelecanus crispus
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 25 - protein, 386 aa, Charadrius vociferus
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 26 - protein, 386 aa, Eurypyga helias
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 27 - protein, 391 aa, Gavia stellata
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 28 - protein, 386 aa, Egretta garzetta
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 29 - protein, 386 aa, Balearica regulorum
  source 1..386: note = Balearica regulorum gibbericeps
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 30 - protein, 386 aa, Nestor notabilis
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 31 - protein, 386 aa, Pygoscelis adeliae
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 32 - protein, 385 aa, Athene cunicularia
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 33 - protein, 386 aa, Calidris sp.
  source 1..386: note = Calidris pugnax
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 34 - protein, 386 aa, Aptenodytes forsteri
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 35 - protein, 386 aa, Pterocles gutturalis
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 36 - protein, 385 aa, Falco peregrinus
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 37 - protein, 386 aa, Phalacrocorax carbo
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 38 - protein, 386 aa, Merops nubicus
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 39 - protein, 386 aa, Tauraco erythrolophus
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 40 - protein, 386 aa, Cuculus canorus
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 41 - protein, 386 aa, Antrostomus carolinensis
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 42 - protein, 386 aa, Opisthocomus hoazin
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 43 - protein, 386 aa, Lepidothrix coronata
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 44 - protein, 386 aa, Struthio camelus
  source 1..386: note = Struthio camelus australis
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 45 - protein, 386 aa, Acanthisitta chloris
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 46 - protein, 381 aa, Tyto alba
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 47 - protein, 391 aa, Phalacrocorax carbo
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 48 - protein, 383 aa, Pipra filicauda
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 49 - protein, 386 aa, Dromaius novaehollandiae
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 50 - protein, 392 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 51 - protein, 386 aa, Corapipo altera
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 52 - protein, 395 aa, Amazona aestiva
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 53 - protein, 386 aa, Melopsittacus undulatus
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 54 - protein, 386 aa, Neopelma chrysocephalum
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 55 - protein, 385 aa, Buceros rhinoceros
  source 1..385: note = Buceros rhinoceros silvestris
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 56 - protein, 386 aa, Cariama cristata
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 57 - protein, 383 aa, Manacus vitellinus
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 58 - protein, 386 aa, Empidonax traillii
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 59 - protein, 386 aa, Leptosomus discolor
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 60 - protein, 2174 aa, Colinus virginianus
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 61 - protein, 386 aa, Apteryx australis
  source 1..386: note = Apteryx australis mantelli
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 62 - protein, 2104 aa, Callipepla squamata
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 63 - protein, 386 aa, Mesitornis unicolor
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 64 - protein, 420 aa, Anas platyrhynchos
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 65 - protein, 386 aa, Chaetura pelagica
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 66 - protein, 386 aa, Apaloderma vittatum
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 67 - protein, 386 aa, Corvus cornix
  source 1..386: note = Corvus cornix cornix
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 68 - protein, 386 aa, Calypte anna
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 69 - protein, 386 aa, Corvus brachyrhynchos
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 70 - protein, 399 aa, Hirundo rustica
  source 1..399: note = Hirundo rustica rustica
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 71 - protein, 389 aa, synthetic construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 72 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 73 - protein, 389 aa, synthetic construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 74 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP